(19) United States
(12) Patent Application Publication
(10) Pub. No.: US 2025/0327812 A1
(43) Pub. Date: Oct. 23, 2025

(54) Methods for Isolation, Enrichment, and Fractionation of Extracellular Vesicles

(71) Applicant: Northeastern University, Boston, MA (US)

(72) Inventors: Alexander R. IVANOV, Newton, MA (US); Xianyi SU, Boston, MA (US); Alan ZIMMERMAN, Boston, MA (US)

(21) Appl. No.: 19/184,869

(22) Filed: Apr. 21, 2025

Related U.S. Application Data
(60) Provisional application No. 63/636,707, filed on Apr. 19, 2024.

(51) Int. Cl. G01N 33/68; G01N 1/34; G01N 33/574

(52) U.S. Cl. CPC G01N 33/6842; G01N 1/34; G01N 33/57488; G01N 33/57496

(57) ABSTRACT

Methods are provided for isolation, purification, enrichment, and/or fractionation of extracellular vesicles (EVs) using a multi-dimensional chromatography approach. The use of two or more different types of chromatography, either sequentially or simultaneously, yields superior removal of extraneous proteins and fractionation into sub-populations of EVs. The two or more different types of chromatography can be based on different modes of separation, such as separation by charge and by size, or can be separation by size using different size ranges. Analysis of the obtained EVs, such as by proteomics through mass spectrometry, can be used to diagnose a disease or medical condition based on EV-associated biomarkers.

### STATEMENT REGARDING FEDERALLY SPONSORED RESEARCH OR DEVELOPMENT

This invention was made with government support under Grant Numbers 1R01CA218500-01A1 and 1R35GM136421-01 awarded by the National Institutes of Health. The Government has certain rights in the invention.

### BACKGROUND

Extracellular vesicles (EVs) are nanometer-scale, phospholipid bilayer membrane-enclosed globular entities actively secreted into the extracellular milieu by various cell types^{1, 2}. Non-vesicle extracellular particles (NVEPs) are similar to EVs in their low-nm size ranges and representation of molecular types, while they lack the lipid bilayer membrane and the vesicular morphology². EVs and NVEPs function as cellular messengers by transporting a diverse assortment of bioactive cargo, including proteins, lipids, glycans, and nucleic acids¹. Given their significant roles in intercellular communication and their involvement in physiological and pathological processes³, EVs have gained considerable attention as promising candidates for molecular characterization. Liquid chromatography-tandem mass spectrometry (LC-MS/MS) has been used for analyzing the complex proteome composition of EVs; however, it faces significant challenges in sensitivity, specificity, quantitative accuracy, and dynamic range, particularly when dealing with the diverse and often minute biomolecular content of these vesicles originated from complex source matrices⁴.

EVs can have different origins, and circulating EVs among them, specifically those derived from plasma, present a unique opportunity for minimally invasive biomarker discovery⁵. These vesicles act as surrogates of their parent cells, being easily obtained from plasma and other biofluids. This is advantageous compared to collecting tissue samples that require more invasive procedures. Proteomic profiling of plasma-derived EVs offers a more consistent and effective approach for biomarker identification over conventional plasma proteomics, which is often compromised by a high dynamic range and high-abundance free plasma proteins⁶. Notwithstanding these advantages, the isolation and characterization of circulating EVs are facing significant technical and methodological challenges. Widely used isolation methods, such as ultracentrifugation and size exclusion chromatography (SEC), are inefficient in separating high-abundance free plasma proteins/protein complexes from EVs, which affects the accurate identification and quantification of EV-specific biomolecules⁷. Furthermore, these methods lack the ability to fractionate EVs into subpopulations. Such fractionation is vital for a deeper understanding of EVs, as these subpopulations can exhibit unique characteristics that are indicative of their potential role in physiological or pathological processes.

### SUMMARY

The present technology provides methods for isolation, purification, enrichment, and/or fractionation of extracellular vesicles (EVs) using a multi-dimensional chromatography approach. As used herein, “multi-dimensional chromatography” refers to methods that include the use of any two or more different types of chromatography, either sequentially or simultaneously, in order to isolate, purify, enrich, or fractionate a population of EVs. The two or more different types of chromatography can be based on different modes of separation, such as separation by charge in one mode and separation by size in another mode, or can be based on using the same mode of separation but using two or more different chromatography media, such as differing in range of size fractionation or another parameter utilizing the same mechanism of separation but differing in, for example, extent of separation, resolution range, materials used, method of elution, or type of sample acted upon. The use of two or more different chromatography media for size exclusion chromatography, each having a different useful range of size fractionation, is an example of multi-dimensional chromatography as used herein.

EVs are known to be heterogeneous in size and molecular composition, and the present technology leverages such differences, such as differences in inherent surface charge, size, and composition of different EV subpopulations, which allows them to be differentiated from one another and from other components of their source. The present methods can reduce the sample complexity and increase the purity of an EV population or sub-population. The resulting isolated, enriched, or fractionated EV populations or subpopulations can be analyzed and evaluated using a variety of techniques, including proteomic analysis, transmission electron microscopy or other forms of imaging in the submicron size range, nanoparticle tracking, and western blotting. EVs can be obtained from donors having various diseases or medical conditions and compared to EVs from healthy donors in order to perform diagnosis, prognosis, or to identify new biomarkers. The method was tested for its applicability to real-world specimens using a set of clinical prostate cancer samples and matched controls. The technology improves the isolation and fractionation of EV subpopulations and enhances EV-based diagnostics, biomarker discovery, and EV related research.

As used herein, the term “extracellular vesicles” or “EVs” includes both membranous vesicles and non-membranous particles.

One aspect of the technology is a method of isolation of EVs, including (a) providing a liquid sample comprising EVs, and (b) subjecting the sample to multi-dimensional chromatography, thereby providing isolated EVs. The liquid sample can be any bodily fluid obtained from a human or mammalian subject, or a fraction or preparation derived therefrom, such as using one or more pre-purification steps involving filtration or centrifugation, or it can be a sample of a cell culture medium or supernatant from centrifugation of cells, homogenized cells, or from an environmental sample. The sample can also be a homogenate of any tissue, organ, or collection of cells from such a subject, or from cell culture. Although the sample is preferably cell-free, it may contain cell fragments, cell organelles, or other components of an extracellular environment, such as blood or serum proteins. While the sample is a liquid sample, it can be a suspension containing molecular and supermolecular structures (e.g, nanoparticles, microparticles, or lipid bilayer membrane-enclosed vesicles) in the nanometer range (1-999 nm in size) or in the micrometer range (1-999 microns in size). The multi-dimensional chromatography is performed, as described throughout the present disclosure, either in two or more stages or in a single stage. If performed in a column format, the multi-dimensional chromatography can be performed sequentially using two or more different columns, or using two or more different chromatography media packed into a single column, either as a mixed bed or as two or more separate beds. Two or more dimensions or modes of chromatography can even be embodied in a single type of chromatography medium that separates sample components simultaneously based on two or more features, such as surface charge and particle size. Sample components separated by any dimension or mode of chromatography can be collected as a series of fractions or as a single fraction. Elution of sample components from a chromatography medium can be stepwise or continuous. Collected sample fractions can be analyzed by any criteria and may be selected and/or pooled for use in a subsequent type of chromatography.

Another aspect of the present technology is a method of diagnosis or prognosis of a medical condition or disease. The method includes the following steps: (a) providing a sample from a subject suspected of having a medical condition or disease; (b) optionally processing the sample to provide a liquid sample suitable for use in a multi-dimensional chromatography method; (c) performing the multi-dimensional chromatography method; and (d) providing a diagnosis or prognosis of the medical condition or disease based on determination of one or more EV-related components associated with or diagnostic for the medical condition or disease.

Yet another aspect of the present technology is a method of identifying an EV-associated biomarker for a biological state, medical condition, or disease. The method includes the following steps: (a) performing the above-described method of using multi-dimensional chromatography to isolate, enrich, or fractionate a liquid sample obtained or derived from a subject having said biological state, medical condition, or disease, thereby obtaining isolated, enriched, or fractionated EVs, and performing analysis, such as by proteomics or another method, to characterize candidate biomarkers of the EVs; (b) comparing the results obtained in (a) with results of molecular and/or morphological analysis representing the lack of said biological state, medical condition, or disease; and (c) identifying one or more EV-associated biomarkers for the biological state, medical condition, or disease.

Still another aspect of the present technology is a kit containing a chromatography device, instructions for carrying out any of the methods disclosed herein for isolating, enriching, or fractionating a sample containing EVs, and optionally one or more reagents for use in the process of isolating, enriching, or fractionating the EVs, or for detection of an EV-associated biomarker. The kit may also contain instructions for performing a method of the present technology, and/or for the diagnosis of a disease or medical condition detectable by analysis of EVs or an EV sub-population.

Another aspect of the present technology is a system for isolation, purification, enrichment, fractionation, and/or analysis of EVs. The system can include a chromatography device, such as a chromatography medium or column, a liquid chromatography system capable of use with the chromatography device, such as a system for performing liquid chromatography and mass spectrometry, and a processor and memory comprising instructions for carrying out any of the methods of the present technology.

The present technology can be further summarized with the following list of features.
1. A method of isolation of extracellular vesicles (EVs), the method comprising:
   (a) providing a liquid sample comprising EVs;
   (b) subjecting the sample to multi-dimensional chromatography, thereby providing isolated EVs.
2. The method of feature 1, wherein the multi-dimensional chromatography comprises use of charge-based chromatography and at least one other chromatography method.
3. The method of feature 2, wherein the charge-based chromatography comprises anion exchange chromatography.
4. The method of feature 3, wherein the method comprises binding components of said liquid sample, or a sample derived therefrom, to an anion exchange chromatography medium followed by elution of EVs from the anion exchange chromatography medium using a gradient of pH or ionic strength.
5. The method of feature 4, wherein the gradient of pH or ionic strength is stepwise or linear.
6. The method of feature 5, wherein elution of EVs or EV sub-populations comprises stepwise elution of successive fractions using a series of buffered solutions of decreasing pH.
7. The method of any of the preceding features, wherein the multi-dimensional chromatography comprises size exclusion chromatography.
8. The method of feature 7, wherein the size exclusion chromatography comprises use of two or more different size exclusion chromatography media, each having a different pore size range and size fractionation range.
9. The method of feature 8, wherein an EV sub-population eluted from a first size exclusion chromatography medium is further fractionated using a second size exclusion chromatography medium having a smaller pore size range and size fractionation range than the first size exclusion chromatography medium.
10. The method of feature 9, wherein an EV sub-population eluted from the second size exclusion chromatography medium is further fractionated using a third size exclusion chromatography medium having a smaller pore size range and size fractionation range than the second size exclusion chromatography medium.
11. The method of any of the preceding features, wherein the multi-dimensional chromatography comprises ion exchange chromatography and size exclusion chromatography.
12. The method of feature 11, wherein the ion exchange chromatography is anion exchange chromatography.
13. The method of any of the preceding features, wherein the multi-dimensional chromatography comprises performing two or more different types of chromatography using a single chromatography column or multiple chromatography columns.
14. The method of any of the preceding features, wherein the multi-dimensional chromatography comprises subjecting the liquid sample, or a sample derived therefrom to chromatography using a chromatography medium comprising porous beads having internal charged groups, wherein said chromatography medium is capable of performing simultaneous size-based fractionation and anion exchange.
15. The method of any of the preceding features, wherein the multi-dimensional chromatography comprises affinity chromatography.
16. The method of feature 15, wherein the affinity chromatography comprises immunoaffinity chromatography, and wherein an EV-associated biomarker is used as affinity ligand.
17. The method of any of the preceding features, further comprising subjecting the liquid sample to centrifugation and/or filtration, whereby cells, subcellular components, and/or lipids are removed from the liquid sample prior to performing said multi-dimensional chromatography.
18. The method of any of the preceding features, wherein the liquid sample is, or is derived from, a liquid biopsy specimen, a cell culture medium, a cell lysate, a biological sample, a clinical sample, or an environmental sample.
19. The method of any of the preceding features, wherein the isolated EVs are enriched in EVs compared to the liquid sample.
20. The method of any of the preceding features, wherein the isolated EVs are fractionated into two or more fractions enriched with different EV subpopulations.
21. The method of feature 20, wherein the two or more EV subpopulations differ from one another by EV surface charge distribution or mean EV surface charge.
22 The method of any of features 20-21, wherein the two or more EV subpopulations differ from one another by EV size distribution or mean EV size.
23. The method of any of any of the preceding features, wherein the two or more EV subpopulations differ from one another by EV protein, lipid, glycan, or nucleic acid composition.
24. The method of any of any of the preceding features, further comprising subjecting the two or more EV subpopulations to a molecular analysis and/or morphological analysis.
25. The method of feature 24, wherein the molecular analysis and/or morphological analysis comprise one or more of proteomic analysis, lipidomic analysis, metabolomic analysis, glycomics analysis, transcriptomics analysis, targeted mass spectrometry, immunoaffinity methods, flow cytometry, biomarker analysis, imaging by electron microscopy, fluorescence microscopy, particle size analysis, or any combination thereof.
26. The method of feature 25, wherein the proteomic analysis comprises mass spectrometry.
27. The method of any of features 20-26, wherein the two or more EV subpopulations differ from one another in protein composition.
28. The method of feature 27, wherein the two or more EV subpopulations differ in presence, absence, or amount of one or more oncogenic proteins, tumor suppressor proteins, tetraspanins, lipoproteins, RNA-binding proteins, histones, mitochondrial proteins, plasma proteins, or any combination thereof.
29. The method of any of the preceding features, wherein at least one of said two or more EV subpopulations comprises one or more components associated with or diagnostic for a biological state, medical condition, or disease.
30. The method of feature 29, wherein said one or more components are diagnostic for a disease selected from the group consisting of cancers, neurodegenerative diseases, cardiovascular diseases, autoimmune diseases, infectious diseases, and aging related diseases.
31. The method of feature 30, wherein the disease is a cancer selected from the group consisting of melanoma, glioma, prostate cancer, breast cancer, cervical cancer, colorectal cancer, kidney cancer, lung cancer, lymphoma and pancreatic cancer.
32. The method of any of the preceding features, wherein the liquid sample comprises plasma, and the isolated EVs are at least partially separated from one or more plasma proteins and/or from one or more lipoprotein particle types.
33. A method of diagnosis or prognosis of a medical condition or disease, the method comprising:
   (a) providing a sample from a subject suspected of having the medical condition or disease;
   (b) optionally processing the sample to provide a liquid sample suitable for use as the liquid sample of any of the preceding features;
   (c) performing the method of any of the preceding features using the sample of (a) or the provided liquid sample of (b) as the liquid sample of said method; and
   (d) providing a diagnosis or prognosis of the medical condition or disease based on said determination of one or more components associated with or diagnostic for the medical condition or disease.
34. A method for identifying an EV-associated biomarker for a biological state, medical condition, or disease, the method comprising:
   (a) performing the method of any of features 24-32, wherein the liquid sample is obtained or derived from a subject having said biological state, medical condition, or disease, and whereby results of said proteomics analysis are obtained;
   (b) comparing the results obtained in (a) with results of molecular and/or morphological analysis representing the lack of said biological state, medical condition, or disease; and
   (c) identifying one or more EV-associated biomarkers for said biological state, medical condition, or disease.
35. The method of feature 34, wherein the molecular analysis and/or morphological analysis comprise one or more of proteomic analysis, lipidomic analysis, metabolomic analysis, glycomic analysis, targeted mass spectrometry, direct charge-based detection mass spectrometry, immunoaffinity methods, flow cytometry, imaging by electron microscopy or fluorescence microscopy, or any combination thereof.
36. A kit comprising a chromatography device, instructions for carrying out the method of any of the preceding features, and optionally a reagent such as an antibody for detection of an EV-associated biomarker.
37. A system for isolation, purification, and/or analysis of EVs, the system comprising a chromatography device, a liquid chromatography system capable of use with the chromatography device, and a processor and memory comprising instructions for carrying out the method of any of features 1-32.

As used herein, “consisting essentially of” allows the inclusion of materials or steps that do not materially affect the basic and novel characteristics of the claim. Any recitation herein of the term “comprising”, particularly in a listing of components of a composition or elements of a device, constitutes inclusion of alternative embodiments in which “comprising” is replaced with “consisting essentially of” or “consisting of”.

While the present invention has been described in conjunction with certain preferred embodiments, one of ordinary skill, after reading the foregoing specification, will be able to effect various changes, substitutions of equivalents, and other alterations to the compositions and methods set forth herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows an illustration of workflow for isolation, purification, and fractionation of EVs. Stage 1 represents method development and optimization (following black arrows) and Stage 2 shows pilot testing of clinical samples (following red arrows). Both stages began with whole blood collection from donors, followed by cell- and platelet-free plasma preparation. An additional centrifugation step at 18,000×g for 10 min was employed to remove interfering lipids. After centrifugation, the lower part of the supernatant plasma was carefully aspirated and introduced into equilibrated Capto Core 700 resin (resin/plasma ratio was 3 mg/μL) within a spin column, followed by a 30-min incubation on a rotating mixer. The eluate, designated as the Starting (ST) sample, was then collected in a tube after centrifugation. Subsequently, the ST sample was added to equilibrated Q-Sepharose resin (resin/plasma ratio was 8 mg/μL) in a spin column, and a series of step-gradient pH buffer elutions generated six eluate fractions, designated as Flow-Through (FT), pH5, pH4, pH3, pH2, and Final Elution (FE) fractions. All seven samples from the method development stage underwent LC-MS/MS-based proteomic analysis, supplemented by orthogonal techniques, including TEM, western blotting, and NTA. Conversely, during pilot analyses of clinical samples, only the EV-enriched fractions (i.e., the pH3, pH2, and FE fractions) were subjected to proteomic analysis.

FIGS. 2A-2C show results of EV characterization by methods other than LC-MS/MS. FIG. 2A shows western blot analysis demonstrating the fractionation/depletion patterns of six critical proteins, including three prevalent plasma proteins (albumin, IgG, and apoB-100) and three EV-related proteins (CD9, integrin β1, and Rap-1b), across the ST and six discrete fractions. FIG. 2B shows results of TEM and immunogold labeling. TEM images of EV-enriched samples (ST, pH2, and FE) highlight the presence of characteristic EV particles, predominantly displaying a “saucer” or “cap” shape with a dented center. Immunogold labeling targeting the CD9 protein (lower row of images) further confirms the efficiency of EV isolation and fractionation. FIG. 2C (NTA) provides a profile of the size distribution, particle concentration, and zeta potential of EVs for the ST, pH2, and FE samples.

FIGS. 3A-3D show multifaceted proteomic analysis. FIG. 3A shows intercorrelation analysis which highlights high reproducibility across sample datasets with Euclidean distance-based unsupervised hierarchical clustering, revealing detailed grouping patterns among EV-enriched fractions, e.g., FE, pH2, and starting sample, ST. FIG. 3B shows principal component analysis (PCA). The main figure visualizes distinct proteomic profiles of ST and all SAX fractions. The nested inset figure, excluding ST, emphasizes subtle proteomic profile differences between FE and pH2 and the method efficacy in fractionation. The ellipses represent 95% confidence levels. FIG. 3C shows a clustering heatmap, which demonstrates variations in protein abundance among samples, showcasing the fractionation method's ability to differentiate protein profiles and EV subpopulations, highlighted by three distinct clusters: the FE cluster (red arc-embraced sector), shared cluster (cyan arcs), and ST cluster (dark blue arcs). FIG. 3D shows the results of gene ontology (GO) enrichment analysis. The Krona pie chart illustrates the 10 most significant GO terms for the three clusters identified in FIG. 3C. The sector area of each GO term in the chart is proportional to its-log 10 (p-value). The FE cluster is characterized by GO terms predominantly associated with EVs (indicated in blue font) coming from cytosol and organelle origins. In contrast, the shared cluster encompasses terms mainly related to blood cell-originated (blood microparticle GO terms) and cell membrane-origin vesicles, along with GO terms associated with plasma proteins, highlighted in magenta font. Notably, the ST cluster features terms related to high-density lipoprotein (HDL) particles. This analysis demonstrates a higher rate of plasma protein depletion in the EV fractions compared to the ST sample. Furthermore, the distinct origins of EVs between the fractions suggest the effective fractionation into EV subpopulations.

FIGS. 4A-4B show detailed proteomic profiling and subcellular origin annotation of unique proteins from EV-enriched fractions. The Venn diagram in FIG. 4A illustrates the unique and commonly detected proteins between the examined fractions. For example, 43 and 7 unique proteins were identified in the FE and pH2 fractions, respectively, highlighting the distinct proteomic features of these EV-rich fractions. Thirty-nine proteins were commonly detected in both the FE and pH2 fractions. The circular heatmap in FIG. 4B displays the relative abundance levels of these pinpointed proteins. Proteins from cell membranes and cytosol, indicative of EV constituents, are shown in green and blue fonts, respectively. Eleven major plasma proteins (red font) are also mapped alongside, providing insights into their elution patterns.

FIGS. 5A-5D show comparative proteomic analysis of disease vs. control groups. FIG. 5a shows a PCA plot illustrating the variance in the full proteomic profiles between Disease and Control groups. The first two principal components, PC1 and PC2, represent 37.2% and 8.3% of the total variance. The size of the 95% confidence ellipse indicates variability within each group, with the disease cohort showing a broader range in proteomic expression. FIG. 5B shows a volcano plot displaying differentially abundant proteins (DAPs) in prostate cancer, highlighting 37 upregulated and 2 downregulated proteins with a fold change >2 or <0.5 and a p-value <0.05 criteria. FIG. 5C shows a hierarchical clustering heatmap. Row-wise analysis reveals distinct clusters based on protein abundance, including a separation of the two downregulated proteins from the rest of the upregulated protein clusters. The heatmap also displays subclusters of secreted, cytosolic, and membrane proteins, suggesting their varied roles in the disease and relations to EVs. Column-wise analysis illustrates distinct clusters among the Disease and Control samples, reflecting the complex interplay of factors influencing protein expression in these groups. FIG. 5D shows KEGG pathway annotations that reveal cancer-relevant pathways for 22 out of 39 DAPs.

FIGS. 6A-6B show hierarchical clustering and comparison of DAPs Identified in the three EV-rich fractions. FIG. 6A shows hierarchical clustering heatmaps of DAPs identified in the pH3, pH2, and FE fractions, respectively. Uniquely identified proteins are shown in bold font. Asterisks show the proteins that were represented by the corresponding transcriptomic profiles in the TCGA data available for PCa. FIG. 6B shows a Venn diagram of the upregulated proteins for the three EV-rich fractions.

FIGS. 7A-7B show representative examples of results acquired during method development focused on balancing sample purity and EV recovery rate. FIG. 7A shows results from Capto Core 700 pre-purification. Examples are shown of four Capto Core 700 resin to plasma ratios (2.5, 3.0, 3.5, and 4.0 mg/μL) that were evaluated in duplicate using SDS-PAGE silver staining (top panel) and anti-CD9 western blotting (bottom panel). The SDS-PAGE gel allowed identification of major plasma proteins, including albumin (˜67 kDa MW) and IgG (˜50 kDa heavy chain and ˜25 kDa light chain) based on their migration relative to the protein ladder. Concurrently, specific CD9 bands are identified on the western blot membrane. A ratio of 3.0 was determined to offer the best compromise between purity and EV recovery rate. FIG. 7B shows Q-Sepharose fractionation by anion exchange. Resin to plasma ratios of 8.0 and 5.0 were examined in the shown examples. The anti-CD9 western blot (bottom panel) demonstrated enhanced EV recovery, and the SDS-PAGE gel (top panel) showed a clearer fractionation pattern of plasma proteins, indicating that a ratio of 8.0 is appropriate for reaching the desired performance.

FIGS. 8A-8E show representative TEM Images for the FT (FIG. 8A), pH5 (FIG. 8B), pH4 (FIG. 8C), and pH3 (FIG. 8D) fractions and a Negative Control (FIG. 8E) of a PBS blank. No prominent vesicles or particles could be identified in these samples.

FIG. 9 shows results of GO enrichment analysis for the clusters shown in FIG. 3C (full version).

FIGS. 10A and 10B show proteomics data evaluation for disease and control samples. FIG. 10A shows a data missingness map for the acquired proteomic data, revealing an average missing rate of 18.3%. FIG. 10B is an assessment of data normality of individual group and homoscedasticity between them, using the Shapiro-Wilk and Levene's tests, respectively. An adjusted p-value <0.05 indicates deviations from normality or homoscedasticity.

FIG. 11 shows quantitative transcriptomic profiles for selected genes corresponding to the detected eight DAPs based on the TCGA Gene Expression Data in PRAD (prostate adenocarcinoma; TCGA-PRAD). Adapted from UALCAN⁷. Prior art.

FIG. 12A shows receiver operating characteristic (ROC) curves for eight selected DAPs between PCa and control groups. ITIH2 demonstrated excellent diagnostic performance based on the quantitative proteomics data (AUC=0.91). C4BPB and CST1 showed AUCs of 0.82 and 0.80, respectively. GGCT, C1QTNF3, C1R, and HPX exhibited AUCs ranging from 0.75 to 0.79. F12 indicated a lower discriminative ability with an AUC of 0.68. FIG. 12B shows the ROC curve for ITIH2 and C4BPB developed based on the quantitative proteomic profiles, which demonstrated high diagnostic accuracy and sensitivity with an AUC of 0.91, indicating the potential for distinguishing disease states with this combined signature set.

FIGS. 13A-13E show assessment of 2D-SEC for isolation of plasma-derived EVs utilizing Sephacryl resins. FIG. 13A shows a schematic representation of the general workflow for 2D-SEC isolation followed by intact EV and protein characterization. FIG. 13B shows scanning electron microscopy images of a single Sephacryl 500 bead, showing spherical shape with no visible damage or aberrations during manufacturing. Zooming into the surface of the bead, the pores become visible; pore size is approximately <100 nm. FIG. 13C shows scanning electron microscopy images of a single Sephacryl 1000 bead, also indicating spherical shape with no visible damage or aberrations during manufacturing. The surface morphology shows larger pores in its surface compared to Sephacryl 500. Zooming into the surface of the bead, the large pores become visible; pore size is approximately 500-1000 nm. FIG. 13D shows particle concentration of each fraction collected by 1D-SEC measured by NTA. FIG. 13E shows the particle concentration of each fraction collected by 2D-SEC measured by NTA. A clear particle peak is observed ranging from fractions 10-22.

FIGS. 14A-14F show characterization of 1D- and 2D-SEC for isolation of plasma-derived EVs. FIG. 14A shows transmission electron microscopy images of intact EVs in each fraction, which qualitatively show that EVs are collected in each fraction. FIG. 14B shows the particle size distribution of the starting plasma material and in 1D-SEC and 2D fractions measured by NTA. The 2D-chromatographic particle peak is retained even after combining fractions together. FIG. 14C shows particle size of the starting plasma material, and in 1D-SEC and 2D fractions measured by NTA. Particle size increases in the 2D fractions, with size decreasing as particles elute from the column. FIG. 14D shows SDS-PAGE stained by Coomassie, indicating detection of many proteins within each fraction, with several bands exhibiting higher or lower intensities. FIG. 14E shows western blot analysis of each fraction. Three plasma proteins were blotted: apolipoprotein B100, albumin, and IgG. Two EV-related proteins were blotted: β-catenin and CD9. 50 nL plasma was used for both SDS-PAGE and western blot so as to not overload the gel.

FIGS. 15A-15G show proteomic profiling of plasma-derived EVs isolated from 1D- and 2D-SEC. FIG. 15A shows the results of unsupervised hierarchical clustering of 1D- and 2D-SEC, revealing distinct fractionation between the identified proteins within each fraction. FIG. 15B shows an extracted heat map of EV-related protein abundances, which illustrates the consistency of EVs and their fractionation. FIG. 15C shows an extracted heat map of apolipoproteins, indicating their highest abundances after 1D-SEC followed by depletion in 2D-SEC. FIG. 15D shows an extracted heat map of top plasma proteins, revealing similar abundances across fractions. FIG. 15E show a Venn diagram of all proteins from the heat map in FIG. 15A, with the 2D fractions pooled together as 2D-SEC. There are 112 unique proteins identified in 2D-SEC fractions that were not identified in the 1D-SEC sample, indicating deeper proteomic profiling. FIG. 15F shows that GO Term analysis of the top 5 cellular locations of the 112 unique proteins found in 2D-SEC are EV-related locations, such as cytoplasm, exosomes, nucleus, lysosomes, and plasma membrane. FIG. 15G shows GO Term analysis of the top 5 biological functions of the 112 unique proteins found in 2D-SEC, as expected in EV-related proteins, namely from protein metabolism, signal transduction, cell communication, energy pathways, and cell growth/maintenance.

FIGS. 16A-16D show assessment of plasma-derived EVs from healthy control and prostate cancer donors prior to 2D separation. FIG. 16A presents transmission electron microscopy (TEM) images of plasma-derived EVs from healthy control donors. FIG. 16B shows plasma-derived EVs from prostate cancer donors. The isolated EVs exhibit cupping, the classic characteristic of an indentation at the center of the EVs. They range in size ˜30-150 nm in diameter. All images are on the same scale. FIG. 16C shows particle concentrations of healthy control and PCa donor EVs determined by NTA. Average particle concentrations are similar. FIG. 16D shows particle size of healthy control and PCa donor EVs by NTA. Particle sizes are similar and reproducible.

FIG. 17A shows representative TEM images of negative controls, dPBS, void volume after control 1D-SEC, and void volume after 2D-SEC. All images are on the same scale. FIG. 17B shows particle concentration of 1D- and 2D-SEC void volumes from control plasma. FIG. 17C shows a bar chart of particle concentration from FIG. 13D.

FIG. 19A is a heat map showing abundances of apolipoproteins in 1D-SEC, 2D-SEC, and 2D-SEC with all fractions recombined (remix). All apolipoprotein abundances in the remix sample are less than or equal to abundances within 2D-SEC fractions, indicating depletion is occurring during the 2D-SEC isolation. FIG. 19B shows log 2 abundance of apolipoprotein AI and apolipoprotein B100 between 1D-SEC, 2D-SEC, and Remix. FIG. 19C shows raw abundance of apolipoprotein AI between 1D-SEC, 2D-SEC, and Remix. FIG. 19D Raw abundance of apolipoprotein B100 between 1D-SEC, 2D-SEC, and Remix.

FIG. 20A shows representative TEM images of void volumes from healthy control and PCa donors. FIG. 20B shows volcano plots from FIG. 18D, labels indicate the top 10 DEPs for each fraction as well as other proteins of interest.

FIG. 21 shows the results of GO term analysis.

FIG. 22 shows Kaplin-Meyer survival curves for YWHAQ, FEN1, and SLC25A5. 14-3-3 theta (YWHAQ, regulates kinase activity of PDPK1), flap endonuclease 1 (FEN1, interacts with DNA polymerase overexpression leads to cell proliferation), and ADP/ATP translocase 2 (SLC25A5, mediates import of ADP into the mitochondrial matrix for ATP synthesis and export of ATP into cell and acts as master regulator of mitochondrial energy output).

FIGS. 23A-B show comparative assessment of the depth of proteomic profiling of plasma-derived EVs. FIG. 23A shows log 2 N observations of the Human Plasma Peptide Atlas, Build 2023. FIG. 23B shows log 2 Sum Abundance of PCa donors. Protein index numbers of EV-related proteins decrease, indicating enrichment of EVs in PCa donors from the Human Plasma Peptide Atlas. 670 protein groups were quantified in PCa donors.

### DETAILED DESCRIPTION

The present technology provides a novel EV isolation approach which exploits the unique electrostatic properties of EVs. The charge-based fractionation method reduces contamination with high-abundance plasma proteins. More importantly, this approach facilitates the fractionation of plasma-derived EVs into distinct charge-based subpopulations, an approach not yet explored by existing methodologies. The approach is grounded in two inherent characteristics of EVs. First, the phosphate head groups of the phospholipids in the outer layer of the EV membrane possess negative charges that separate them from less charged plasma species, reducing the dynamic range and content complexity. Second, varying physiological or pathological conditions can make differences in surface glycocalyx (glycoproteins^{6, 9}, proteoglycans, glycolipids, etc.) and protein phosphorylation states, contributing to disparate charge densities and hence driving the fractionation of EV subpopulations. This differentiation in EV subpopulations originated at distinct biological and disease states would enhance the specificity and applicability of EV-based diagnostics.

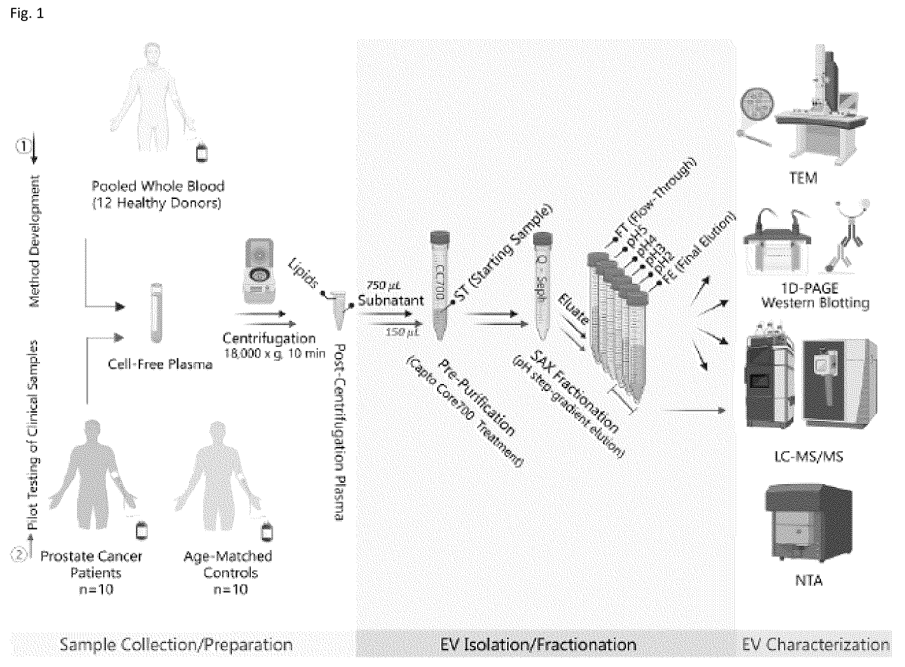

The inventors employed a two-tiered approach to test this hypothesis. Initially, they established the charge-based isolation method for EVs using plasma samples from self-declared healthy donors. Subsequently, they assessed its effectiveness using plasma samples collected from prostate cancer (PCa) patients and age-matched healthy controls (FIG. 1). The selection of PCa as an exemplary focus for this study is guided by several considerations. First, PCa is a prevalent cancer type in men. According to the American Cancer Society, there will be 299,010 new PCa cases and 35,250 deaths in the US in 2024¹⁰. Additionally, PCa is known to induce specific alterations in circulating EV profiles¹¹, making it an appropriate case for assessing the efficiency of our charge-based isolation method.

In the method development, optimization studies, and subpopulation enrichment validation, orthogonal techniques were implemented, including transmission electron microscopy (TEM), nanoparticle tracking analysis (NTA), and western blotting, in addition to LC-MS/MS-based proteomic analysis for a comprehensive EV characterization and method efficiency evaluation. Next, with an established method at the pilot testing stage for clinical samples, the focus was narrowed to LC-MS/MS proteomics to explore the potential applicability of the method in clinical diagnostics. The inventors were able to identify 39 differentially abundant proteins (DAPs) between the PCa and control groups, eight among which can be further corroborated by the findings generated using transcriptomics techniques and reported in The Cancer Genome Atlas (TCGA) and research publications.

The present technology is focused on the development of a straightforward, easy-to-use, and robust method for EV isolation and fractionation based on change. The approach provides a means for uncovering and characterizing a novel charge profile dimension of circulating EVs, which could be instrumental in refining diagnostic and therapeutic strategies for PCa and potentially a range of other cancers and pathological conditions.

For each experiment throughout this study, the volume of the samples was calculated based on the volume of plasma input and the dilution factors. For ease of reference, gene names used herein to represent the corresponding proteins are summarized in Table 1.

SUPPLEMENTARY TABLE 1
Gene Names of Proteins

| Gene | Protein |
|---|---|
| A2M | Alpha-2-Macroglobulin |
| ACAP1 | [symbol] with [symbol] and PH domains 1 |
| ADORF4 | Adhes [symbol] protein-coupled receptor F4 |
| ALB | Albumin |
| ALDOA | [symbol] A |
| A [symbol] 1 | [symbol] |
| A [symbol] | [symbol] |
| A [symbol] 2 | [symbol] |
| ATP [symbol] F1A | ATP synthase F1 subunit alpha |
| [symbol] 1 | Brain [symbol] membrane attached [symbol] protein 1 |
| C [symbol] QTNF3 | C [symbol] q and TNF related 3 |
| C3 | Complement C3 |
| C48 | Complement C48 |
| C48PB | C4b-binding protein beta chain |
| C5 | Complement C5 |
| [symbol] | [symbol] -1 |
| [symbol] | [symbol] -3 |
| C [symbol] SN | C [symbol] |
| CPAA | C [symbol] AA |
| CS [symbol] | [symbol] |
| CSTA | [symbol] |
| [symbol] | [symbol] 1 |
| F10 | Coagulation factor X |
| F12 | Coagulation factor XII |
| GAPDH | [symbol] |
| GGCT | Gamma- [symbol] |
| GP1BA | Glycoprotein [symbol] alpha chain |
| H2BC5 | Histone cluster 2 H2B family member c |
| [symbol] | [symbol] |
| HSPA5 | Heat shock 20 kDa protein 6 |
| IGHG | Immunoglobulin heavy constant gamma |
| IGHV6-1 | Immunoglobulin heavy variable 6-1 |
| IGKV [symbol] D-11 | Immunoglobulin kappa variable [symbol] D-11 |
| IGL1 | Immunoglobulin [symbol] -1 light chain |
| IGLC3 | Immunoglobulin [symbol] constant 3 |
| ITGA2B | Integrin alpha-IIb |
| [symbol] | Inter-alpha-trypsin inhibitor heavy chain [symbol] |
| MASP1 | [symbol] -binding lectin serine protease 1 |
| MPO | [symbol] |
| MUC2 | Mucin 2 |
| MUC58 | Mucin 58 |
| MUC6 | Mucin 6 |
| PF4V | Platelet Factor 4 variant |
| [symbol] | [symbol] |
| [symbol] | [symbol] 4 |
| [symbol] | [symbol] 3 |
| [symbol] | [symbol] -related protein [symbol] |
| [symbol] | [symbol] -related protein [symbol] |
| [symbol] | [symbol] -related protein [symbol] |
| [symbol] | [symbol] -related protein [symbol] |
| SERPINC2 | Serpin Family C member 2 |
| SERPINF2 | Serpin Family F member 2 |
| SERPING1 | Serpin Family G member 1 |
| SNAP23 | [symbol] 23 |
| [symbol] | [symbol] |
| TMSB10 | [symbol] beta-10 |
| TMSB4 [symbol] | [symbol] beta-4, [symbol] -linked |
| TNC | [symbol] |
| [symbol] SP | [symbol] -stimulated phosphoprotein |
| [symbol] | [symbol] |
| ZYX | [symbol] |

Based on www.uniprot.org [symbol]
indicates data missing or illegible when filed

### References—Set 1

1. Théry, C. et al. Minimal information for studies of extracellular vesicles 2018 (MISEV2018): a position statement of the International Society for Extracellular Vesicles and update of the MISEV2014 guidelines. Journal of Extracellular Vesicles 7, 1535750 (2018).
2. Welsh, J. A. et al. Minimal information for studies of extracellular vesicles (MISEV2023): 3 From basic to advanced approaches. Journal of Extracellular Vesicles 13, e12404 (2024).
3. Van Niel, G., D'Angelo, G. & Raposo, G. Shedding light on the cell biology of extracellular vesicles. Nature Reviews Molecular Cell Biology 19, 213-228 (2018).
4. Gandham, S. et al. Technologies and Standardization in Research on Extracellular Vesicles. Trends in Biotechnology 38, 1066-1098 (2020).
5. Szatanek, R., Baran, J., Siedlar, M. & Baj-Krzyworzeka, M. Isolation of extracellular vesicles: Determining the correct approach (Review). International Journal of Molecular Medicine 36, 11-17 (2015).
6. Kaur, G. et al. Extending the Depth of Human Plasma Proteome Coverage Using Simple Fractionation Techniques. Journal of Proteome Research 20, 1261-1279 (2021).
7. Taylor, D. D. & Shah, S. Methods of isolating extracellular vesicles impact down-stream analyses of their cargoes. Methods 87, 3-10 (2015).
8. Marie, A.-L., Ray, S. & Ivanov, A. R. Highly-sensitive label-free deep profiling of N-glycans released from biomedically-relevant samples. Nature Communications 14, 1618 (2023).
9. Marie, A.-L. et al. High-Sensitivity Glycan Profiling of Blood-Derived Immunoglobulin G, Plasma, and Extracellular Vesicle Isolates with Capillary Zone Electrophoresis-Mass Spectrometry. Analytical Chemistry 93, 1991-2002 (2021).
10. Siegel, R. L., Giaquinto, A. N. & Jemal, A. Cancer statistics, 2024. CA: A Cancer Journal for Clinicians 74, 12-49 (2024).
11. Vlaeminck-Guillem, V. Extracellular Vesicles in Prostate Cancer Carcinogenesis, Diagnosis, and Management. Frontiers in Oncology 8 (2018).
12. Perez-Riverol, Y. et al. The PRIDE database resources in 2022: a hub for mass spectrometry-based proteomics evidences. Nucleic Acids Research 50, D543-D552 (2022).
13. Kratz, F. & Elsadek, B. Clinical impact of serum proteins on drug delivery. Journal of Controlled Release 161, 429-445 (2012).
14. Phan, H. T. M., Bartelt-Hunt, S., Rodenhausen, K. B., Schubert, M. & Bartz, J. C. Investigation of Bovine Serum Albumin (BSA) Attachment onto Self-Assembled Monolayers (SAMs) Using Combinatorial Quartz Crystal Microbalance with Dissipation (QCM-D) and Spectroscopic Ellipsometry (SE). PLOS ONE 10, e0141282 (2015).
15. Dolan, J. Back to Basics: The Role of pH in Retention and Selectivity. 30, 30-33 (2017).
16. Buzás, E. I., Tóth, E. A., Sódar, B. W. & Szabó-Taylor, K. É. Molecular interactions at the surface of extracellular vesicles. Seminars in Immunopathology 40, 453-464 (2018).
17. Baranyai, T. et al. Isolation of Exosomes from Blood Plasma: Qualitative and Quantitative Comparison of Ultracentrifugation and Size Exclusion Chromatography Methods. PLOS ONE 10, e0145686 (2015).
18. Chiodi, F., Siden, A. & Osby, E. Isoelectric focusing of monoclonal immunoglobulin G, A and M followed by detection with the avidin-biotin system. ELECTROPHORESIS 6, 124-128 (1985).
19. Cobb, B. A. The history of IgG glycosylation and where the inventors are now. Glycobiology 30, 202-213 (2019).
20. Brennan, K. et al. A comparison of methods for the isolation and separation of extracellular vesicles from protein and lipid particles in human serum. Scientific Reports 10, 1039 (2020).
21. Andreu, Z. & Yáñez-Mo, M. Tetraspanins in Extracellular Vesicle Formation and Function. Frontiers in Immunology 5 (2014).
22. Toribio, V. & Yáñez-Mó, M. Tetraspanins interweave EV secretion, endosomal network dynamics and cellular metabolism. European Journal of Cell Biology 101, 151229 (2022).
23. Bromberger, T., Zhu, L., Klapproth, S., Qin, J. & Moser, M. Rap1 and membrane lipids cooperatively recruit talin to trigger integrin activation. Journal of Cell Science 132, jcs.235531 (2019).
24. Zhao, Z., Wijerathne, H., Godwin, A. K. & Soper, S. A. Isolation and analysis methods of extracellular vesicles (EVs). Extracellular Vesicles and Circulating Nucleic Acids 2, 80-103 (2021).
25. Rogers, M. A. et al. Annexin A1-dependent tethering promotes extracellular vesicle aggregation revealed with single-extracellular vesicle analysis. Science Advances 6, eabb1244 (2020).
26. O'Brien, K., Breyne, K., Ughetto, S., Laurent, L. C. & Breakefield, X. O. RNA delivery by extracellular vesicles in mammalian cells and its applications. Nature Reviews Molecular Cell Biology 21, 585-606 (2020).
27. Qian, L. et al. Reduced O-GlcNAcylation of SNAP-23 promotes cisplatin resistance by inducing exosome secretion in ovarian cancer. Cell Death Discovery 7, 112 (2021).
28. Li, J. et al. An ACAP1-containing clathrin coat complex for endocytic recycling. The Journal of Cell Biology 178, 453-464 (2007).
29. Piper, R. C. & Katzmann, D. J. Biogenesis and Function of Multivesicular Bodies. Annual Review of Cell and Developmental Biology 23, 519-547 (2007).
30. Gurung, S., Perocheau, D., Touramanidou, L. & Baruteau, J. The exosome journey: from biogenesis to uptake and intracellular signalling. Cell Communication and Signaling 19, 47 (2021).
31. Yap, B. W. & Sim, C. H. Comparisons of various types of normality tests. Journal of Statistical Computation and Simulation 81, 2141-2155 (2011).
32. Gastwirth, J. L., Gel, Y. R. & Miao, W. The Impact of Levene's Test of Equality of Variances on Statistical Theory and Practice. Statistical Science 24 (2009).
33. Smyth, G. K. Linear Models and Empirical Bayes Methods for Assessing Differential Expression in Microarray Experiments. Statistical Applications in Genetics and Molecular Biology 3, 1-25 (2004).
34. Ritchie, M. E. et al. limma powers differential expression analyses for RNA-sequencing and microarray studies. Nucleic Acids Research 43, e47-e47 (2015).
35. Kuang, Q. et al. The ALDOA Metabolism Pathway as a Potential Target for Regulation of Prostate Cancer Proliferation. OncoTargets and Therapy Volume 14, 3353-3366 (2021).
36. Ross, P. L. et al. Carboxypeptidase 4 gene variants and early-onset intermediate-to-high risk prostate cancer. BMC Cancer 9, 69 (2009).
37. Zhou, W., Grandis, J. R. & Wells, A. STAT3 is required but not sufficient for EGF receptor-mediated migration and invasion of human prostate carcinoma cell lines. British Journal of Cancer 95, 164-171 (2006).
38. Legrier, M.-E. et al. Mucinous differentiation features associated with hormonal escape in a human prostate cancer xenograft. British Journal of Cancer 90, 720-727 (2004).
39. Guo, C., Liu, S. & Sun, M.-Z. Novel insight into the role of GAPDH playing in tumor. Clinical and Translational Oncology 15, 167-172 (2013).
40. Lage-Vickers, S. et al. The expression of YWHAZ and NDRG1 predicts aggressive outcome in human prostate cancer. Communications Biology 4, 103 (2021).
41. Network, T. C. G. A. R. et al. The Cancer Genome Atlas Pan-Cancer analysis project. Nature Genetics 45, 1113-1120 (2013).
42. Chandrashekar, D. S. et al. UALCAN: A Portal for Facilitating Tumor Subgroup Gene Expression and Survival Analyses. Neoplasia 19, 649-658 (2017).
43. Chandrashekar, D. S. et al. UALCAN: An update to the integrated cancer data analysis platform. Neoplasia (New York, N. Y.) 25, 18-27 (2022).
44. Ricklin, D., Hajishengallis, G., Yang, K. & Lambris, J. D. Complement: a key system for immune surveillance and homeostasis. Nature Immunology 11, 785-797 (2010).
45. Nickel, K. F. et al. The polyphosphate-factor XII pathway drives coagulation in prostate cancer-associated thrombosis. Blood 126, 1379-1389 (2015).
46. Hisada, Y., Geddings, J. E., Ay, C. & Mackman, N. Venous thrombosis and cancer: from mouse models to clinical trials. Journal of Thrombosis and Haemostasis 13, 1372-1382 (2015).
47. Traverso, N. et al. Role of Glutathione in Cancer Progression and Chemoresistance. Oxidative Medicine and Cellular Longevity 2013, 1-10 (2013).
48. li, H. et al. A Novel Prodrug of a γ-Glutamylcyclotransferase Inhibitor Suppresses Cancer Cell Proliferation in vitro and Inhibits Tumor Growth in a Xenograft Mouse Model of Prostate Cancer. ChemMedChem 13, 155-163 (2018).
49. Hamm, A. et al. Frequent expression loss of Inter-alpha-trypsin inhibitor heavy chain (ITIH) genes in multiple human solid tumors: A systematic expression analysis. BMC Cancer 8, 25 (2008).
50. Bader, D. A. et al. Mitochondrial pyruvate import is a metabolic vulnerability in androgen receptor-driven prostate cancer. Nature Metabolism 1, 70-85 (2018).
51. Mostaghel, E. A. et al. Intraprostatic Androgens and Androgen-Regulated Gene Expression Persist after Testosterone Suppression: Therapeutic Implications for Castration-Resistant Prostate Cancer. Cancer Research 67, 5033-5041 (2007).
52. Ellem, S. J., Schmitt, J. F., Pedersen, J. S., Frydenberg, M. & Risbridger, G. P. Local Aromatase Expression in Human Prostate Is Altered in Malignancy. The Journal of Clinical Endocrinology & Metabolism 89, 2434-2441 (2004).

### EXAMPLES

### Example 1. Isolation, Enrichment, Fractionation, and Characterization of EVs Involving Anion Exchange

### Sample Collection and Preparation

For method development, plasma samples were obtained from 12 self-reported healthy male donors, aged 23 to 67, following IRB protocols IRB #2001P000591 (BIDMC) and IRB #17-12-14 (NU). Informed consent was secured from all donors. Ethylenediaminetetraacetic acid (EDTA) was used as the anticoagulant for blood collection, which was then pooled to form a representative healthy donor sample. Aliquots of 1 mL from this sample were cryopreserved at −80° C. Prior to EV isolation, these samples were thawed at 37° C. and centrifuged at 12,000×g for 10 min, targeting a reduction in lipid interference. The lower half of the plasma supernatant was carefully aspirated, avoiding the lipid-rich layer floating on the surface that formed post-centrifugation.

For clinical pilot testing, plasma samples from ten PCa patients (D_01 to D_10) and ten age-matched healthy controls (C_01 to C_10) were obtained from Lee BioSolutions (Maryland Heights, MO). An age stratification was applied with the selection of four ranges, namely ≤60 (groups 1, 2), 60-70 (groups 3, 4, 5), 70-80 (groups 6, 7, 8, 9), and >80 (group 10) years-old, respectively, to ensure varied age representation (Table 2). These samples underwent identical pre-processing steps as the healthy donor samples, ensuring analytical consistency across the study.

SUPPLEMENTARY TABLE 2
Clinical Sample Information

| | Pro [symbol] Cancer Patients | | | Controls | | |
|---|---|---|---|---|---|---|
| Age Group | Cat. [symbol] | Age | In-House Label | Cat. [symbol] | Age | In-House Label |
| 1 | 991-58-5-PC | 47 | D_01 | 991-58-P5 | 46 | C_01 |
| 2 | 991-58-5-PC | 58 | D_02 | 991-58-P5 | 58 | C_02 |
| 3 | 991-58-5-PC | 60 | D_03 | 991-58-P5 | 63 | C_03 |
| 4 | 991-58-5-PC | 65 | D_04 | 991-58-P5 | 66 | C_04 |
| 5 | 991-58-5-PC | 6 [symbol] | D_05 | 991-58-P5 | 71 | C_05 |
| 6 | 991-58-5-PC | 70 | D_06 | 991-58-P5 | 72 | C_06 |
| 7 | 991-58-5-PC | 74 | D_07 | 991-58-P5 | 75 | C_07 |
| 8 | 991-58-5-PC | 74 | D_08 | 991-58-P5 | 75 | C_08 |
| 9 | 991-58-5-PC | 7 [symbol] | D_09 | 991-58-P5 | 76 | C_09 |
| 10 | 991-58-5-PC | [symbol] | D_10 | 991-58-P5 | 86 | C_10 |

www.leeb [symbol] a.com [symbol]
indicates data missing or illegible when filed

SUPPLEMENTARY TABLE 3
Eluting Buffer Composition

| | 0.3% | 50 mM | 2.5% | 1.5% | |
|---|---|---|---|---|---|
| | Acetic | Ammonium | Acetic | Formic | |
| Eluting Buffer | Acid | Acetate | Acid | Acid | pH |
| pH 5 | [symbol] 0% | [symbol] 0% | — | — | 5.44 |
| pH 4 | [symbol] 0% | [symbol] 0% | — | — | 4.54 |
| pH 3 | [symbol] 5% | [symbol] % | — | — | 3.41 |
| pH 2 | — | — | 100% | — | 2.56 |
| [symbol] E [final Elution] | — | — | — | 100% | 2.02 |

[symbol]

indicates data missing or illegible when filed

### Materials

2-Iodoacetamide (IAA), acetic acid, ammonium bicarbonate, bovine serum albumin (BSA), and urea were purchased from Sigma-Aldrich. Capto Core 700 (CC700) resin, Dulbecco's Phosphate-Buffered Saline (dPBS), and Q-Sepharose resin, were procured from Cytiva. Acetonitrile (ACN), formic acid (FA), Tris(2-carboxyethyl) phosphine (TCEP), thiourea, skim milk powder, 20×PBS Tween™ 20 (PBST) buffer, SuperSignal West Femto maximum sensitivity substrate, Parafilm, and 5 mL centrifuge columns were obtained from Thermo Fisher Scientific. 4× Lithium dodecyl sulfate (LDS) sample buffer, 10× dithiothreitol (DTT), 4-12% Bis-Tris gel, and polyvinylidene difluoride (PVDF) membrane were purchased from Invitrogen. Amicon Ultra 10 kDa MWCO filter was purchased from MilliporeSigma. C18 membrane disk was acquired from CDS. A carbon film-supported copper gilder grid was obtained from Electron Microscopy Sciences. ReproSil-Pur 120 C18-AQ beads were purchased from Dr. Maisch. Trypsin/Lys-C mix was obtained from Promega. Anti-albumin (sc-271605), anti-apoB-100 (sc-13538), anti-IgG (sc-69786) antibodies, mouse anti-rabbit IgG-HRP secondary antibody (sc-2357), and HRP-conjugated mouse IgG light chain binding protein (sc-516102) were all purchased from Santa Cruz. Anti-CD9 (10626D), anti-integrin 131 antibodies (14-029982), and 10 nm colloidal gold-conjugated goat anti-mouse IgG secondary antibody (A-31561) were sourced from Invitrogen. Anti-Rap-1b (10840-1-AP) antibody was obtained from Proteintech.

### EV Characterization

### 1D-Polyacrylamide Gel Electrophoresis (1D-PAGE)

Starting sample ST and all fractions (15 iL plasma input equivalent) were concentrated using a FreeZone lyophilizer (Labconco, US), then mixed with 5 iL of 4×LDS sample buffer and 2 iL of 10×DTT for a total volume of 20 iL. This mixture was then heated at 70° C. for 10 min for lysis and denaturation. Electrophoresis was conducted on a 4-12% Bis-Tris gel at 200 V for 1 h.

### Western Blotting

Western blot analysis was performed on six proteins, categorized into three high-abundance plasma species (albumin, immunoglobulin G (IgG), and apolipoprotein B-100 (apoB-100)) and three EV-related proteins (CD9-antigen, integrin 131, and Ras-related protein Rap-1b). Each protein underwent individual blotting due to their significantly different abundances. The sample volumes, in terms of the plasma input, were as follows: 10 iL for albumin, 20 iL each for IgG and apoB-100, 50 iL each for CD9 and Rap-1b, and 100 iL for integrin 131. After performing the same lysis, denaturation, and electrophoresis protocols as in 1D-PAGE, proteins were transferred to a PVDF membrane using a Trans-Blot Turbo Transfer System (Bio-Rad, US). Distinctly, CD9 detection was performed under non-reducing conditions throughout the entire process to maintain epitope reactivity. After transfer, membranes were blocked for 1 h at room temperature with 5% skim milk in 1×PBST buffer, then incubated overnight at 4° C. with primary antibodies: anti-human albumin (1:500), IgG (1:1,000), apoB-100 (1:500), CD9 (1:750), integrin 131 (1:250), and Rap-1b (1:500). Subsequently, membranes were treated with HRP-conjugated mouse IgG light chain-binding protein (1:2,000), or mouse anti-rabbit IgG-HRP secondary antibody (1:1,000) for Rap-1b blotting, at room temperature for 1 h. Chemiluminescent signals were then activated using SuperSignal West Femto maximum sensitivity substrate, and the images were documented by a ChemiDoc MP Imaging System (Bio-Rad, US).

### Transmission Electron Microscopy Imaging

The TEM protocol for EV imaging began with a concentration step to increase the EV particle quantity. All samples, equivalent to a 50 iL plasma input volume, were concentrated to a final 20 μL volume using an Amicon Ultra 10 kDa MWCO filter in a microcentrifuge (Eppendorf, Germany) at 14,000×g for 30 min at 4° C. Subsequently, 5 μL of each concentrated sample was carefully placed onto a parafilm sheet. On the top of each sample-containing droplet, a glow-discharged 10 nm-thick carbon film-supported copper gilder grid was positioned for an incubation period of 15 min at room temperature. After incubation, grids were gently rinsed with water to remove any unattached or excess samples. For enhanced contrast, samples underwent negative staining using a 2% uranyl acetate solution for 2 min.

Additionally, to increase specificity, immunogold TEM was performed on EV-enriched samples (i.e., ST, pH2, and FE). This involved initially blocking the sample-loaded grids with 1% BSA for 10 min. The grids were then incubated for 30 min on a 5 μL drop of anti-human CD9 primary antibody diluted (1:15) in 1% BSA. This was followed by three 10-min washes with 1×dPBS. The grids were then incubated for 20 min with droplets of 10 nm colloidal gold-conjugated secondary antibody (1:30) in 1% BSA, and subsequently washed twice with 1×dPBS for 5 min each plus four times with water for 10 min each. Finally, the prepared samples were visualized using a JEM 1010 TEM microscope (JEOL Ltd., Japan) equipped with a 2k×2k pixels AMT XR-41B CCD camera system.

### Nanoparticle Tracking Analysis

NTA was conducted using a ZetaView instrument (Particle Metrix, Germany). Before the analysis, each sample was diluted 50 times to a final volume of 1 mL and then thoroughly vortexed to ensure homogeneity. This dilution step is crucial to avoid detector saturation with overabundant signals and achieve accurate measurements. During the analysis, the selected laser wavelength was 488 nm, and the filter was set to detect scattered light. The sample chamber of the instrument was maintained at room temperature. For measuring particle size distribution, the procedure included two cycles, each consisting of 11 different positions within the sample chamber. For zeta potential measurements, five cycles were conducted where two stationary layers were established at relative positions of 0.149 and 0.851 within the chamber to ensure accurate measurements.

### Nanoflow LC-MS/MS Proteomic Profiling

### Sample Preparation

The sample lysis/digestion preparation was performed with an optimized OmSET protocol¹ with minor changes to minimize sample loss. Specifically, 150 μL (plasma input volume equivalent) of each EV sample was introduced into a 200 μL pipet tip. This tip was pre-packed with four layers of a C18 membrane obtained using a blunt tip needle of 14-gauge. Lysis was performed for 20 min at room temperature using a mixture composed of 8 M urea, 2.5 M thiourea, and 6 mM TCEP in 25 mM ammonium bicarbonate at pH 8. This was followed by a simultaneous reduction and alkylation step with 25 mM TCEP and 10 mM IAA in 25 mM ammonium bicarbonate (pH 8) for 45 min in the dark at room temperature. Overnight proteolytic digestion was then executed at 45° C. using trypsin and Lys-C mix at a 1:10 enzyme-to-substrate ratio (for each enzyme). The digested peptides were subsequently eluted into glass LC inserts with three successive 10 μL aliquots of a solution of 65% ACN and 0.1% FA. These samples were then lyophilized to complete dryness and stored at −80° C. Prior to LC injection, they were reconstituted in a 5 μL solution of 1% ACN with 0.1% FA.

### Nanoflow LC Conditions

The nanoflow LC (nLC) separation of digested peptides employed an in-house packed C18 column. A fused silica capillary (75 μm ID×360 μm OD) was laser-pulled to produce an electrospray ionization (ESI) emitter tip. The pulled capillary was carefully packed with ReproSil-Pur 120 C18-AQ beads with a mean diameter of 1.9 μm and pore size of 120 Å. The length of the packed column was 15 cm.

For method development with healthy donor samples, the Ultimate 3000 nLC system (Thermo Fisher Scientific, US) was utilized. The analytical column was connected via a tee union to a nanoViper transfer line (20 μm ID×360 μm OD×1 m length) linked to the LC switching valve. The ESI voltage was applied at the tee union for ionization of analytes during elution. The pilot tests with clinical samples employed the Vanquish Neo UHPLC system (Thermo Fisher Scientific, US). In both setups, the column was housed in a pencil column heater (Phoenix S&T, US) and maintained at 60° C. to ensure consistent retention times and performance.

Chromatographic conditions involved a mobile phase A of 0.1% FA in water, while mobile phase B comprised 0.1% FA in ACN. With the Ultimate 3000 system, samples were loaded onto the column at 350 nL/min for 20 min, followed by an elution at 120 nL/min over a 120-min gradient from 1% B to 25% B. On the Vanquish Neo, the gradient was shortened to 45 min at 200 nL/min.

### Nanoflow LC-Tandem Mass Spectrometry (MS/MS)

For method development, each sample was subjected to triplicate (equivalent to 45 μL plasma input for one injection) nLC-MS/MS analysis employing an Exploris 480 Orbitrap mass spectrometer (Thermo Fisher Scientific, US). The ESI voltage was set at 1.8 kV using an EASY-Spray ionization source (Thermo Fisher Scientific, US), and the ion transfer tube temperature was held at 275° C. The system was set to operate in positive ESI and data-dependent acquisition (DDA) modes. For full MS¹ scans, the spectral range was 3751,600 m/z, and the resolution was 120,000 (at 200 m/z). Specific adjustments included a normalized automatic gain control (AGC) target at 300%, the maximum injection time on auto mode, microscans at a value of 1, and the RF lens intensity set to 50%. For MS₂ analysis, higher-energy collisional dissociation (HCD) was maintained at a normalized energy of 30%. Precursor ions were selected for fragmentation at a Top Speed mode, where 3 sec lasted between two master scans, targeting ions with charge states between 2 and 6 and exhibiting a minimum intensity of 5×E3. To mitigate redundancy in precursor ion selection, a dynamic exclusion of 45 sec and an isotope exclusion were used. MS² spectra were acquired at a resolution of 30,000 (at 200 m/z) with a defined isolation window of 2 m/z. Other settings included a standard automatic gain control (AGC) target, an auto-regulated maximum injection time, and microscans set to 1. The “define first mass” mode was selected and set to start with 110 m/z.

A similar analytical procedure was employed for clinical samples, except that samples were injected in duplicates (equivalent to 70 μL plasma input). ESI voltage was increased to 2 kV. The MS² resolution was heightened to 60,000 (at 200 m/z), and the isolation window was set at 3 m/z.

### Data Analysis

Acquired raw files were processed using the Proteome Discoverer software (v. 3.0, Thermo Fisher Scientific). These files were searched against the UniProtKB/SwissProt human database (Release 2020.01). For method development, the Sequest HT search engine was employed. This analysis operated with a mass tolerance of 5 ppm for precursor ions and 0.02 Da for fragment ions. For the clinical samples, an AI-based engine, CHIMERYS, was utilized for the spectral search using a fragment mass tolerance of 5 ppm. Both searches allowed for up to two missed cleavage sites per peptide, with the minimum peptide length set to seven amino acid residues. Carbamidomethylation of cysteine residues was selected as a static modification. Spectrum matching further benefited from INFERYS rescoring under automated mode. To ensure data reliability, a stringent false discovery rate (FDR) threshold of 1% was applied at both the peptide and protein levels. Quantitative analysis of the identified proteins was executed through a label-free quantification (LFQ) approach. This process leveraged unique peptides with their respective spectrum abundances. The mass spectrometry proteomics data have been deposited to the ProteomeXchange Consortium via the PRIDE2 partner repository with the dataset identifier PXD049702.

Further data analysis and visualization for intercorrelation analysis, principal component analysis (PCA), Venn diagrams, differential analysis (PCa vs. healthy control samples), and volcano plots, and Sankey diagrams were performed within R. Hierarchical clustering heatmaps were generated with the open-access TBtools-II (v1.120) software³ and R. KEGG (Kyoto Encyclopedia of Genes and Genomes) annotation was performed with DAVID Knowledgebase⁴. GO (Gene Ontology) enrichment was conducted in FunRich (v3.1.3)⁵.

### EV Isolation and Fractionation

Initial Purification with Capto Core 700

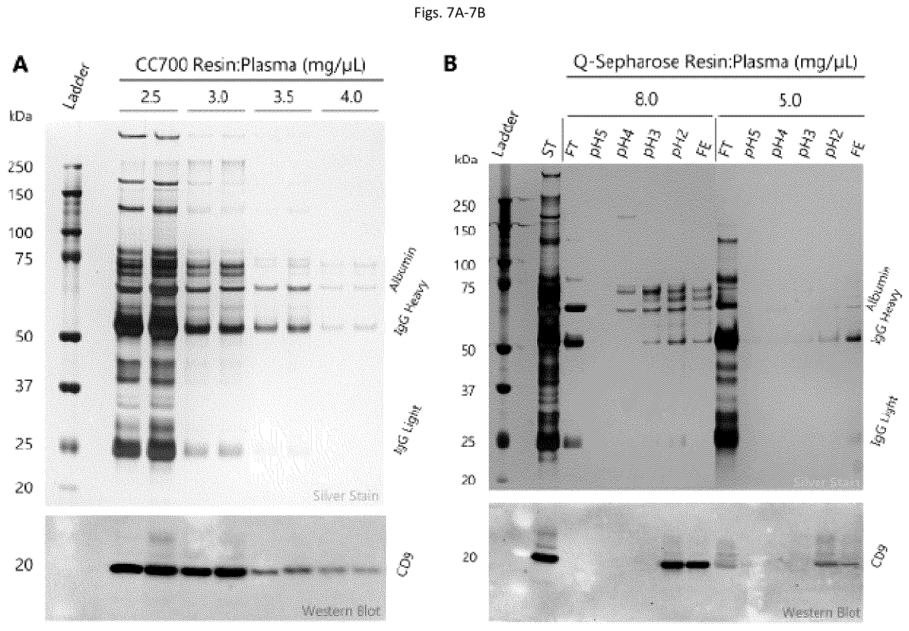

In the initial purification phase, a dual-functional chromatographic resin, Capto Core 700 (CC700), was used that possesses both size exclusion (molecular weight cut-off (MWCO)=700 kDa) and anion exchange capabilities. The resin was loaded into a 5 mL centrifuge column with a bottom frit. To ensure reproducibility, an accurate measurement of the resin dry mass, free from storage solution, was performed. This was achieved by weighing the empty column prior to its filling with resin. Subsequently, the column containing the resin was weighed after the removal of the storage solution by centrifugation at 4,500×g for 2 min. The dry mass of the resin was then determined by calculating the difference between the two weights. A 3.0 mg/μL resin-to-plasma ratio was applied in this step (FIG. 7A).

For method development, 750 μL of post-centrifugation plasma from pooled healthy donors was used. In clinical testing, only 150 μL of plasma was required from each patient or control sample. Prior to sample introduction, the resin was equilibrated with 0.1× Dulbecco's phosphate-buffered saline (dPBS) in a volume quadruple that of the plasma, ensuring a non-viscous mixture consistency and maximizing binding capacity. After a 30-min incubation at room temperature on a rotating mixer (Barnstead, US), the mixture was centrifuged at 4,500×g for 5 min. The resulting solution, designated as “ST” (starting sample), was meanwhile collected in a falcon tube (Plasma Dilution Factor=5).

### Fractionation Using Q-Sepharose

During the fractionation phase, Q-Sepharose strong anion exchange (SAX) resin was packed in a 5 mL centrifuge column with a bottom frit. For method development, half of the ST sample was processed, equating to 375 μL of plasma input, with the remaining half serving as a comparative sample. For clinical testing, the entire 150 μL of plasma input volume from the ST was used. An 8.0 mg/μL resin-to-plasma ratio was adopted (FIG. 7B).

The fractionation process commenced by introducing the ST sample into the Q-Sepharose resin, previously preconditioned with 0.1×dPBS at a volume five times the plasma input. After collecting the flow-through by centrifugation at 4,500×g for 3 min, a series of stepwise elutions was conducted with buffers at decreasing pH values (Table 3). Each elution involved a two-step process to ensure the efficient elution of bound analytes at the target pH and appropriate equilibration of the stationary phase at a specific pH, each step involving centrifugation at 4,500×g for 3 min, using buffer volumes five times the plasma input, resulting in a total of 10 times the plasma input volume for each fraction. The fractions were designated as “FT” (flow-through), “pH5”, “pH4”, “pH3”, “pH2”, and “FE” (Final Elution), each collected in a 10× volume in relation to the used input plasma volume.

The processed samples were then stored at −80° C. until downstream analyses. However, for biophysical characterization with TEM and NTA, an immediate buffer exchange to 1×dPBS was performed. This step was crucial for maintaining the structural integrity of the isolated EVs.

### Method Development and Optimization

In the method development and optimization phase, analytical techniques included TEM, NTA, and western blotting, in addition to LC-MS/MS-based proteomic analysis for a comprehensive EV characterization and method efficiency evaluation (FIG. 1).

Optimization of Resin-to-Plasma Ratio for Balanced Sample Purification Vs. EV Recovery

In both CC700-based EV pre-purification and Q-Sepharose-based EV fractionation steps, an investigation was performed to strike a balance between the efficiency of EV isolation (assessed using 1D-PAGE silver staining) and EV recovery (indicated by anti-CD9 western blot). For CC700, four resin-to-plasma (mg:μL) ratios were tested: 2.5, 3.0, 3.5, and 4.0. The comparative analysis (FIG. 7A) revealed that the 3.0 mg/μL ratio provided a moderate level of protein purification, especially for albumin and IgG, while maintaining a strong CD9 signal. Since this is the initial step and larger EV amounts are favorable for subsequent fractionation, the 3.0 mg/μL ratio was selected as an appropriate balance between the purity and recovery of EV isolates. Moving to the Q-Sepharose SAX fractionation step, after initial tests, two resin-to-plasma ratios were selected to evaluate and compare: 8.0 and 5.0. The acquired results (FIG. 7B) demonstrated that the 8.0 mg/μL ratio resulted in effective protein distribution across multiple fractions with strong CD9 signals present in the last two fractions, pH2, and FE, indicating a successful EV fractionation. On the contrary, at a ratio of 5.0 mg/μL, most of the proteins eluted in the FT fraction due to insufficient retention, and the EV recovery rate was much lower. We, therefore, selected a ratio of 8.0 mg/L in the SAX fractionation step.

### Assessment of Fractionation Performance Based on Plasma Protein Distribution Patterns

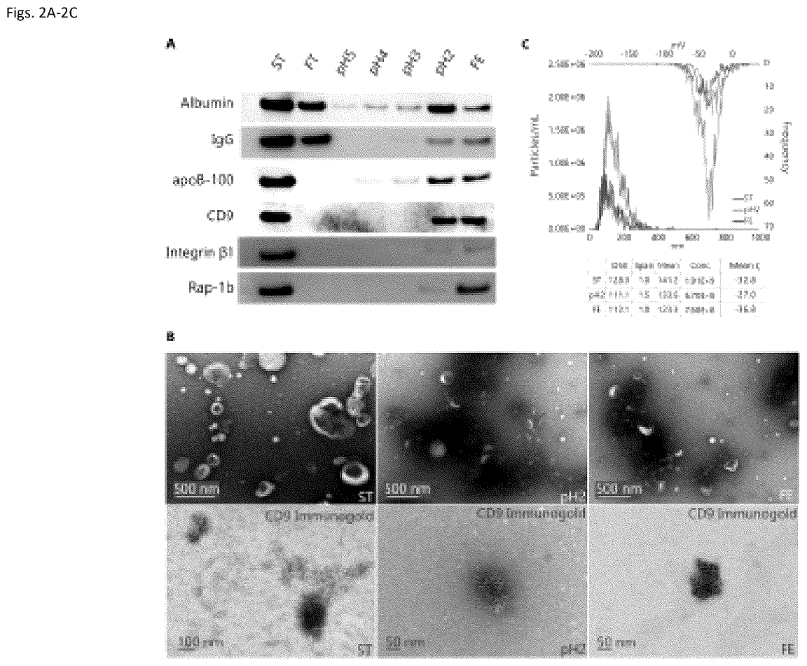

1D-PAGE followed by silver staining (FIG. 7B, ratio 8.0) and western blotting against three major plasma proteins, namely albumin, IgG, and apoB-100 (FIG. 2A) showed that the SAX-based technique resulted in efficient fractionation of the major plasma proteins in the EV-enriched pre-purified ST sample. These patterns demonstrated the method's ability to efficiently separate EV subpopulations and EVs from free plasma proteins and presumably other plasma components based on their charge. The method effectively enriches the residual top-abundance free plasma proteins in specific fractions, thereby decreasing the dynamic range within EV-rich fraction, an essential step for enhancing the depth, dynamic range, and quantitative accuracy of the downstream proteomic analysis. Contrary to aiming for the exhaustive depletion of predominant plasma proteins at the first step of EV enrichment using the multimode CC700 resin, this strategy seeks to establish a balance between (a) the fractionation efficiency in separating EVs from top abundance plasma proteins and separating EVs' subpopulations, and (b) EV recovery, which in turn, improves the performance of subsequent analytical readouts. Here are the observations made for several selected plasma proteins.

Albumin—Human serum albumin (ALB), which constitutes about 50-60% of total plasma protein¹³, displays a unique elution pattern (FIG. 2A). Upon analyzing the 1D-PAGE (˜66.7 kDa) and western blot bands, the highest observed intensity is present in the ST sample in both experiments, reflecting its status as the initial input. Similarly, both experiments showed substantial amounts of albumin were detected in the flow-through (FT) and the pH2 fraction, while significantly lower signals were detected in the pH5 to pH3 fractions. Albumin has an isoelectric point (pI) of 4.5-5.0¹⁴, which makes the initial elution in FT (pH ˜7) reasonable per the “2 pH unit rule” that is widely used in ion exchange chromatography¹⁵. The detection of an increased amount of albumin in the latter pH2 fraction might be indicative of albumin molecules that are bound to the surface of EVs, as proteins can exhibit altered binding behavior based on their interaction with EV surface molecules or incorporation into EV lumen or EVP structure as their cargole, 12. Albumin also showed a moderate band intensity in the final elution (FE) fraction. Distributing this most abundant plasma protein in several fractions narrows the dynamic range within each fraction, enhancing the potential for detecting less abundant proteins (e.g., EV-related proteins).

IgG-Another major plasma protein, IgG, contributes to approximately 10-20% of the total plasma protein¹³. Both the 1D-PAGE (˜50 kDa, IgG heavy chain) and western blot experiments displayed a clearly observable band in the FT fraction, denoting that the majority of IgG was efficiently eluted at this early stage, which is due to its higher pI value of 6.5-9.518. Interestingly, no bands were detected in the pH5 and pH4 fractions, but a faint signal in pH3 suggests the presence of a subset of IgG or IgG-related complexes, and the band signal becomes more visible in the pH2 and FE fractions. The trace amounts of IgG in the latter fractions suggest likely interactions with EVs or their distinct post-translational modifications, e.g., glycosylation, or incorporation in the EV structure as cargo¹⁹. With IgG largely eluting in the FT fraction, the protocol ensures that its residual amount does not compromise the detection of less abundant proteins in the subsequent analyses.

ApoB-100—Although apoB-100 is less abundant in plasma than albumin or IgG, it is a major constituent of low-density lipoproteins (LDL). Their close sizes to EVs usually cause ambiguities in distinguishing these two entities when using size-based methods like SEC²⁰. The apoB-100 western blotting presents a weak signal in the pH4 fraction, indicating the starting point of its elution, which is followed by gradually intensified bands in the later fractions. Meanwhile, the protein abundance observed in these bands is substantially lower, compared to the ST sample. This obvious difference shows the method's effectiveness in fractionating LDLs. ApoB-100's presence in late fractions with lower amounts might reflect potential interactions or co-elution of lipoproteins with EVs.

To summarize, based on the provided observations, the developed approach efficiently fractionated plasma constituents, including free proteins, which in turn reduces the dynamic range to potentially enhance the characterization of EV subpopulations. The enabled separation is particularly important for improving the depth of downstream proteomic analysis of EVs and EV subpopulations isolated from relatively small plasma volumes (i.e., 50-200 μL).

### Validation of EV Enrichment and Fractionation Based on Semiquantitative Assessment of Selected Proteins

To demonstrate the EV enrichment using the developed workflow, three notable EV-associated proteins were investigated: CD9, integrin β1, and Rap-1b. As per the MISEV (Minimal Information for Studies of Extracellular Vesicles) 2018 guidance¹, the chosen proteins are well-qualified as EV-associated markers due to their distinct roles and cellular localizations. CD9, a member of the tetraspanin superfamily, plays pivotal roles in EVs' biogenesis, cargo selection, targeting, and uptake processes, commonly serving as an EV surface marker²¹. Integrin 1, a membrane protein, is integral for cell adhesion and recognizes the extracellular matrix, making it a reliable indicator of the EVs' membrane components. Rap-1b, a cytosolic protein, is involved in intracellular signaling processes, further enhancing the diversity of proteins to represent various EV compartments. These three proteins, representing EV-specific tetraspanin, membrane, and cytosolic components, respectively, provide an extensive validation spectrum for the presence of EVs.

CD9-Western blot results for CD9 reveal a distinct and strong band in the ST sample, indicating its significant abundance in the starting material (FIG. 2A). Interestingly, post-fractionation, only the pH2 and FE fractions manifest strong bands for CD9. This selective elution underscores that the majority of EVs are particularly enriched within these two fractions, thereby validating the efficacy of the fractionation procedure.

Integrin β1—The presence of integrin 1 in the pH2 and FE fractions underlines its potential as a reliable marker for the verification of EV presence, particularly when complemented by other established EV markers (FIG. 2A). Its detection on the western blot shows a less intense signal compared to CD9 despite its plasma input volume being double that used for CD9. This difference may result from its relatively lower expression level in the parent cells or be ascribed to its more auxiliary role in EV biogenesis, as compared to CD9²².

Rap-1b—Cytosolic protein Rap-1b plays a crucial role in various cellular processes, including integrin activation and adhesion²³. Its detection in EV-containing fractions underscores the intricate cargo-sorting mechanisms inherent to EVs. As it is not a secreted protein, the detection of Rap-1b in the EV-containing fractions directly indicates its encapsulation within EVs, thus serving as evidence for efficient EV isolation and fractionation.

The notable variance in band intensity between the FE and pH2 fractions suggests a potentially selective enrichment of EV subpopulations with varied cargo content. This disparity may indicate the differential biogenesis pathways or cellular states from which these EVs originate. For instance, it is conceivable that EVs rich in Rap-1b might be released during specific cellular events, such as cell-to-cell signaling or stress response, given Rap-1b's involvement in cell adhesion processes.

In conclusion, the western blot analysis effectively validates the presence and charge-based fractionation of EVs and the three selected top abundance plasma proteins. The distinct fractionation patterns observed for these proteins confirm the method's efficiency in separating plasma components, including free proteins and EVs, based on electrostatic interactions. Instead of seeking the total removal of the main plasma proteins, this approach aims to achieve a balance between the recovery of EV subpopulations and the separation of EVs from the top plasma proteins, thus facilitating further analysis. The presented results of efficient charge-based fractionation establish a solid foundation for the subsequent proteomic analysis.

### Characterization of EV Morphology

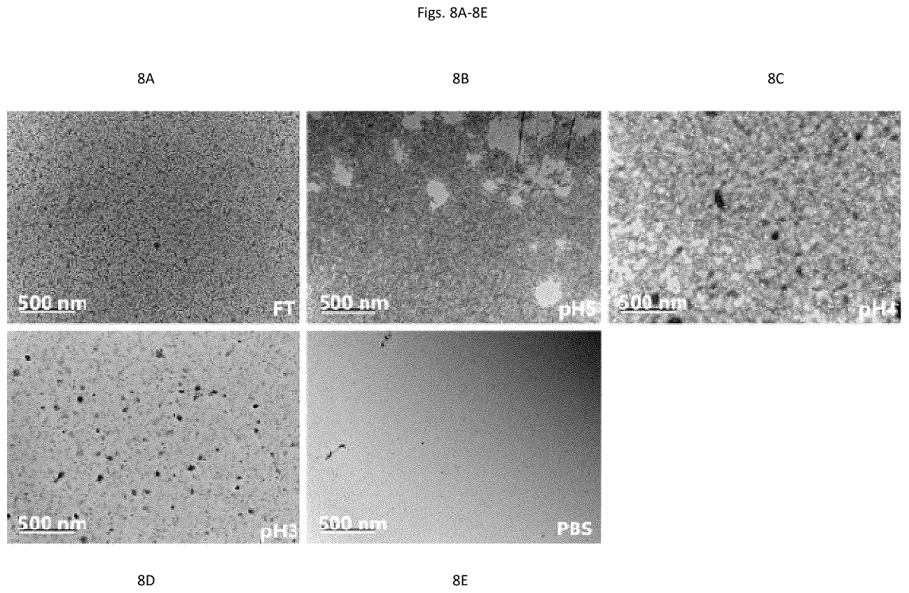

TEM serves as a crucial technique for visualizing the morphology of EVs. The TEM images (FIGS. 2B and 8A-8E) of the seven sample types (i.e., ST, FT, pH2, pH3, pH4, pH5, and FE fractions) and a reference blank dPBS demonstrate the presence of characteristic EV-like particles prominently in the ST sample, and pH2 and FE fractions. This observation, together with the western blot results shown in FIGS. 7A and 7B, further validates the effectiveness of the developed fractionation method.

Notably, these EV-like particles display a characteristic “saucer” or “cap” shape with a dented center. This unique morphology is a known property of EVs in TEM visualization²⁴. The phenomenon is due to the drying step during the preparation phase, which partially collapses the vesicles, leading to the formation of saucer-like structures.

The EV size spans from approximately 120 nm to 300 nm. Within the ST sample, certain instances of EV aggregation are evident, wherein multiple EV-like particles appear to cluster together. Such aggregation tendencies are commonly reported²⁵, often influenced by several factors, such as the proteins present on the EV surface and the specific conditions under which EVs are isolated or prepared.

Moreover, there are smaller, uniformly bright particles without the dented center present in the EV-containing samples. These particles, potentially representing co-eluted lipoproteins or non-vesicular extracellular particles (NVEPs) 2, further align with the present western blot observations, suggesting the co-enrichment of specific plasma components.

To augment the specificity of our TEM imaging and further validate the presence of EVs, an immunogold labeling technique targeting the CD9 protein was employed. The resulting TEM images for the EV-containing samples (ST, pH2, and FE) demonstrated the definitive binding of gold nanoparticles (10 nm) to the surface of the saucer-shaped particles. The detected immunoaffinity-based binding verified the presence of EVs and the efficacy of the chosen marker.

### Size Distribution and Zeta Potential of EVs

NTA delivers a detailed assessment of particle size distribution, concentration, and surface charge attributes. FIG. 2C presents a composite plot that includes an inset table, which concisely summarizes the key statistical measurements, elucidating the characteristics of particles in three EV-enriched samples ST, pH2, and FE.

In the plot depicting particle size distribution versus concentration, originating from the lower left corner, the ST sample is observed to have a median size (D50) of 128.0 nm. Comparatively, the pH2 and FE fractions exhibit slightly smaller median sizes of 111.2 nm and 112.1 nm, respectively. The particle size distribution in the ST and FE samples shows a span value (a metric indicating the width of the size distribution) of 1.0, signifying an intermediate breadth in the size distribution curve. Conversely, the pH2 fraction shows a slightly wider distribution with a span value of 1.5, and a discernible tailing peak on the plot.

The count-weighted mean sizes for the ST, pH2, and FE samples were measured to be 141.2 nm, 133.6 nm, and 123.3 nm, respectively. Additionally, the particle concentrations for these samples are recorded as 1.9E+9, 6.5E+8, and 7.5E+8 particles/mL, respectively. These data indicate a nominal particle loss, possibly because of EV lysis caused by the harsh conditions in the pH2 and FE fraction before the buffer exchange process.

In the upper right section of the plot, which represents zeta potential against frequency, the ST sample exhibits an average zeta potential value of −32.8 mV. In comparison, the pH2 and FE samples record mean zeta potentials of −27.0 mV and −36.8 mV, respectively. The increased negative charge in the FE sample relative to the pH2 sample aligns with the expected outcomes based on the mechanism of anion exchange chromatography heightened charge densities of EVs in the FE fraction contribute to stronger binding to the SAX resin. It also indicates charge-based EV subpopulations, aligning with western blot findings.

### Proteomic Data Analysis

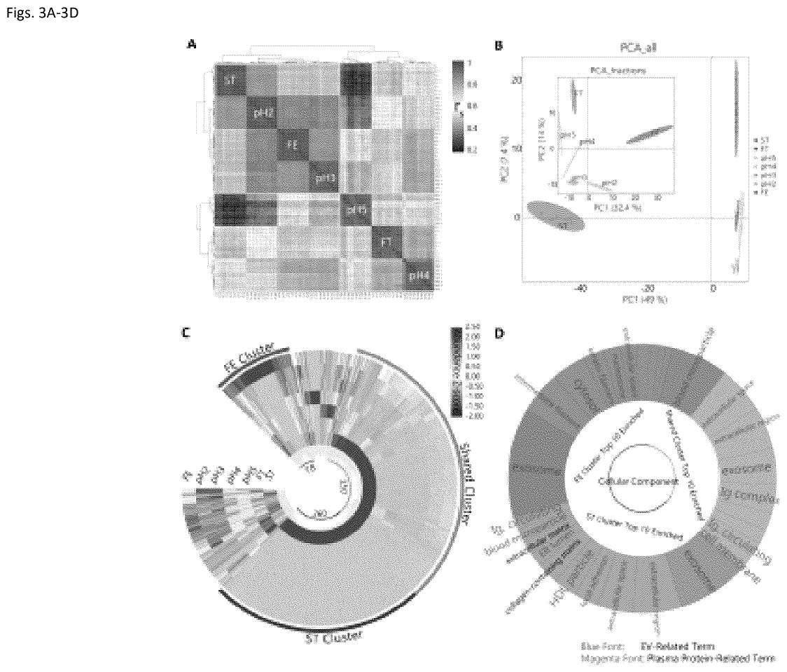

Advanced proteomic data analyses were crucial in characterizing the plasma-derived EVs (FIG. 3). The applied techniques included intercorrelation analysis and PCA, which not only demonstrated the reproducibility of our method but also suggested the relations among EV-containing samples. Gene ontology (GO) enrichment analysis of the distinct protein clusters from the hierarchical clustering provided insights into the enrichment of varied EV-related proteins and the depletion of plasma proteins within different EV-containing samples, highlighting the efficacy of our fractionation approach in isolating specific EV subpopulations. Moreover, the Venn diagram analysis was used to identify unique proteins in each EV fraction, shedding light on potential protein markers characteristic of distinct EV subpopulations.

### Intercorrelation Analysis

An intercorrelation analysis using Spearman's rank correlation coefficient (rs) was performed on the LFQ proteomic data to evaluate the method's reproducibility. Nine data sets derived from three preparation replicates and three LC-MS injection/analysis replicates for each sample type were subjected to the rs calculation, followed by a hierarchical clustering analysis. The heatmap generated (FIG. 3A), particularly along the main diagonal, indicated high quantitative reproducibility within each sample's data set, thereby confirming the method's reliability.

The unsupervised hierarchical clustering analysis revealed clear grouping patterns of samples based on quantitative proteomic profiles (FIG. 3A). The FE fraction initially demonstrated a close clustering and similarity with the pH3 fraction, and this combined FE-pH3 group subsequently clustered with the pH2 fraction. This trio was then grouped with the ST sample, finally forming a distinct dominant cluster. This FE-pH3-pH2-ST cluster was markedly separated from the other predominant cluster, which consisted of pH4-FT-pH5.

The arrangement within the hierarchical clustering suggests both similarities and differences in the proteomic profiles. Notably, the FE fraction did not directly cluster with pH2, implying the potential presence of distinct EV subpopulations. However, the formation of a dominant cluster encompassing ST, pH2, pH3, and FE suggests potential EV-like molecular features in the pH3 fraction, even though the specific molecular profiles have not been confirmed by other techniques. This observation proposes a subtle proteomic similarity and diversity among these fractions, providing a rationale to explore the pH3 fractions further in the pilot profiling experiments of clinical samples. The observed clustering not only underscores the uniqueness of the molecular composition in each fraction but also suggests a collective proteomic phenotype that could be instrumental in differentiating EV-rich fractions from other fractions with enriched high abundance free plasma protein species.

### Principal Component Analysis

PCA provides a holistic visualization of the proteomic profiles across samples. From the main “PCA_all” panel (FIG. 3B), the ST sample occupies a unique position along the PC1 dimension, which accounts for 49% of the total variance. This delineation underscores the distinct proteomic composition of ST when compared with the SAX fractions.

To further explore the fractions, the ST sample was removed for separate analysis. The results, as illustrated in the nested “PCA_fractions” panel, reveal a complex landscape. The FE fraction is located on the far right of the PC1 axis, which takes up 32.4% of the total variance, whereas the pH2 fraction appears around the midpoint. Their positions suggest distinct proteomic compositions and abundances, potentially correlating to different EV subpopulations.

The variance observed along the PC2 axis of the inset plot, representing 14% of the total variance, further distinguishes the proteomic profiles of the pH2 and FE fractions. This axis highlights differences in addition to those captured by PC1, especially emphasizing the differential nature of these two EV-enriched fractions. The elongated ellipses show 95% confidence for each sample. Notably, all nine data points corresponding to replicate experiments for a given sample group fall within their respective confidence ellipse, which is indicative of the reproducibility of the method. The clear separation between samples, particularly given the lack of overlap between ellipses, reflects their distinct variance patterns, reinforcing the efficacy of our fractionation method.

By linearly reducing the dimensionality of the original protein abundance data matrix, PCA allowed us to distill the multivariate data into principal components. This process reveals sample variability patterns that were not immediately discernible from the original proteomics data, facilitating a more straightforward interpretation of complex biological systems.

### Unsupervised Hierarchical Clustering

Utilizing unsupervised hierarchical clustering on the LFQ proteomic data demonstrated significant differences in protein abundances among the samples, which confirms the fractionation efficiency of the developed method. This analysis reveals distinct protein clusters characteristic of different samples (FIG. 3C). Two major clusters demonstrated by their highest abundance of proteins were identified. The first cluster encompasses 78 proteins most abundant in the FE fraction, labeled as ‘FE cluster’ bracketed by a pair of outer and inner red arcs (FIG. 3C). The increased number of IDs could be due to the reduced dynamic range of the FE sample and, therefore, a lower extent of ionization suppression than in the analysis of the ST sample, which in turn resulted in the increased profiling depth for the fractionated EV sample. The second, a considerably larger cluster, was predominantly abundant in the ST sample. However, a more detailed cross-sample (i.e., radial) inspection revealed that this vast cluster could be further dissected into two sub-clusters. The first of these sub-clusters displayed a moderate yet noticeable abundance in the FE and pH2 fractions, leading us to term it the ‘shared cluster’ (cyan arcs), which contains 250 proteins. Conversely, the second sub-cluster of 260 proteins, labeled as ‘ST cluster’ (blue arcs), was exclusive to the ST sample. These low-abundance proteins were not detected on the fractionated sample, possibly due to smearing across several fractions, which made their signal below the detection limit. A gene ontology enrichment analysis was performed, focusing on cellular component terms to gain deeper insights into the roles and potential significance of the proteins within these clusters.

### Gene Ontology Enrichment Analysis

The Krona pie chart (FIG. 3D) integrated the results of the individual GO enrichment analysis, providing visualization of the top ten most significantly enriched cellular component terms for previously identified FE, Shared, and ST clusters. Each term's area reflects its statistical significance as evaluated by the negative base 2 logarithm of the p-value.

ST Cluster. The ST cluster prominently features the term “exosome” as the largest sector, indicating efficient EV enrichment. However, alongside this dominating EV-associated term and several other EV-related broader GO terms (i.e., “blood microparticles,” “extracellular space,” “extracellular region”), terms were also identified that are indicative of secreted plasma proteins, including “high-density lipoprotein (HDL) particle” and “circulating immunoglobulin complex (lg, circulating).” This combination of GO terms reflects the higher complexity level of the ST sample before it was subjected to the subsequent SAX fractionation.

Shared Cluster. In the Shared cluster, the most significant term is “blood microparticle,” which was closely followed by the terms “extracellular space,” “extracellular region,” and “exosome.” EVs predominantly originated from blood cells and endothelial cells were likely responsible for the presence of proteins responsible for allocation to this cluster. Other GO terms in this cluster, such as the fourth-placed “exosome” and the tenth-placed “cell membrane,” prove the efficient EV enrichment in both the EV fractions and the unfractionated ST sample. The prominent sectors corresponding to the terms “immunoglobulin complex (lg complex)” and “lg, circulating” indicate the presence of antibodies in the studied samples. These results could be attributed to EVs' role as intercellular communicators, where they might harbor immunoglobulins, either as cargo or associated with their membranes (possibly due to the high abundance of immunoglobulins in blood and the so-called “sponge effect” 26), implicating possible immune interactions or modulations.

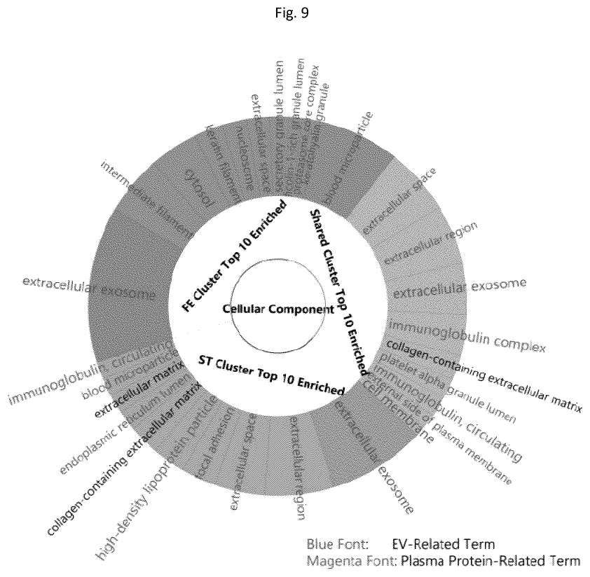

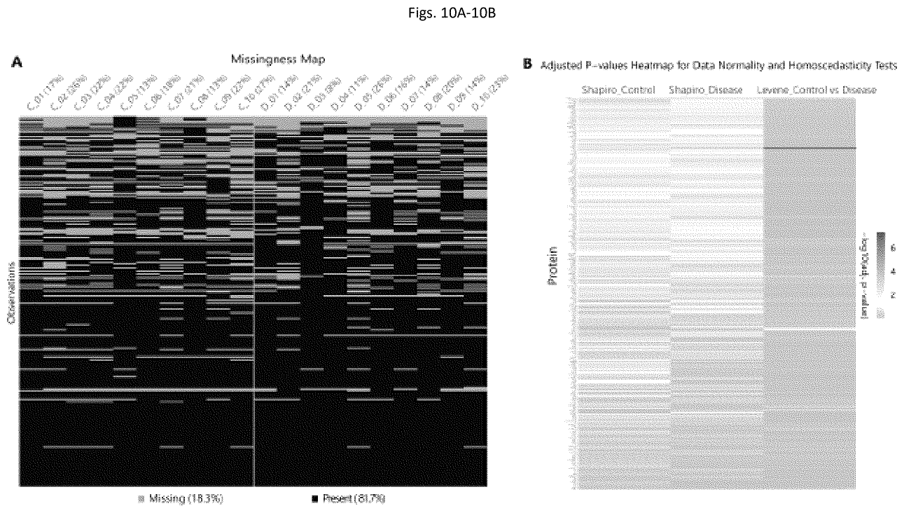

FE Cluster. Within the FE cluster, the term “exosome” once again dominates. However, the rest of the terms are more diverse. The presence of the “intermediate filament” underscores the cytoskeletal dynamics within EVs, perhaps hinting at their role in maintaining vesicular structure or at the specific biogenesis pathways. The terms “cytosol,” “nucleosome,” “proteasome core complex,” and other organelle-related terms further highlight the rich internal content of the EVs isolated in this fraction, emphasizing their role as carriers of diverse cargos (see the full version of Krona pie chart in FIG. 9).

In conclusion, the GO term enrichment analysis confirms the efficient EV enrichment and fractionation by the developed method. While demonstrating its predominant EV association, each cluster also offers additional insights into the diverse molecular composition of EVs and their subpopulations.

### Distinct Proteomic Profiles of EV Fractions

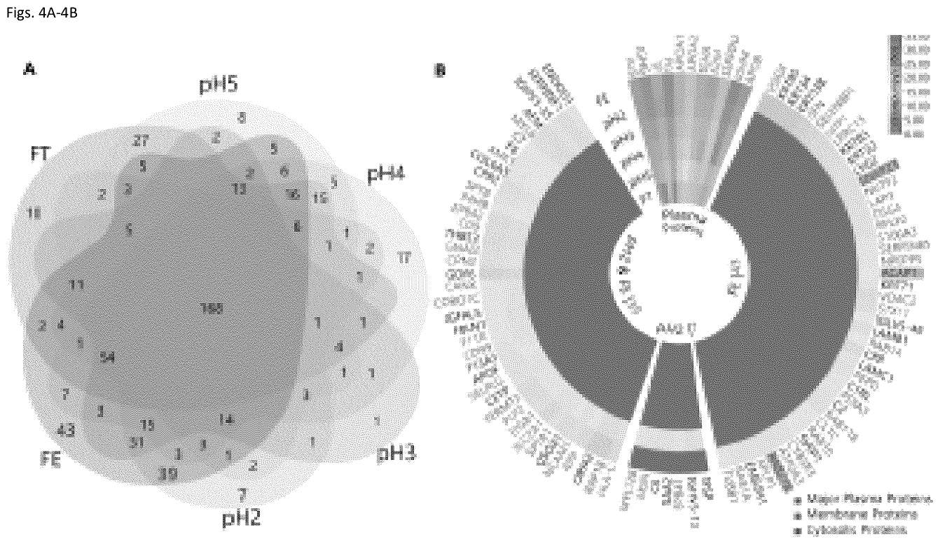

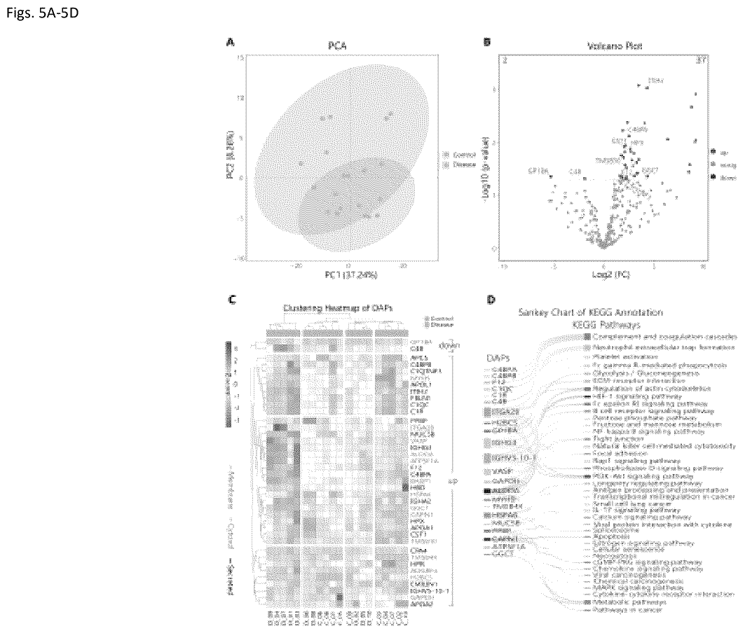

To further dissect the proteomic data, a detailed mapping was conducted of 11 plasma proteins and 89 uniquely detected proteins in EV fractions (FIGS. 4A-4B). A Venn diagram was first plotted (FIG. 4A) for all fractions to extract those proteins that are uniquely present in pH2 and FE or shared by the two fractions. As a result, 43 and 7 proteins were uniquely identified in the FE and pH2 fractions, respectively. This uniqueness could be attributed to the reduced dynamic range of the fractionated sample, allowing for a deeper proteomic exploration. Meanwhile, 39 proteins were commonly detected in FE and pH2 fractions. The relative abundance of these 89 proteins in total was subsequently illustrated in a circular heatmap (FIG. 4B).

The distribution of 11 plasma proteins was also mapped, including the most abundant albumin and IgG¹³, as well as apolipoproteins, in the same heatmap. The precise analysis of these proteins provided a clear perspective on their elution patterns in SAX-based fractionation. Albumin (ALB) and IgG (IGHG) were detected across all fractions at various levels, and their abundance distributions, along with apoB-100 (APOB), correlate well with the western blot results (FIG. 2A), reinforcing the consistency between the two orthogonal analytical techniques and emphasizing the reliability of the proteomic findings.

The subcellular origins of the 89 unique proteins was explored. Those originating from cell membranes or the cytosol, likely to be EV constituents, are highlighted in green and blue, respectively, to indicate their potential relevance to EVs. Within these color-coded groups, several proteins with shadowed font in the heatmap and discussed below stood out due to their association with intracellular membrane transport systems. These proteins are particularly involved in pathways such as endosomal maturation, which are reportedly linked to the formation of EVs, particularly influencing their biogenesis, intracellular trafficking, and the specific sorting mechanism of cargos⁴. Below are a few examples of such proteins.

SNAP23 is a member of the SNARE (Soluble N-Ethylmaleimide-Sensitive Factor Attachment Protein Receptors) protein family and plays a critical role in vesicle-membrane fusion processes. While its most established function is in mediating vesicle trafficking in non-neuronal cells, its involvement in the fusion of multivesicular bodies (MVBs) with the plasma membrane was recently reported²⁷. This fusion is an essential step in the secretion of exosomes, which are a subtype of EVs. Thus, SNAP23 may serve as a key player in ensuring the efficient release of exosomes into the extracellular environment. ACAP1 is an ArfGAP protein, which means it is a GTPase-activating protein for ADP-ribosylation factor (Arf) proteins. Arf proteins are small GTPases that play vital roles in vesicular trafficking. ACAP1 has been shown to regulate endocytic recycling by acting on Arf6, which is involved in endosome dynamics and the recycling of endocytic vesicles back to the plasma membrane²⁸. Given that the endocytic pathway is reportedly linked to the formation of MVBs²⁹ and the subsequent release of exosomes, ACAP1's involvement in regulating this pathway could imply a role in EV biogenesis. RAB27B is a member of the Rab family of small GTPases. Known as a late endosomal protein, it has a well-established role in the secretion of exosomes. By regulating the docking and fusion of MVBs to the plasma membrane, RAB27B is directly involved in the final steps of exosome secretion³⁰. Other RAB proteins in the heatmap, including RAB10, RAB11B, and RAB14, have also been linked to vesicular trafficking and may influence EV biogenesis by governing the movement and fusion of vesicles.

Understanding the roles of the above-discussed proteins in EV biogenesis and functions can provide insights into the complex and diverse molecular mechanisms that govern vesicle formation, trafficking, release, cell-cell communication, and involvement in pathologies.

### Evaluation of Method Applicability to the Analysis of Clinical Samples

The present method was used to obtain EV fractions from a cohort of age-matched clinical samples, comprising 10 PCa patients and 10 healthy age-matched controls. Utilizing the optimized nLC-MS/MS and data processing methods, LFQ data were acquired from the EV-enriched fractions, notably pH2 and FE, and also from the pH3 fraction, given its proximity to the pH2 fraction and its potential to isolate specific EV subpopulations. Further data analyses were divided into two directions. First, the LFQ data were consolidated from these fractions and then conducted a differential protein abundance analysis using the combined quantitative proteomic data. The goal was to determine differentially abundant proteins (DAPs) that might serve as distinct signatures that may be potentially indicative of PCa versus controls. Differential analysis was also conducted using the quantitative proteomic data for each individual fraction across all analyzed samples. Identification of differentially abundant proteins between PCa and control samples was performed.

### Selection of Differential Analysis Algorithm

For a small cohort of clinical samples, the combined LFQ dataset for the extent of data missingness was examined across the acquired datasets (FIG. 10A). With an average missing percentage of 18.3% sample-wise, this value is typical for proteome-scale datasets, especially for those derived from data-dependent acquisition-based LFQ analysis.

To maintain the integrity and reliability of the analysis, based on initial manual assessment, proteins that exhibited more than seven missing values within each group were filtered out. This approach ensured that each protein had a minimum of three valid values within each group, which allows for more reliable intra-group variance calculation for the differential analysis. Given the paired nature of the samples (age-matched disease and control donors), a conservative approach was taken by avoiding imputation of missing values. Imputation can inadvertently introduce artificial uniformity, potentially overshadowing actual differences in protein abundance that are critical in paired analysis.

Diving deeper into protein feature-wise attributes, the Shapiro-Wilk test³¹ was employed to gauge the data distribution normality within each group, coupled with Levene's test³² to access homoscedasticity (i.e., consistency of variances) between them. These tests are foundational, as the choice of differential analysis tools depends on data distribution. Applying an adjusted p-value threshold of 0.05, the heatmap from these tests (FIG. 10B) demonstrated that, for the majority of proteins, the log 2-transformed data maintained satisfactory levels of homoscedasticity. However, in terms of normality, only approximately half of the proteins conformed to the criterion. The remaining proteins exhibited distributions that were close to, but not fully consistent with, a normal distribution.

Given the data's characteristics, the LIMMA package in R was selected to conduct differential expression analysis. LIMMA, with its linear modeling foundation and empirical Bayes (EB) methodology, is particularly capable of efficiently processing complex datasets like ours. One notable strength of LIMMA is its robustness against deviations from normality. While traditional differential analysis might be heavily influenced by such deviations, LIMMA's empirical Bayes approach mitigates this issue by using strength across all features, providing more stable estimates³³. In the present case, the empirical Bayes method leverages information from the entire set of proteins, facilitating a more precise variability estimation. Consequently, even though our data is not perfectly normally distributed and there is only a moderate to small sample size, LIMMA can reliably discern differential expressions, making it an optimal choice for our dataset³⁴.

Comparison of EV Proteomic Profiles between Disease and Control Samples A PCA analysis was performed to visualize the multivariate data, to allow us to discern potential patterns that may justify further investigation. The PCA plot (FIG. 5A) indicates that the first principal component (PC1) accounted for 37.24% of the total variance, while the second principal component (PC2) explained an additional 8.26%. The ellipses on the PCA plot, corresponding to the 95% confidence intervals of the data points, indicate the variability within each group. For the Disease group, the ellipse is notably larger, which represents a higher variance in a diverse proteomic profile among patients. In contrast, the Control group displays a smaller ellipse, reflecting a more uniform distribution of quantitative proteomic profiles. Meanwhile, the overlapping region of the ellipses suggests a subset of proteins that are similar in their abundance in both groups. Taken together, these factors indicate the sophisticated mechanisms that shape the proteomes in both groups. The interplay between disease heterogeneity, individual biological variability, or even medical conditions highlights the great challenge of delineating disease states unequivocally. This complex interplay of shared and distinct proteomic patterns necessitates a deeper exploration of the biomolecular underpinnings of the disease.

Differential Proteomic Analysis of EVs Derived from PCa and Control Groups

Differential abundance analysis was conducted to pinpoint specific proteins, i.e., DAPs, which might play key roles in PCa manifestation, especially through the detection in EV populations. Then, the volcano plot was developed (FIG. 5B). This graphical representation compares the magnitude of change in protein abundance (log 2 fold change) with the statistical significance of the difference (p-value). A p-value of 0.05 and |log₂FC|>1 threshold was set for filtering. As a result, 37 upregulated and 2 downregulated proteins were identified as DAPs. Notably, several of these DAPs, including the ones briefly discussed below, have previously been associated with PCa progression.

A recent study suggests that ALDOA (fructose-bisphosphate aldolase A) plays a significant role in the progression of PCa. The research indicates that the ALDOA metabolism pathway could be a potential target for regulating PCa proliferation³⁵. This study provides insights into the importance of ALDOA in PCa's metabolic processes, suggesting its potential as a therapeutic target. Another study concludes that the coding variation in CPA4 (carboxypeptidase A4), may confer an increased risk of intermediate-to-high risk PCa among younger patients. This research indicated that certain genetic variations within CPA4 are associated with a more significant likelihood of developing more aggressive forms of PCa, particularly in younger individuals³⁸. Another study suggests that EGF receptor-mediated prostate tumor progression is dependent on STAT3, which regulates the expression of VASP (motility-limiting vasodilator-stimulated phosphoprotein), and the apoptosis nexus CASP3. This indicates that VASP may play a role in the migration and invasion processes of prostate carcinoma cells, contributing to the disease progression³⁷. Another relevant study suggests that in a PCa xenograft model, the loss of hormone dependence is marked by irreversible histological alterations associated with an expression of secretory MUC5B (mucin-5B) and other secretory mucins, MUC2 and MUC6, independent of the histological differentiation subtype³⁸. This indicates that MUC5B may play a role in the progression of hormone-refractory PCa. Other proteins, including GAPDH (glyceraldehyde-3-phosphate dehydrogenase), also show significant functions in cancer biology. One recent study showed that GAPDH is commonly upregulated in various types of cancer and potentially required for cancer cell growth and tumor formation³⁹. Additionally, while certain proteins did not emerge as DAPs based on our threshold, they hovered close to the threshold limit, suggesting their potential relevance. Among them, one protein, YWHAZ (14-3-3 protein zeta/delta), deserves special mention. A recent study used integrative tissue-omics to identify high YWHAZ expression subgroups, correlating with a high risk of disease progression, making YWHAZ a molecular biomarker associated with aggressive PCa⁴⁰.

In conclusion, proteomic analysis of liquid biopsy-based EV isolates provides valuable insights into the protein landscape of PCa. The identification of DAPs, coupled with the validation from existing literature, underscores the robustness of our findings.

### Hierarchical Clustering Analysis of DAPs

Unsupervised hierarchical clustering analysis of the identified DAPs offers insights into the molecular alterations, which can be potentially associated with the disease (FIG. 5C). The row-wise analysis (protein-centric) of the generated heatmap revealed a distinct clustering pattern, reflecting the complex regulation of protein expression in response to disease, at least to some extent based on this proof-of-concept studies. A smaller cluster of downregulated proteins GP1BA (platelet glycoprotein Ib alpha chain) and C4B (complement receptor type 1) was clearly separated from a larger cluster of upregulated proteins, highlighting a significant difference in protein abundance between control and disease conditions.

Within the upregulated proteins, the heatmap further revealed three distinct subclusters, each representing unique protein groups with potential implications in the disease mechanism. The upper one was predominantly composed of extracellular secreted proteins (black fonts), suggesting a role for these proteins in extracellular signaling and interactions that are possibly linked to the disease's pathophysiology.

The middle cluster, enriched with cytosolic proteins (blue fonts), including VASP, ALDOA, ATP5F1A, HSPA6, GGCT, CAPN1, and TMSB10, as well as two membrane proteins (green fonts) ITGA2B and BASP1, indicates alterations in cellular processes or pathways. These proteins, together with those in the lower cluster with cytosolic TMSB4X, H2BC5, GAPDH, and the membrane protein ADGRF4, are indicative of their origin from EVs, adding a layer of our understanding of EV's involvement in the disease.

On the other hand, the column-wise clustering (sample-centric) demonstrates a complex interplay of factors influencing protein abundance levels that do not completely separate the disease and control groups. Notably, the first cluster is comprised exclusively of five disease samples, which share the highest expression levels of these DAPs, suggesting a potential subset of the patient-derived samples characterized by a unique protein expression signature. The second cluster represents a mix of two disease and four control samples, with the second highest level of DAPs' abundance, indicating that specific control samples might share expression patterns with the disease subset. The third cluster includes a singular control sample C_09, along with three disease samples, occupying an intermediate position in terms of DAP expression levels. Finally, the fourth cluster is entirely composed of five control samples, which exhibit the lowest expression of the DAPs, possibly delineating a protein expression profile characteristic of the absence of disease.

These results suggest that while there is a differential expression of proteins associated with the disease, there is also considerable overlap between control and disease samples in the acquired data, potentially due to the heterogeneity of the disease, individual biological variability, variability in the disease progression and manifestation, and possibly insufficient depth of proteomic profiling based on the used here sample amount and current LC-MS technologies. The presence of disease-only and control-only clusters may reflect specific pathophysiological states or subtypes within the broader disease category. In contrast, the mixed clusters could indicate that the disease process is not uniform across all patients. Additionally, the absence of age-related patterns within the clusters may imply that age is not the primary factor driving the proteomic differences observed, or it may be overshadowed by the disease's impact or the biological variability among individuals. However, the number of samples used in this pilot study was limited, which does not allow us to draw significant conclusions based on the age or disease status.

### Mapping Differential Proteomic Profiling to Transcriptomic TCGA Data

For verification of the present proteomic analysis, DAPs identified in the present study were compared against the publicly available transcriptome data from The Cancer Genome Atlas (TCGA)⁴¹. Through the UALCAN portal^{42, 43}, TCGA's repository of PCa were accessed to investigate the transcriptional profiles corresponding to these DAPs.

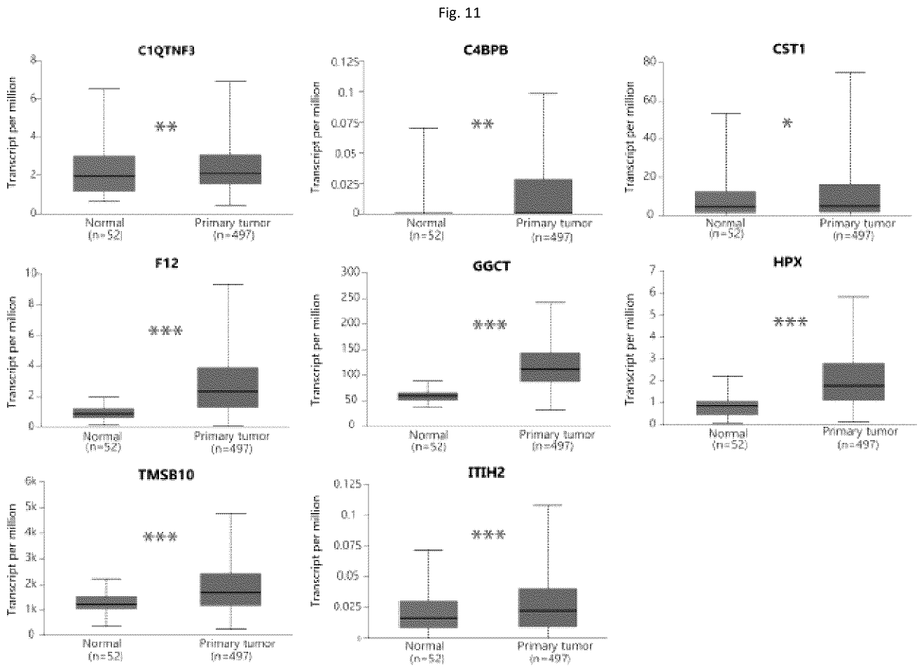

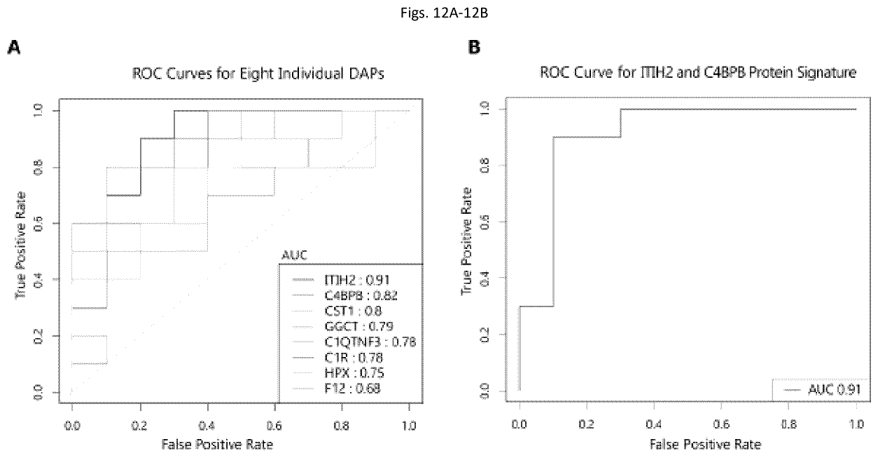

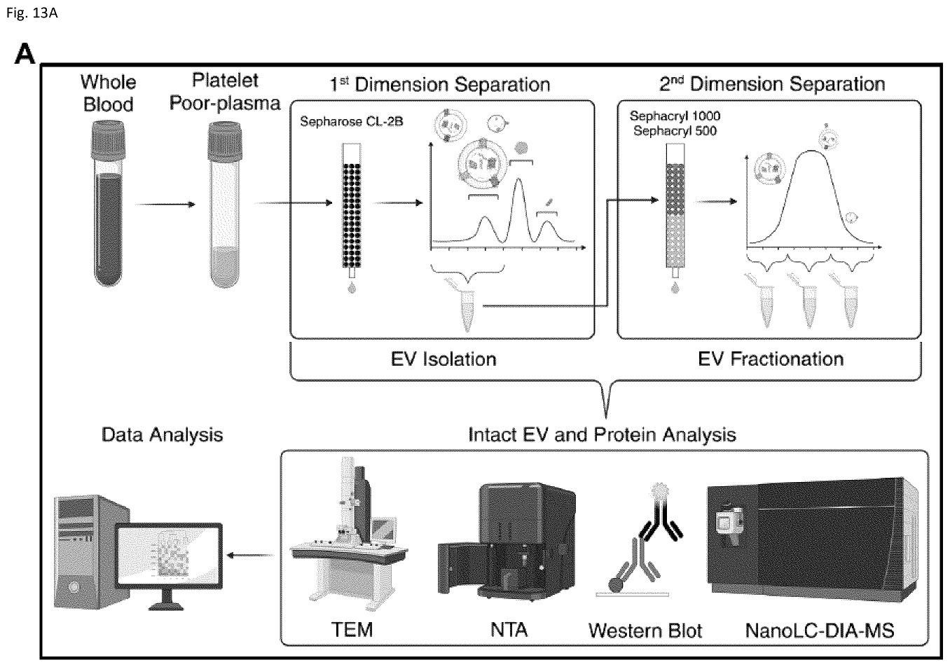

A subset of these proteins, including C4BPB, GGCT, F12, ITIH2, CST1, HPX, TMSB10, and C1QTNF3, demonstrated significantly upregulated transcriptional patterns in the TCGA dataset (FIG. 11). The correlation between our proteomic findings and the TCGA transcriptional data provides an additional layer of validation for the eight DAPs. This correspondence suggests that these proteins play a notable role in PCa and could be further investigated as potential biomarkers or therapeutic targets. The alignment of our results with TCGA data underscores the potential relevance of these proteins in the context of PCa. It also suggests that the combined multi-omic analysis of proteomic and genomic data can be a powerful approach in the ongoing effort to understand the complexity of cancer biology and to develop more effective cancer diagnostic techniques and treatments.

Receiver operating characteristic (ROC) curves of these eight selected DAPs were plotted for their differential abundance as potential diagnostic biomarker candidates upon appropriate additional validation (FIG. 12A). Interestingly, ITIH2 shows excellent diagnostic accuracy with an area under curve value (AUC) of 0.91. C4BPB and CST1 demonstrated promising performance, with AUCs of 0.82 and 0.80, respectively. GGCT, C1QTNF3, C1R, and HPX display fair to good efficacy, with AUCs between 0.75 and 0.79. F12, with an AUC of 0.68, shows limited discriminative power, highlighting its limited utility as a standalone biomarker. In addition, an ROC curve for a signature protein set from those with AUC >0.80 (i.e., ITIH2 and C4BPB) shows a combined AUC of 0.91 (FIG. 12B). These proteins may be considered for future validation and careful monitoring in potential follow-up studies using larger sample cohorts.

### KEGG Pathway Annotation of DAPs in Cancer Biology

Proteins serve as critical mediators of cellular function and are central to the processes that govern cancer progression and tumorigenesis. Examining the pathways in which these proteins are involved provides a broad view of the roles that these proteins may play within the complex network of oncogenic pathways (FIG. 5D). This approach is instrumental in elucidating the multifaceted interactions and processes that underlie cancer development and progression. Below, C4BPB, F12, and GGCT DAPs and the related protein-protein interaction pathways and their relevance to PCa biology are discussed based on reported publicly available TCGA and KEGG data.

C4BPB (C4b-binding protein beta chain) and F12 (coagulation factor XII) proteins both play roles in the complement and coagulation cascade pathway of the immune system. These proteins, crucial to immune response and inflammation, are highly relevant to tumor biology. The immune system, particularly the complement system, is a double-edged sword: while it is essential for neutralizing foreign threats, its anomalies can inadvertently aid tumor growth. This ambiguous role of the complement system in oncology has been reported⁴⁴. Recent studies explored the polyphosphate-Factor XII (F12) pathway in the specific context of PCa-associated thrombosis. The research revealed that this pathway is an active participant in cancer progression. In PCa, the polyphosphate-Factor XII was identified as a key driver of thrombotic events^{45, 46}. This association emphasizes the pathway's potential as a therapeutic target, offering avenues to mitigate thrombosis risk while addressing tumor growth and spreading.

GGCT (gamma-glutamylcyclotransferase), a key enzyme in the gamma-glutamyl cycle, is significant in cancer metabolism and therapy resistance. Identified within KEGG's metabolic pathways, its role extends beyond maintaining cellular redox balance, directly influencing tumor drug resistance⁴⁷. Further, based on the GGCT's direct contribution to tumor proliferation, inhibiting GGCT, particularly in GGCT-overexpressing tumors, including PCa, presents a promising therapeutic strategy⁴⁸. This dual functionality of GGCT, from metabolic regulation to influencing tumor growth and therapy response, highlights its potential as a therapeutic target. Its enzymatic activity, especially in the context of overexpression, offers a unique vulnerability in cancer cells, proposing a potential shift in treatment approaches.

Building upon these insights into the DAPs mapped to KEGG pathways, other proteins were explored that, though not directly associated with established pathways, display significant relevance in cancer progression based on the reported TCGA data. The observed overexpression of ITIH2 (inter-alpha-trypsin inhibitor heavy chain H2) in PCa underscores a complex interplay of molecular and cellular mechanisms. ITIH2, known for its involvement in stabilizing the extracellular matrix (ECM), may reflect critical adaptations within the tumor microenvironment, potentially facilitating structural changes conducive to tumor growth and metastasis⁴⁹. Intriguingly, the correlation between ITIH2 and estrogen receptor expression opens avenues for deeper exploration, particularly in PCa, where hormonal dynamics are pivotal⁵⁰. Given the role of estrogens in men, often altered significantly in PCa either due to the disease's hormonal manipulations (such as androgen deprivation therapy) or the tumor's local synthesis of estrogens via aromatase, ITIH2 overexpression might be an unforeseen consequence of these hormonal shifts^{51, 52}.

### Differential Proteomic Analysis of Individual Fractions

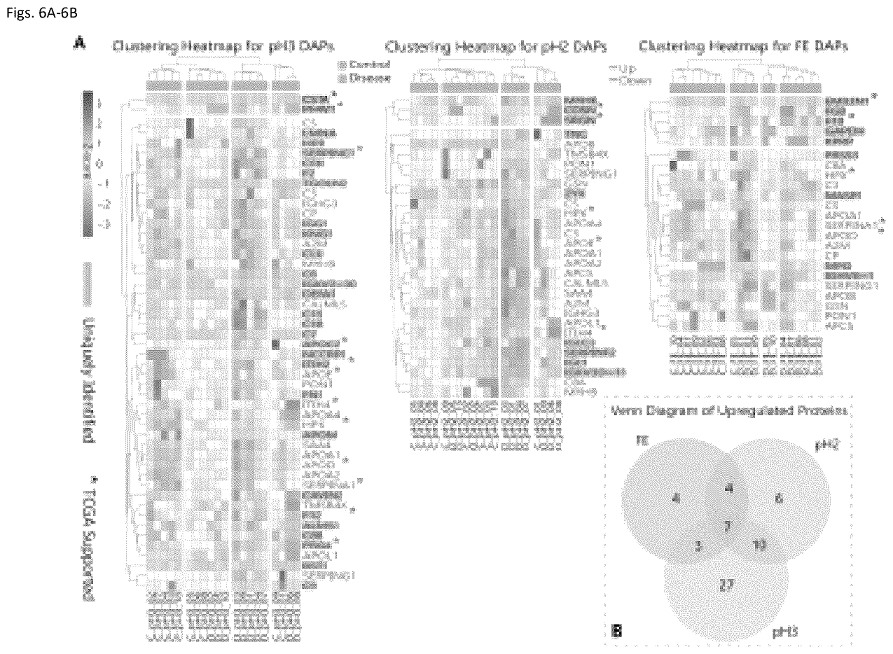

Applying uniform data analysis procedures and criteria across the three individual EV-rich fractions resulted in the determination of differentially represented proteins in PCa and control samples. The number of DAPs identified were as follows: FE (18 upregulated, 5 downregulated), pH2 (27 upregulated, 3 downregulated), and pH3 (47 upregulated, 2 downregulated) (FIG. 6A). Notably, all downregulated proteins were uniquely identified within their respective fractions. Among these, CSTA and PF4V in the pH3 fraction, CDSN and SRGN in the pH2 fraction, and EMILIN1 and F10 in the FE fraction exhibited expression patterns consistent with significant differences observed in their corresponding TCGA transcriptome data.

In the analyses of upregulated proteins, a Venn diagram was used to elucidate the unique and overlapping DAPs across the three fractions (FIG. 6B). This analysis revealed seven proteins, A2M, C3, PON1, APOA1, C5, SERPING1, and HPX, as common across all fractions, with HPX's expression corroborated by TCGA data and previously identified in the differential analysis of combined data. Specifically, the FE fraction uniquely presented four proteins: MPO, IGHV6-1, MASP1, and PRSS3. The pH2 fraction was distinguished by six unique proteins: ZYX, SERPINF2, IGLC3, TNC, IGL1, and IGKV3D-11. In the pH3 fraction, 27 unique proteins were identified, including PRG4, SERPINC1, ITIH2, APOC2, and F12, which aligned with the upregulation of the corresponding genes in PCa patients reported in TCGA data; notably, ITIH2 and F12 were also identified in the data analysis for combined fractions.

The present technology introduces methods to isolate EVs and fractionate EV subpopulations from biological samples such as human plasma using dual function chromatography, preferably including strong anion exchange fractionation. Unlike earlier methods, the use of anion exchange makes use of the electrostatic properties of EVs. One notable advantage of the technology is its effectiveness in reducing contamination, such as contamination from free plasma proteins, a common issue in EV isolation. Moreover, the present technology makes it possible to examine charge-based subpopulations of EVs, providing insights into the biomolecular compositions that may indicate diverse physiological or pathological conditions of the donors. The present fractionation methods can be effective in narrowing down the dynamic range of proteins in EV fractions, enabling deeper proteomic profiling. The fractionation methods are advantageously combined with LC-MS/MS-based proteomics for the molecular characterization of EVs. Using advanced LC-MS and data acquisition techniques, e.g., Orbitrap Astral or timsTOF mass spectrometers, and data-independent acquisition (DIA), can increase the depth of proteomic profiling of EVs. Such methods also enable higher throughput analysis while increasing the coverage of proteomic data and/or by using a larger volume of plasma (e.g., 200-300 μL) subjected to EV isolation and fractionation. Moreover, while the available prostate-specific antigen (PSA)-based tests are of poor specificity in the detection of prostate cancer, investigating the levels of post-translational modifications and proteoforms of such proteins as PSA and prostate-specific membrane antigen (PSMA) in plasma combined with EV subpopulation-based profiling can provide more specificity and sensitivity in diagnostic and prognostic applications. Additionally, a combination of proteomic and transcriptomic readouts, e.g., quantitative profiling of extracellular RNA, can be performed in EV subpopulations. Further analytical techniques such as western blotting, TEM, and NTA also can be employed in conjunction with the present fractionation methods.

### Example 1 References

1. Kostas, J. C., Greguš, M., Schejbal, J., Ray, S. & Ivanov, A. R. Simple and Efficient Microsolid-Phase Extraction Tip-Based Sample Preparation Workflow to Enable Sensitive Proteomic Profiling of Limited Samples (200 to 10,000 Cells). Journal of Proteome Research 20, 1676-1688 (2021).
2. Perez-Riverol, Y. et al. The PRIDE database resources in 2022: a hub for mass spectrometry-based proteomics evidences. Nucleic Acids Research 50, D543-D552 (2022).
3. Chen, C. et al. TBtools-II: A “one for all, all for one” bioinformatics platform for biological big-data mining. Molecular Plant 16, 1733-1742 (2023).
4. Sherman, B. T. et al. DAVID: a web server for functional enrichment analysis and functional annotation of gene lists (2021 update). Nucleic Acids Research 50, W216-W221 (2022).
5. Fonseka, P., Pathan, M., Chitti, S. V., Kang, T. & Mathivanan, S. FunRich enables enrichment analysis of OMICs datasets. Journal of Molecular Biology 433, 166747 (2021).
6. Chandrashekar, D. S. et al. UALCAN: A Portal for Facilitating Tumor Subgroup Gene Expression and Survival Analyses. Neoplasia 19, 649-658 (2017).
7. Chandrashekar, D. S. et al. UALCAN: An update to the integrated cancer data analysis platform. Neoplasia (New York, N.Y.) 25, 18-27 (2022).

### Example 2. Isolation, Enrichment, Fractionation, and Characterization of EVs by Methods Involving Size Exclusion Chromatography

To overcome the challenges that UC-based techniques create, the inventors employed the use of three different size-exclusion chromatography resins, Sepharose CL-2B, Sephacryl 500, and Sephacryl 1000, that have various chemical and mechanical properties, such as their chemical moieties and pore sizes. The use of Sepharose CL-2B to isolate EVs by SEC was disseminated in 2014, and since then, the resin has become widely used in EV isolation^{33,43}. However, Sepharose CL-4B and CL-6B seem to be gaining wide applicability in EV isolation as well40. Sepharose CL-2B contains 2% cross-linked agarose-based beads with an exclusion limit of 75 nm^{43,44} Sephacryl 500 and Sephacryl 1000 are comprised of allyl dextran and N-N′-methylene bisacrylamide with exclusion limits of 42 nm and 400 nm, respectively^{43,44}. Particle bead properties are summarized in Table 1, adapted from (Monguio-Tortajada, M., et al.⁴³). Sephacryl 500 and Sephacryl have dextran fractionation ranges between 40-20,000 kDa and 500-100,000 kDa, respectively. While the use of Sephacryl resins remains not as common as the Sepharose resins for EV preparations, there were several studies since the 1980s that demonstrated how useful Sephacryl resins can be for separation of nanoparticles, including liposomes, viruses, and outer-membrane vesicles from Gram-negative bacteria⁴⁵-48. It was also reported that Sephacryl 1000 had been used to isolate plasma-derived EVs, but only after a differential centrifugation step was performed^{49,50}.

A bilayer column consisting of Sephacryl 500 and Sephacryl 1000 was used to isolate EV subpopulation-enriched fractions from human blood plasma. By layering the two Sephacryl resins within an SEC column, the total effective fractionation range is extended to 40-100,000 kDa, allowing for the collection of subpopulations of smaller NVEPs and larger EVs in different fractions without losses, whereas the conventional use of one resin type limits the fractionation range.

TABLE 1
Size-exclusion chromatography bead properties reported for the separation
of branched dextrans. Sepharose CL-2B and Sephacryl 500 are similar in
terms of fractionation range and exclusion limits. The addition of Sephacryl
1000 as the top layer allows both the fractionation range and exclusion
limits to increase by an order of magnitude. This allows for effective
separation of the heterogenous EV isolate generated by 1D-SEC.

| | Matrix | Particle | Fractionation | Exclusion | Exclusion |
|---|---|---|---|---|---|
| Resin | Type | size (μm) | Range (kDa) | Limit (kDa) | Limit (nm) |
| Sepharose | 2% Cross-linked | 60-200 | 100-20,000 | 40,000 | 75 |
| CL-2B | Agarose | | | | |
| Sephacryl | Spherical allyl | 25-75 | 40-20,000 | 20,000 | 42 |
| 500 | dextran and | | | | |
| | N,N′-methylene | | | | |
| | bisacrylamide | | | | |
| Sephacryl | Spherical allyl | 40-105 | 500-100,000 | 100,000 | 400 |
| 1000 | dextran and | | | | |
| | N,N′-methylene | | | | |
| | bisacrylamide | | | | |

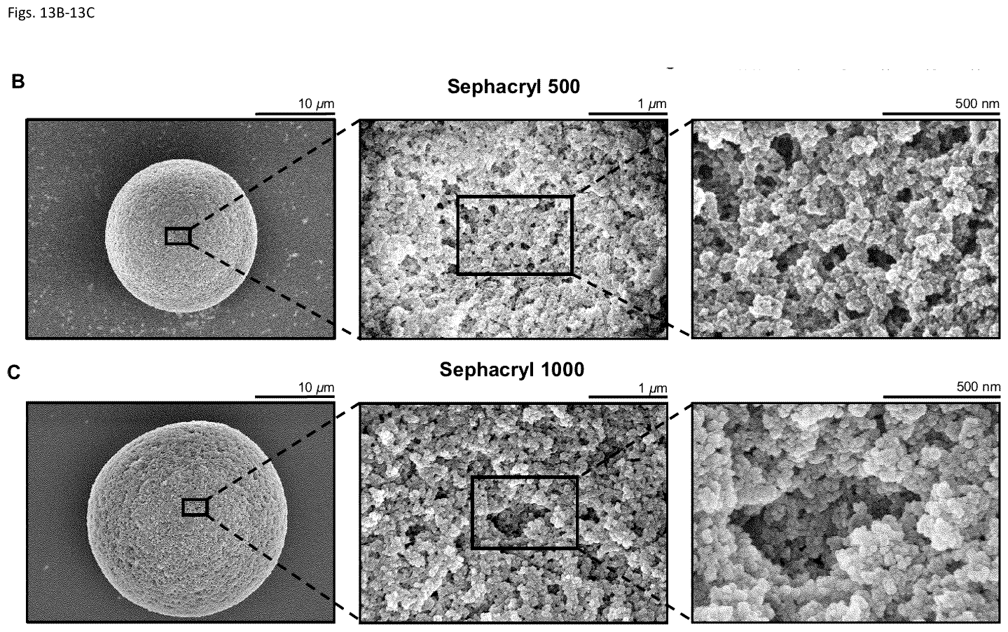

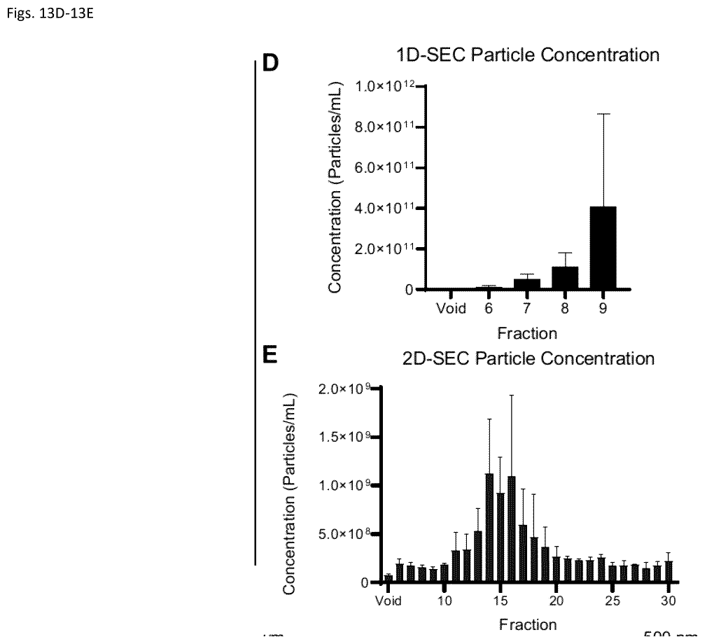

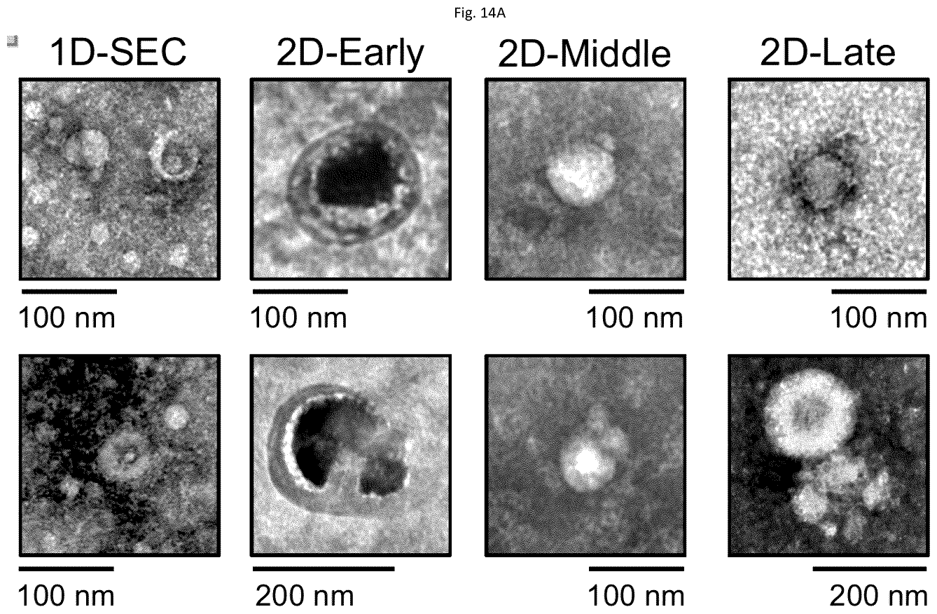

Within this study, the inventors assessed EV isolation from healthy control human blood plasma by one-dimensional (1D)-SEC, using Sepharose CL-2B, followed by a second dimension (2D)-SEC step, using a bilayer column consisting of Sephacryl 500 and Sephacryl 1000, in order to fractionate EVs into subpopulation-enriched fractions (FIG. 13A). Multidimensional separation methods have been used for a wide range of complex mixtures, including complex proteomes⁵¹ and monoclonal antibody protein digests⁵², whole cell lysate digests, and small molecules such as oils, steroids⁵³, and metabolites⁵⁴. A benefit of 2D-liquid chromatography (2D-LC), which can be used offline or coupled online, is that it increases the peak capacity of the system to allow separation of highly complex mixtures and also has the potential to resolve analytes that co-elute with each other enabling deeper proteomic profiling⁵⁴. The inventors then used the developed method to assess clinically relevant samples from patients diagnosed with prostate cancer (PCa) to probe for PCa or tumor-related proteins that are carried by the circulating EVs with the purpose of exploring EV cargo proteins that may potentially be more specific in identifying PCa using liquid biopsies compared to a traditional PSA test. While 1D-SEC has been shown to be sufficient for EV isolation^{33,55,56}, a further SEC separation step can offer enrichment of EV subpopulations, which can be beneficial to determining the EV cargo and understanding its role in health and disease.

### Methods

### Ev Isolation One-Dimensional Size-Exclusion Chromatography

An empty affinity chromatography (AC) column (Cat. #004212, Biocomma; Shenzhen, China) was packed with 5 mL of Sepharose CL-2B to a bed height of 8 cm, as described previously⁵⁶. Prior to packing, a hydrophilic filter (8.3 mm diameter, 1.6 mm length, 50 μm pore size (Cat. #ACF-083-16-50-1, Biocomma)) was placed at the bottom of the column, acting as a retaining frit. The column was washed with 10 mL of 1×dPBS. For all experiments, 150 μL platelet-poor plasma (PPP) was diluted with 75 μL dPBS (2:1) and loaded onto the column. The column was placed onto an Automatic Fraction Collector (AFC, iZon Science Ltd; Christchurch, New Zealand), and analytes were eluted with 1×dPBS. After the void volume of 1.2 mL, EV-containing eluate was collected in four 200 μL fractions. These fractions were subsequently pooled together to be one EV sample and concentrated to ˜30 μL using a centrifugal filter (30 kDa MWCO, Amicon, United States). This EV enrichment method is referred to herein as 1D-SEC.

### EV Fractionation by Two-Dimensional Size-Exclusion Chromatography

A hydrophilic filter was placed at the bottom of an empty AC column and was packed with 2.5 mL of Sephacryl 500 to a bed height of 4 cm and allowed to settle. On top, 2.5 mL of Sephacryl 1000 (bed height of 4 cm) was added for a total bed height of 8 cm. The column was washed with 10 mL 1×dPBS. Thirty μL of a concentrated EV isolate generated by 1D-SEC was loaded onto the column. After the void volume of 1.2 mL, EV-containing fractions were collected in 200 μL fractions. The first four EV-containing fractions were pooled together and are subsequently referred to as “2D-Early”, followed by pooling the next four EV-containing fractions, which are referred to as “2D-Middle”, and finally pooling the last four EV-containing fractions, which are referred to as “2D-Late”. All pools were concentrated to ˜30 μL using a centrifugal filter (30 kDa MWCO, Amicon, United States). This is referred to herein as 2D-SEC.

### Assessment of Sephacryl Stationary Phases for EV Enrichment

To validate the developed EV isolation method using 2D-SEC columns, the inventors utilized pooled PPP obtained from self-declared healthy male donors. The process began with the loading of 150 μL of PPP onto a 1D-SEC column containing Sepharose CL-2B. The EVs purified from this initial step were subsequently loaded onto a second-dimension SEC column comprising Sephacryl 1000 and 500. Three EV-enriched subpopulation fractions 2D-Early, 2D-Middle, and 2D-Late, which were named based on their elution order, were collected from the second dimension and characterized using nanoparticle tracking analysis (NTA), transmission electron microscopy (TEM), western blotting, and nano-liquid chromatography coupled to data-independent acquisition mass spectrometry (nanoLC-DIA-MS) (FIG. 13A). To visually inspect the surface morphology, particle size, and pore size of the Sephacryl resins, SEM imaging was employed (FIGS. 13B-13C). Imaging at the single particle level allows for the assessment of the surface morphology. Each particle appears nearly spherical with no visible damage or aberrations from the manufacturing process, with particle diameters ranging between ˜20-25 μm. It can be observed that Sephacryl 1000 has larger cavities throughout its surface compared to Sephacryl 500, as expected. Upon zooming into the surface of the beads, the difference in pore size becomes apparent. Sephacryl 500 is observed to have pore sizes of ˜50-100 nm in diameter, and Sephacryl 1000 is observed to have pore sizes between 500-1000 nm in diameter.

### Characterization of Intact EVs in Individual Fractions Following One- and Two-Dimensional SEC Separation

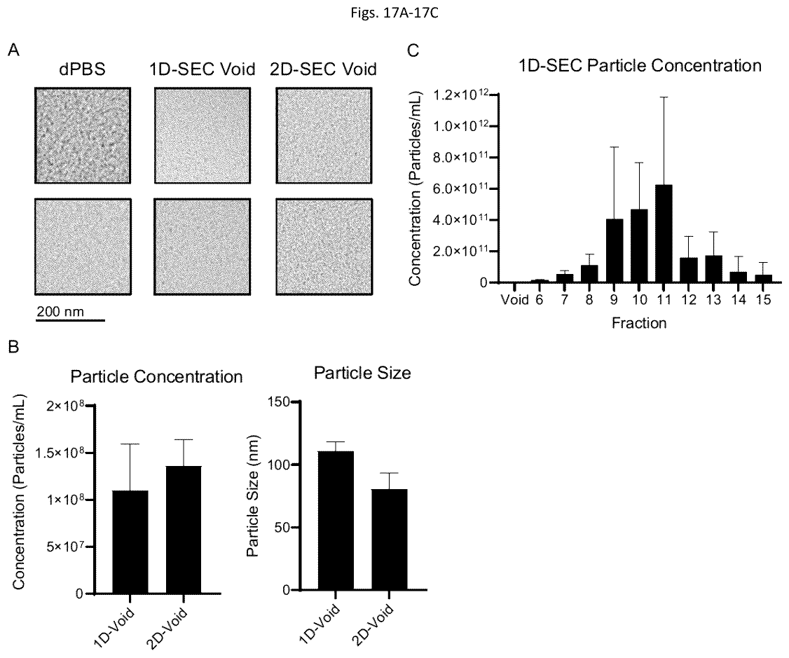

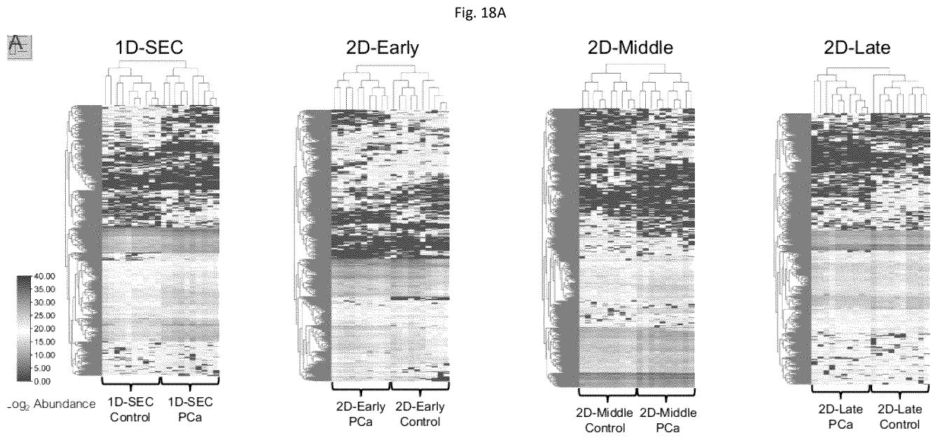

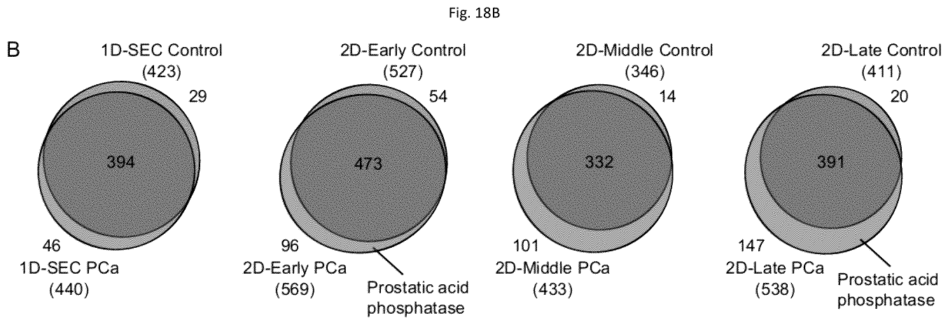

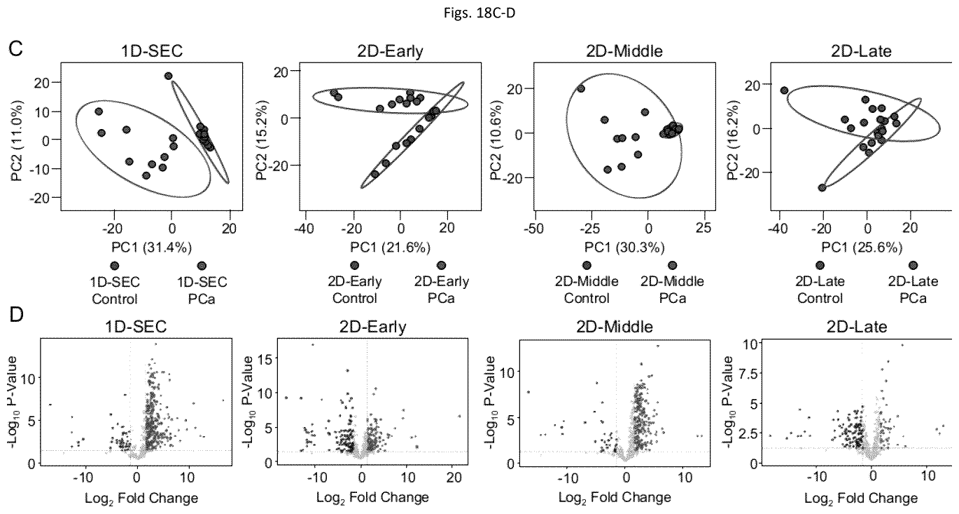

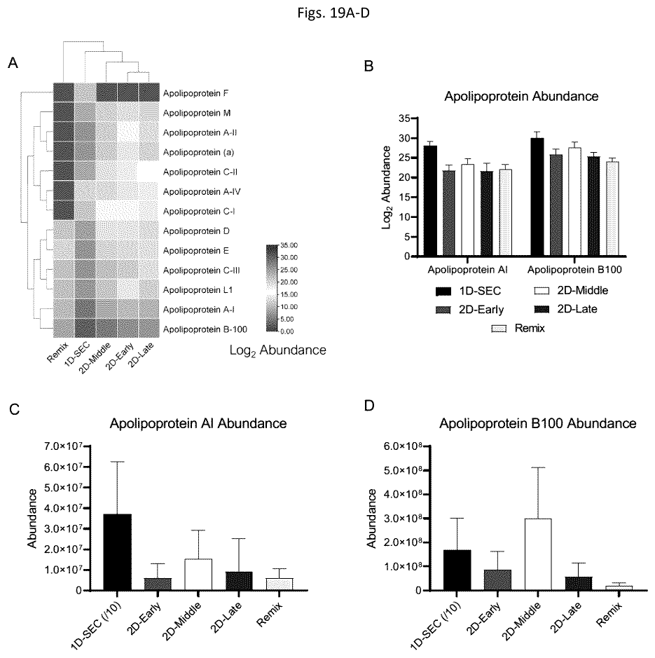

Quantitative analysis of intact particles was performed by NTA (FIGS. 13D-13E), N=3. Fractions consisting of 200 μL eluent each were collected and analyzed for particle concentration to understand how particles elute from the columns. The void volume is typically designated as fractions 1-5, which do not contain any detectable EV types. Results for the elution of particles from the first-dimension column comprising of Sepharose CL-2B (FIG. 13D) corresponds to the originally published results by Boing et al.³³, where the largest amounts of EVs elute in fractions 6-9 (the first four EV-containing fractions). Validation of negative controls, dPBS, and void volumes, are shown with representative TEM images (FIG. 17A). Void volume concentrations and particle sizes of 1D- and 2D-SEC are shown in FIG. 17B. Other particles, such as HDL and LDL, elute in later fractions, corresponding to the increased concentration particles in fractions 10-15 (FIG. 17C), as observed by Boing and colleagues. 33 Next, the 1D-SEC EV isolate was then loaded onto the second-dimension column and 200 μL fractions were collected and analyzed in the same manner as for the first column (FIG. 13E). Of note, particles do not start to elute from the bilayer column until fraction 10, with the chromatographic peak of particles being noticeably wider and ununiform in EV counts across the peak compared to the first column. Expecting the partial separation of EV subpopulations within this broad peak based on their hydrodynamic volume, four adjacent 200 μL fractions were pooled together to generate fractions with EV subpopulation-enriched samples. Each set of four fractions, i.e., 10-13, 14-17, and 18-21, were pooled together based on their elution order to create the following samples: “2D-Early”, “2D-Middle”, and “2D-Late”, respectively.

### Characterization of Intact EVs in Pooled Fractions Following One- and Two-Dimensional Separation

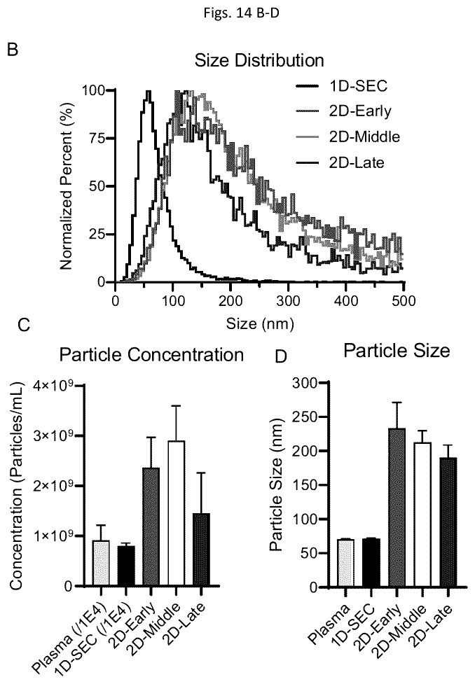

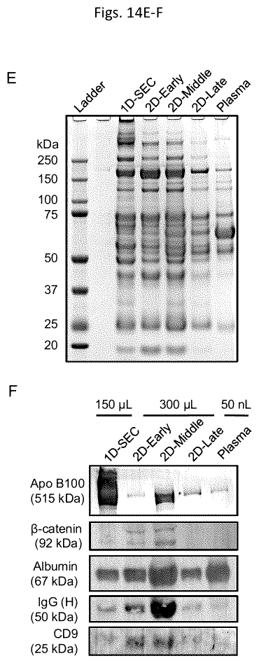

Qualitative analysis of intact EVs was performed by TEM imaging (FIG. 14A). EVs were detected in 1D-SEC as well as all fractions from 2D-SEC. The presence of EVs was visually confirmed by observing that the isolated particles have the distinctive classically cup-shaped EV feature. Visually, the imaged EVs are similar in morphology; however, slightly larger particles were observed in the 2D-SEC fractions compared to 1D-SEC.

Quantitative EV and NVEP particle size and concentrations in plasma and the fractions that were collected from 1D-SEC and 2D-SEC isolates were analyzed by NTA (FIGS. 14B-14D), N=3. Note that the NTA technology does not differentiate EVs, NVEPs, and other particles of various origin and reports the measurements for all types of EVPs and lipoprotein particles together as for generic “particles”. First, the size distributions of the isolated particles were evaluated for each fraction (FIG. 14B). 1D-SEC showed a maximum size distribution at 57 nm, while the 2D-SEC maximal range between 107-132 nm, corresponding to a size distribution shift between 50-75 nm from 1D-SEC to 2D-SEC. Furthermore, all 2D-SEC fractions show enrichment of larger extracellular particles >100 nm compared to 1D-SEC (e.g., a larger percentage of each 2D-SEC fraction is comprised of larger particles compared to 1D-SEC). Enrichment of larger extracellular vesicles and particles becomes evident by assessing the mean particle size (FIG. 14C). Plasma showed a mean particle size of 70±2 nm and the 1D-SEC EV isolate of 71±1 nm. The 2D-Early, 2D-Middle, and 2D-Late EV fractions demonstrated a mean particle size of 233±38, 212±17, and 190±19 nm, respectively. The similarity in particle size between plasma and 1D-SEC is expected due to the high particle recovery rate and significant overlap between heterogenous EV populations in both samples. The detected increase in the mean particle size in the 2D-SEC fractions indicates a high degree of depletion of smaller particles while also enriching for larger EVs, therefore increasing the total mean particle size.

Particle concentration was assessed (FIG. 14C). On average, plasma was measured to have a concentration of ˜9.1E12±3.1E12 particles/mL. The average concentration of 1D-SEC was measured to be ˜8.0E12±6.4E11 particles/mL, yielding ˜88% recovery of particles when isolating by SEC, which is consistent with previous studies^{33,56}. The measured particle concentrations of the 2D-Early, 2D-Middle, and 2D-Late fractions were ˜2.4E9±6.1E8, 2.9E9±7.0E8, and 1.5E9±8.1E8 particles/mL, respectively. These values correspond to ˜0.03%, 0.03%, and 0.02% particle recovery compared to the starting plasma. The lower concentration and recovery rates are expected since particles are subjected to a second column where further sample cleanup and separation take place (explained below in the sections describing western blot and proteomic data). Note that in FIG. 14C, the plasma and 1D-SEC concentrations are divided by 1E4 to clearly show the results for the 2D-SEC fractions.

### Protein Characterization in Pooled EV Fractions Following One- and Two-Dimensional SEC Separation

To assess the protein content in each EV fraction, SDS-PAGE was performed (FIG. 14E). There are several key differences between the samples. The 1D-SEC EV isolate contains more high molecular mass proteins that are above the top band of the ladder (>250 kDa); one of these proteins is presumably apolipoprotein B100 (>500 kDa) and confirmed by western blotting (FIG. 14F). Another key feature can be observed in the predominant band in the plasma lane (between 50-75 kDa), presumably corresponding to albumin. This band is expectedly the largest in the plasma lane; however, it is substantially less prominent in all EV fractions, indicating substantial albumin depletion from SEC isolation.

To confirm the hypothesis that free plasma proteins are significantly depleted and that EV proteins are enriched within the EV fractions, western blotting assays were performed on several proteins of interest (FIG. 14F). Apolipoprotein B100, albumin, and IgG heavy chain (H) were blotted for free plasma proteins and β-catenin and CD9 were blotted as characteristic EV-related proteins. Overwhelmingly, apolipoprotein B100 is the largest band in the 1D-SEC EV isolate, indicating high abundances of LDL particles that are isolated by this method. Meanwhile, in the 2D fractions, the apolipoprotein B100 band decreases dramatically, illustrating the efficient removal of LDL particles by the use of the second-dimension column. The 2D-Middle fraction contains the most prominent bands, correlating to the chromatographic apex from the 2D-SEC column. One hundred fifty μL PPP equivalent was used for 1D-SEC, and 50 nL of plasma was used as to not overload the gel lanes and 300 μL PPP equivalent was used for 2D-SEC in order to obtain enough signal.

### Proteomic Profiling of Plasma-Derived EVs Isolated by 1D- and 2D-SEC

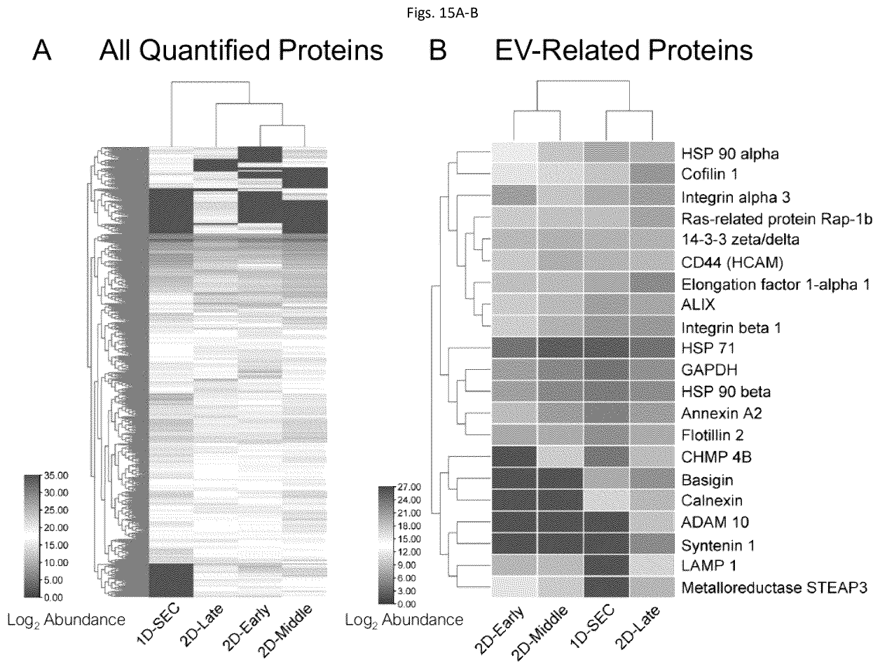

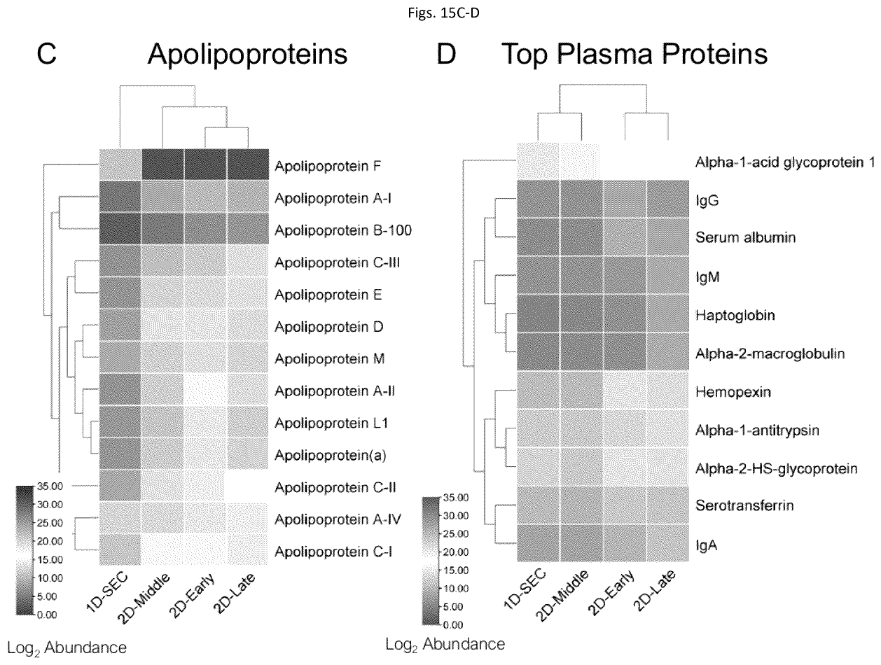

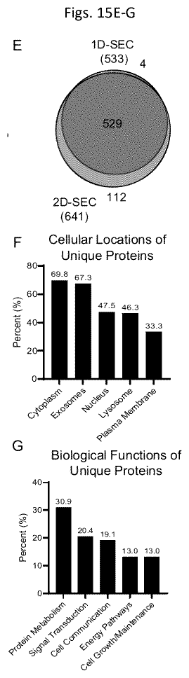

NanoLC-MS/MS-based quantitative proteomic profiling was performed to determine the protein abundances of plasma-derived EV isolates generated by 1D- and 2D-SEC. The relative abundance of EV-related proteins^{13,22}, apolipoproteins, and the most predominant free plasma proteins⁵⁷ were compared to determine the level of EV enrichment and protein depletion. Unsupervised hierarchical clustering of the experimentally determined protein abundance levels in all examined samples reveals several key differences in EV isolates generated by 1D-SEC compared to 2D-SEC (FIG. 15A). Due to the differences in protein identifications and determined abundance levels, EV isolates enriched by 1D-SEC cluster further away from the 2D-SEC EV fractions. Notably, two groups of proteins were not detected using 1D-SEC alone but were readily identified in one or more of the 2D-SEC fractions, as discussed in further detail below.

To investigate the proteins that are reportedly related to EVs based on the literature in more detail, the determined abundance values for 20 representative EV-related proteins, along with a negative EV marker (calnexin), were extracted and evaluated using unsupervised hierarchical clustering (FIG. 15B). Overall, 14 out of the 20 extracted proteins were identified and quantified in both 1D-SEC and all 2D-SEC fractions (e.g., 14-3-3 zeta/delta, ALIX, annexin a2, flotillin 2, etc.), confirming both 1D-SEC and 2D-SEC are efficient in recovering EV-related proteins at slightly different abundance levels. Four EV-related proteins out of the selected 20 EV-characteristic proteins were only identified in 2D-SEC fractions: disintegrin and metalloproteinase domain-containing protein 10 (ADAM10), lysosome-associated membrane glycoprotein 1 (LAMP1), syntenin 1, and metalloreductase STEAP3 (TSAP6), indicating that the use of fractionation can increase the depth of protein coverage in plasma-derived EVs, due to the decreased dynamic range of the isolates and the diminished extent of contamination with apolipoproteins and free plasma proteins.

Lipoprotein particles are a major component in plasma, which can be partially depleted using a Sepharose CL-2B column. However, this approach does not result in sufficient depletion in order to detect low abundant proteins in EVs, where coelution with EVs during sample processing can lead to significant contamination and downstream hindrance of the detection of EV-related proteins. To evaluate the level of contamination of EV isolates with lipoprotein particles, the experimentally determined abundance values for all identified apolipoproteins were investigated using unsupervised hierarchical clustering (FIG. 15C). All apolipoproteins are higher in abundance in 1D-SEC isolates and lower in abundance in all 2D-SEC fractions, emphasizing that the use of a second-dimension column acts as another cleanup as well as fractionation step for complex samples. Quantitative profiles for key apolipoproteins, AI and B100, were investigated. Apolipoprotein AI, the main protein in HDL with a diameter of ˜8 nm^{28,58}, exhibits ˜62-fold, ˜24-fold, and ˜40-fold lower abundances in the 2D-Early, Middle, and Late fractions, respectively, compared to the 1D-SEC technique. Similarly, apolipoprotein B100, the main protein in LDL with diameters of ˜22-28 nm²⁸, exhibits ˜19-fold, 6-fold, and 30-fold lower abundances in the 2D-Early, Middle, and Late fractions, respectively, compared to the 1D-SEC EV isolate. These findings support the theoretical mechanism of SEC, where larger particles elute first, followed by smaller particles, which spent more time in the pore network of the SEC stationary phase.

Top abundance free plasma proteins were also investigated for their quantitative levels among the various collected fractions (FIG. 15D). Intriguingly, a trend can be observed where all Early and Late fractions have lower abundance levels of the most concentrated free plasma proteins that are in plasma compared to 1D-SEC, causing the fractions to cluster together. However, for several top plasma proteins (serum albumin, IgG, and IgM) the Middle fraction demonstrated levels of abundance similar to 1D-SEC. As observed by NTA, the Middle fraction contains the highest concentration of vesicles and particles (FIG. 14C). Recently, there has been growing evidence that free plasma proteins interact with EVs, creating a corona, which may be a key feature in influencing cellular recognition and uptake, making it nearly impossible to deplete all highly abundant free plasma proteins and protein complexes during sample processing and EV enrichment^{59,60}. This could be an indication of the corona-based binding effect being more pronounced in the Middle fraction due to the increased particle concentration compared to the adjacent 2D-SEC fractions. However, further investigation into the EV corona, its composition, and its influence on the surrounding environment needs to be conducted.

Within the heat map shown in FIG. 15A, there is a group of 112 proteins that are not identified in 1D-SEC, but are uniquely identified in subsequent 2D-SEC fractions, driving 1D-SEC to cluster away from 2D-SEC fractions based on the corresponding quantitative proteomic profiles, further visualized by a Venn diagram (FIG. 15E). The proteins within the unique 2D-SEC group are either EV-related proteins, mentioned above, or EV-related protein family members (e.g., ADAM10, LAMP1, syntenin 1, TSAP6, alpha-catenin, elongation factor 1 beta, etc.). There are four proteins unique to 1D-SEC: apolipoprotein F, complement component C8 alpha, C-reactive protein, and prolactin-inducible protein (PIP; which has been found to play a role in breast cancer proliferation and resistance to treatment with tamoxifen⁶¹). The remaining 529 identified proteins are shared between 1D- and 2D-SEC, which include EV-related proteins, apolipoproteins, and free plasma proteins and complexes. Gene Ontology (GO) term enrichment analysis illustrates the cellular locations (FIG. 15F) and biological functions (FIG. 15G) of the 112 proteins that are unique to the 2D-SEC fractions. The top five cellular locations are areas where EV-related proteins are expected to be found (cytoplasm, nucleus, plasma membrane, etc.) and the top five biological functions are associated with EV-related proteins (e.g., signal transduction, cell communication, cell growth).

To validate the extent of EV fractionation and free plasma protein depletion in the 2D-SEC approach, a set of 2D fractions was recombined and pooled together (named Remix) and analyzed by nLC-DIA-MS. By extracting apolipoproteins and clustering them together with 1D- and 2D-SEC, the Remix sample clusters furthest away from other samples, indicating the Remix sample has the least similarities among samples (FIG. 19A). There are seven apolipoproteins (F, M, AII, (a), CII, AIV, and CI) that were not detected in the Remix sample, presumably due to the extra sample processing steps taken, as these apolipoproteins were the lowest abundance among the 2D-SEC fractions. Notably, in the Remix sample, apolipoprotein AI and B100, are both lower in abundance compared to the 1D-SEC sample and less than or equal to the 2D fractions. This observation is exemplified by the corresponding bar charts in FIGS. 19B-19D. Note that in FIGS. 19C and 19D, 1D-SEC abundances are divided by 10 to clearly show the results for the 2D-SEC and Remix fractions.

Characterization of Plasma-Derived Intact EVs from Prostate Cancer Donors and Controls by TEM

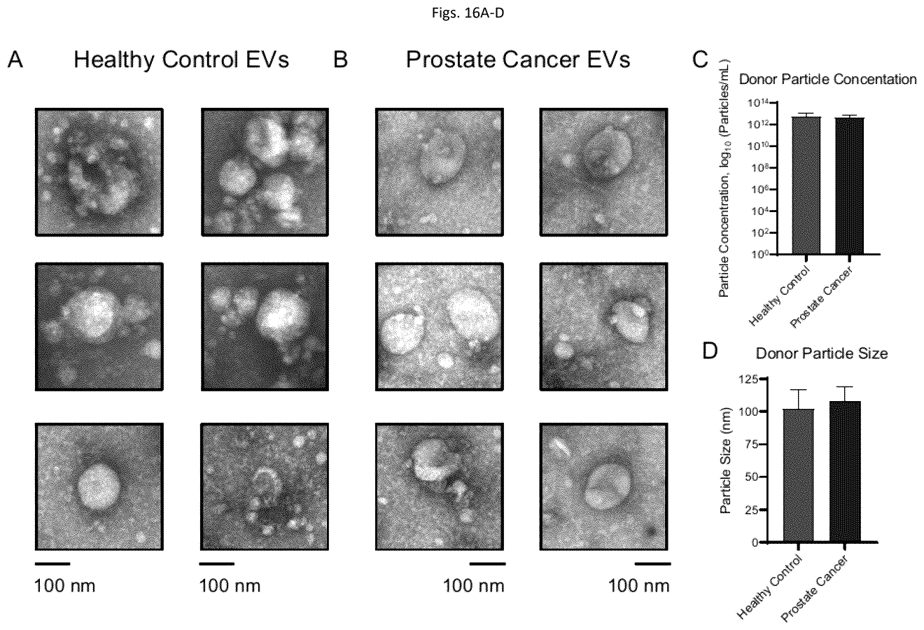

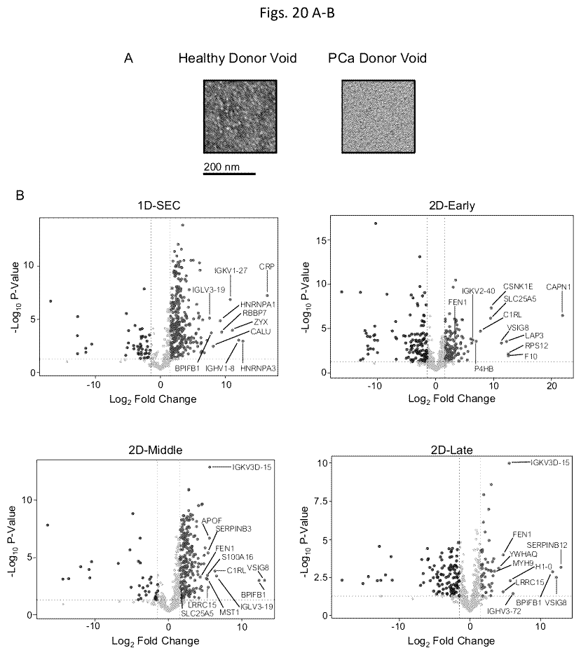

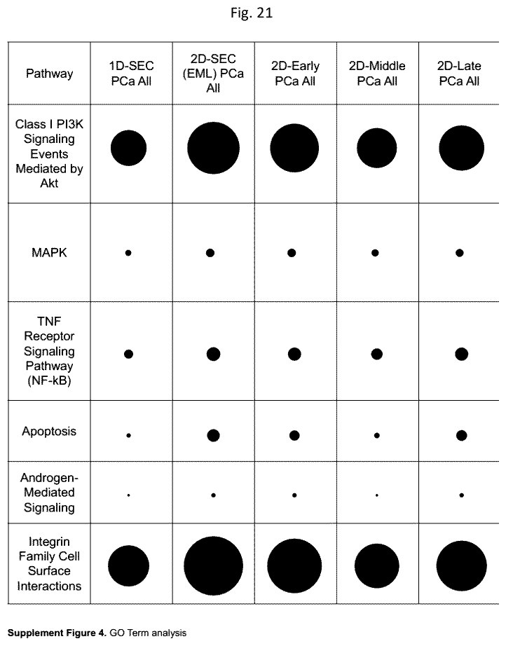

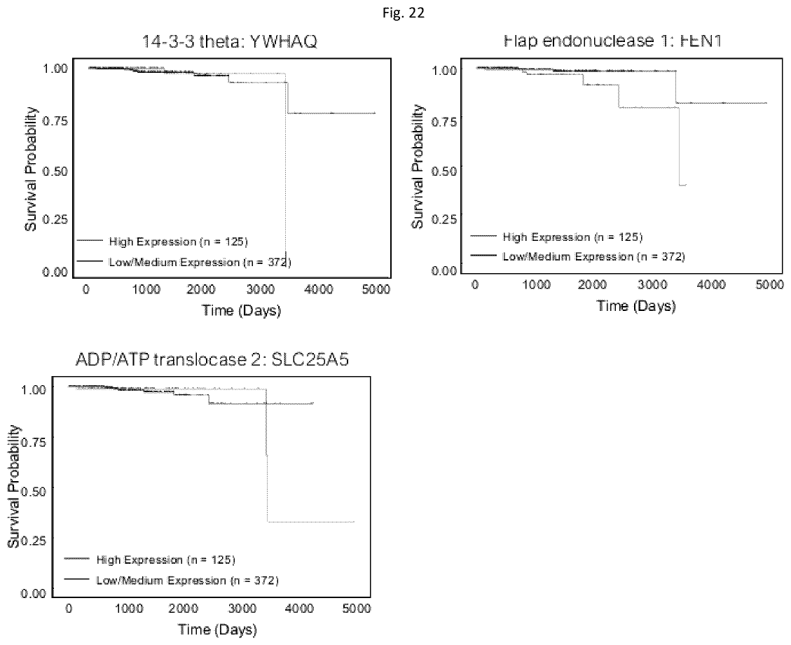

To confirm the presence of EVs from self-declared healthy donors and PCa donors, TEM imagining was performed on 1D-SEC EVs (FIGS. 16A and 16B, respectively). For both healthy control donors and PCa donors, six representative images of isolated particles are shown and are within the expected size range of ˜20-200 nm. The larger particles show the characteristic EV feature of cupping. Representative TEM images of SEC void volume samples from healthy and PCa donors are shown in FIG. 20A.

Characterization of Plasma-Derived Intact EVs from Prostate Cancer Donors and Controls by NTA

The same healthy control and PCa donor samples that were used for TEM imagining were subjected to NTA to measure particle concentration and size (FIGS. 16C and 16D). Ten healthy control donors had a particle concentration ranging between 5.9E11-2.0E13 particles/mL and ten PCa donors had a particle concentration ranging between 1.1E12-1.0E13 particles/mL. While many factors can alter EV biogenesis and concentrations in vivo, including hydration, diet, exercise, circadian cycles, stage of the disease, etc.⁶², plasma-derived EVs are a mixture of EVs generated from nearly all cells and tissues of the body, resulting in total EV concentrations that can vary, but overall are similar (i.e., on the same order of magnitude). On average, healthy control donors had a particle size of 102±15 nm and PCa donors had a particle size of 108±11 nm (FIG. 16D). The similarity in particle size indicates the similarity and consistency in the isolation method performance and the overall plasma EV size ranges in both types of donors.

Proteomic Profiling of Plasma-Derived EVs from Prostate Cancer and Control Donors Isolated by 1D- and 2D-SEC

Investigation of the plasma-derived EV proteome from healthy and PCa donors isolated by 1D-SEC as well as 2D-SEC was performed by nLC-DIA-MS (FIGS. 18A-18D). Unsupervised hierarchical clustering between all EV fractions reveals that all healthy control-derived samples cluster together and all PCa donor samples cluster separately. This division is an indication of the similarities and differences among protein identifications and abundances in the samples derived from each donor type (FIG. 18A).

Protein identifications reveal that the majority of detected proteins in EV isolates are shared between healthy and disease sample types (FIG. 18B). As expected, shared proteins include free plasma proteins such as albumin and immunoglobins as well as EV-related proteins such as CD9 and annexin A2. Notably, prostatic acid phosphatase (PAP) was identified in the 2D-Early and 2D-Late disease fractions. Due to the absence of PAP in the 1D-SEC fraction, identifying this protein in the 2D-Early and 2D-Late fractions is an indication that depletion of high abundance free plasma proteins and subsequent fractionation of EVs can yield in deeper proteome coverage, identifying proteins associated with PCa that could otherwise be not detected.

Principal component analysis (PCA) was performed on all fractions, with principal components 1 and 2 showing a clear separation between healthy donors and PCa donors (FIG. 18C). Each biological replicate is marked by a circle and the 95% confidence intervals are marked by the ovals on each plot. Driven by the qualitative protein identifications and quantitative profiling, there is no intersection between the confidence intervals determined for healthy donors and PCa donors in 1D-SEC isolation. In the 2D fractions, there is a slight overlap of the confidence intervals; however, all biological donors mostly cluster together. Notably, healthy control samples cluster tightly together in the 2D-Middle fraction, possibly indicating that because the middle fraction is the apex of the chromatographic peak, less variation could be observed between samples. Volcano plots comparing PCa and healthy donors of each fraction illustrate up- and down-regulated proteins (FIG. 18D). The top 10 differentially expressed proteins (DEPs) and other selected proteins of interest are labeled in FIG. 20B.

### Assessment of 2D-SEC Performance for Plasma-Derived EV Enrichment and Fractionation

A 2D-SEC method for in vivo plasma-derived EV enrichment and fractionation was developed and tested on plasma from healthy controls and PCa donors. Two-dimensional separation has previously been shown to decrease background matrix contamination in order to increase the depth of molecular profiling of analytes of interest. Plasma is a minimally invasive sample type that is rich in biological information, rendering it appealing for diagnostic and therapeutic use. However, the vast majority of protein mass within plasma is dominated by highly abundant free proteins (i.e., serum albumin, immunoglobins) and lipoprotein particles (HDL, LDL, VLDL, etc.), which can hinder the detection of lower abundance proteins of interest. The inventors demonstrate that the 2D-SEC method is effective, practical, and well-suited for further purification, enrichment, and fractionation of plasma-derived EVs to enable diagnostic protein marker discovery.

One-dimensional SEC for isolation of total plasma-derived EVs can be suitable for certain applications, as demonstrated by Boing and coworkers³³. However, this technique may not be sufficiently efficient for the recovery and enrichment of lower abundance EV subpopulations and the corresponding molecular species when analyzing highly heterogeneous EVs from complex biological matrices. Blood circulates EVs of various sizes that originate from different cell types and tissues in the body (e.g., liver, heart, pancreas, white blood cells, red blood cells) rendering plasma to be an extremely heterogeneous mixture of EV subpopulations. One-dimensional SEC separation has been shown to enrich EVs along with other particles from plasma; however, the inventors hypothesized that an additional dimension of separation would be beneficial to enriching and unmasking EV subpopulations.

Sepharose CL-2B, as an SEC stationary phase with an exclusion limit of 75 nm, has become frequently used in studies that require EV enrichment, which has demonstrated its ability to isolate particles >70 nm in diameter³³. During the SEC process, larger particles that do not fit inside the pores of the stationary phase beads elute first and smaller particles elute later, as the smaller particles take a longer path through the column. Taking advantage of the property that larger extracellular particles elute from an SEC column first, enrichment for EV subpopulations can be performed in the several 2D fractions, where expectedly larger particles are collected in the earlier fractions followed by smaller particles in the later fractions. Using a bilayer column in the second SEC dimension, where the two stationary phases used have different fractionation ranges enables EV separation with an extended total fractionation range of the multilayer column. Sephacryl 1000 has an exclusion limit of ˜400 nm, allowing any particle that is <400 nm (encompassing the vast majority of EV subpopulations) to effectively interact with the pore network of the stationary phase. This interaction enables separation of EV subpopulations, which cannot be achieved using only a Sepharose CL-2B or similar column. Once analytes have passed through the Sephacryl 1000 layer, they enter the Sephacryl 500 layer and interact with this SEC stationary phase, which has an exclusion limit of ˜42 nm. This layer will allow particles that are <42 nm to enter the pores and separate further. Notably, HDL and LDL are ˜8 nm and ˜22-28 nm in diameter, respectively, and the majority of EVs are >30 nm (e.g., exosomes, microvesicles, supermeres, exomeres)¹³; this layer allows EVs to further separate away from HDL and LDL particles. The combined use of Sephacryl 1000 and 500 allows the effective fractionation range of analytes to increase compared to CL-2B (or similar a resin) alone, while resulting in further depletion of lipoprotein particles.

The distributions of particle size and concentration determined by NTA support the expected EV fractionation trends using the biphasic column (FIGS. 14B-14D). The starting material, plasma, and 1D-SEC isolates generated using Sepharose CL-2B have similar average particle concentration and size distributions, indicating that the depletion of lipoprotein particles and large protein complexes is not as efficient. However, following the biphasic column packed with Sephacryl resins, the particle concentration decreases by ˜1E4 particles/mL overall, presumably due to the depletion of lipoprotein particles, which is supported by western blotting and proteomic results (FIG. 14F and FIG. 15C). Even at the lower particle concentration, the apex of the EV chromatographic peak can still be observed in the results acquired using NTA, 1D-PAGE, and western blots, as the 2D-Middle fraction has the highest concentration of particles out of the fractions. The overall decrease in particle concentration is accompanied by the increase in particle size in the 2D fractions, indicating that the majority of the depleted particles were smaller in size. Therefore, the overall distribution of particle sizes increased in 2D-SEC, with all 2D fractions exhibiting larger mean particle sizes than the 1D-SEC isolate. A trend is also observed in particle size where the 2D-Early fraction has the largest mean particle size and decreases with 2D-Middle and 2D-Late, confirming that particle size decreases in the order of elution from the SEC column (FIG. 14D). This is an indication that each 2D fraction is enriched with different EV subpopulations (i.e., 2D-Early is enriched with larger EVs and 2D-Late is enriched with smaller sized EVs). To further explore how the depletion of highly abundant particles affected the enrichment of EVs and EV subpopulations, the inventors employed quantitative proteomic profiling.

Proteomic data demonstrate numerous identified and quantified proteins in the 2D-SEC generated isolates, many of which are reported as EV-related proteins, even though the particle concentration decreases dramatically in the 2D fractions in comparison to 1D SEC isolates (FIGS. 15A and 15B). The evaluation of the detected EV-related proteins and their abundances confirms that various EV subpopulations are partially separated on the second column. Multiple EV-related proteins have similar abundances across fractions (e.g., 14-3-3 zeta/delta has a log 2 abundance of 19.3, 18.9, 19.4, and 19.5 in 1D-SEC, 2D-Early, 2D-Middle, and 2D-Late, respectively). However, some EV-related proteins demonstrate more substantial abundance differences across 2D-SEC fractions, including syntenin 1 (which was reportedly characteristic of NVEPs⁶³). Syntenin 1 was only identified in the 2D-Late fraction, indicating that some fractions could either contain different concentrations of the same EV subpopulations or that different EV subpopulations may contain various amounts of specific proteins, leading to the detection of diverse EV-related protein quantitative profiles. By far, the highest abundance protein apolipoprotein B100 (associated with LDL) was observed with log 2 abundance levels of 30.7, 26.7, 28.2, 25.8 in 1D-SEC, 2D-Early, 2D-Middle, and 2D-Late, respectively (FIG. 15C). As expected, the highest overall relative abundance of apolipoprotein B100 is found in 1D-SEC and significantly decreases when using the second SEC dimension. This is exemplified in the unsupervised hierarchical clustering of the acquired quantitative proteomic profiles of apolipoproteins, where the 1D-SEC quantitative profiles clustered further away from all 2D fractions (FIG. 3C). Furthermore, all remaining detected apolipoproteins (F, AI, CIII, E, D, M, etc.) have their respective highest abundances in 1D-SEC and decreased abundance levels in all 2D fractions. For example, apolipoprotein E (Apo E), which is an important protein for the regulation of lipid and lipoprotein homeostasis that is cleared by the liver, is dramatically decreased in all 2D fractions compared to 1D-SEC. It is advantageous to deplete apo E from patient plasma-derived EV isolates for the development of EV-based therapeutics to avoid degradation by the liver⁶⁴. Notably, apolipoprotein F was not detected in any of the 2D fractions, but is unique to 1D-SEC. Apo F is one of the four unique proteins to 1D-SEC in the Venn diagram (FIG. 15E). Apo F plays a role in modulating cholesteryl ester transfer protein and binds primarily to HDL and some to LDL. Presumably, the depletion of HDL in the 2D fractions leads to this loss of apolipoprotein F65. Apolipoprotein AII is another constituent of HDL with log 2 abundances of 26.0, 17.1, 20.7, 19.6 in 1D-SEC, 2D-Early, 2D-Middle, and 2D-Late, respectively, indicating depletion of HDL along with apolipoprotein AI, E, and F. Strikingly, similar trends were not observed for classic highest abundance free plasma proteins, including human serum albumin (FIG. 15D). Generally, the 1D-SEC and 2D-Middle fractions were found to have similar abundances of top free plasma proteins and the 2D-Early and 2D-Late had lower abundances of free plasma proteins. For example, serum albumin exhibited the trend of having larger abundances in 1D-SEC and 2D-Middle with lower abundances in 2D-Early and 2D-Late. In this regard, 1D-SEC depletes free plasma proteins, and the addition of a second column does not enhance much further depletion of serum albumin, indicating that serum albumin co-elutes with EVs via EV corona interactions. Although, other highly abundant plasma proteins (e.g., alpha-1-acid glycoprotein 1, alpha-2-HS-glycoprotein, hemopexin) were found to have lower abundances in the 2D fractions, demonstrating some depletion of free plasma proteins.

Completely depleting highly abundant endogenous proteins in EV isolates from biofluids-considered to be contaminants co-isolated with EVs from complex biological matrices, such as plasma—is difficult using the current EV isolation methods. There has been evidence that supports the notion that many free plasma proteins non-covalently interact with EVs and form several protein layers around EVs, known as the protein corona, including serum albumin, apolipoproteins, immunoglobins, and complement proteins^{59,66,67}. Further investigations have illustrated that the top abundance free plasma proteins that are associated with the EV protein corona contribute to specific cell targeting and modulate endocytosis of EVs into cells^{68,69}. Therefore, it is important to note that while plasma protein depletion methods have been developed, isolation of EVs may also result in co-isolation of plasma proteins incorporated as integral parts of the EVs or as constituents of the EV corona system. This notion could explain why the present proteomic results show similar levels of top abundance plasma proteins across 1D- and 2D-SEC. Thus, the detection of top abundance plasma proteins in EV isolates may mostly correspond to the EV protein corona combined with plasma proteins incorporated into EVs. Additionally, 2D-SEC is more effective at removing free lipoprotein particles rather than free plasma proteins. To test this, the 2D fractions were recombined together in a Remix sample (FIGS. 19A-19D). All detected apolipoproteins in the Remix sample were lower in abundance levels than in the 1D-SEC EV isolates and less than or equal to in abundance levels compared to the 2D-SEC fractions. This is an indication that the depletion of lipoprotein particles is enhanced by the use of the second column. If depletion was not efficient or significant, the apolipoprotein abundances in the Remix sample should expectedly be approximately equal to 1D-SEC.

Evaluation of 2D-SEC in EV Isolation and Fractionation Using Plasma from Prostate Cancer and Control Donors

An objective of the present technology was to decrease the dynamic range of EV isolates mainly by depleting the level of co-isolation of lipoprotein particles and free plasma proteins while also enabling the fractionation of EVs by size. However, the complete removal of all highly abundant plasma proteins and lipoprotein particles from plasma-derived EV isolates is likely impossible with the current methods because these common contaminants might be bound to EVs as constituents of the EV protein corona system. By narrowing the dynamic range of the major plasma constituents and EV concentration levels in the collected EV isolates, the inventors aimed to improve the MS-based proteomic profiling sensitivity in the detection of low abundance EV proteins that are involved in pathways that promote tumorigenesis and cell proliferation, including the PI3K/AKT/mTOR (PAM), mitogen-activated protein kinase (MAPK), and the nuclear factor kappa-light-chain-enhancer of activated B cells (NF-κB) pathways^{70,71}. The PAM pathway has been reported to be the most activated pathway that leads to cell proliferation in cancer while also having the potential to lead to treatment resistance. The inventors have applied our 2D-SEC isolation method to plasma from PCa donors, where the inventors detected and quantified several key proteins reported as oncogenic, prognostic markers, or tumor suppressors in the 2D-SEC fractions.

For example, gene ontology (GO) term enrichment analysis of the proteomic profiling data acquired for plasma EVs isolated by 1D-SEC from PCa donors identified 60 proteins that are associated with Class I PI3K signaling events (FIG. 21). However, the inventors were able to increase the number of identified proteins by 47%, corresponding to a total of 88 proteins that are associated with PI3K signaling in the 2D-SEC fractions. Unique proteins identified in the 2D-SEC fractions include eukaryotic translation initiation factor 2 subunit 1 and 4A-I (EIF2S1/4A1), nucleoside diphosphate kinase A (NME1), and heterogeneous nuclear ribonucleoprotein A1 (HNRNPA1). Eukaryotic initiation factors (eIFs) are instrumental in the translation of mRNA, with dysregulation of mRNA being a key feature in tumorigenesis; therefore, eIFs are targets of interest for developing cancer therapies to help regulate mRNA expression⁷². Similarly, when HNRNPA1 is sumoylated, it can modulate the sorting and packaging of miRNAs into EVs and can trigger immune responses⁷³.

The MAPK pathway involves activation of an EGFR to start a signal cascade that goes to the nucleus to activate transcription factors to trigger mitosis and cell proliferation⁵. Nine proteins were found in 1D-SEC EV isolates from PCa patients that are associated with the MAPK pathway, while 13 proteins were identified and quantified in the 2D-SEC fractions from the same PCa donors, corresponding to a 44% increase in identifications (FIG. 21). Unique proteins include GTP-binding nuclear protein Ran (RAN), importin subunit beta-1 (KPNB1), and heat shock protein beta-1 (HSPB1), all of which have been found to be associated with various cancers. RAN, a major nucleocytoplasmic transport cofactor, can be activated through the MAPK pathway, shuttling molecules between the cytoplasm and nucleus during a positive feedback loop that promotes cell growth and tumorigenesis, mostly controlling the association of nuclear transport receptor (NTR)-cargo^{74,75}. KPNB1, a member of the importin beta protein family involved in nucleocytoplasmic transport, binds to cargo proteins in the cytoplasm intended to be transported to the nucleus. Heat shock proteins are known to stabilize key proteins in the MAPK pathway, such as Raf, Akt, and HER276. Therefore, proteins detected in circulating EVs might be informative about perturbations in the MAPK pathway in a remote growing tumor, e.g., in PCa patients.

Furthermore, the NF-κB pathway is a response to a variety of different mechanisms, such as immune response, proliferation, and cell survival, with dysregulation leading to a wide range of diseases, including cancer⁷⁷. Overactivation of the NF-κB pathway promotes cell survival and bypasses apoptosis. The inventors identified 14 proteins associated with NF-κB in 1D-SEC and 22 proteins in the 2D-SEC fractions, corresponding to a 57% increase in identifications (Figure S4). All 14 proteins identified in 1D-SEC were also identified in 2D-SEC, while eight additional unique proteins were identified in 2D-SEC fractions. Among the proteins uniquely detected in 2D SEC isolates are thioredoxin (TXN), small ribosomal subunit protein RACK 1 (GNB2L1), lamin A/C (LMNA), and lamin B1 (LMNB1). Thioredoxin has been a protein of interest for cancer studies due to its redox capabilities in many different tumor types that either undergo oxidative or hypoxic stress. Additionally, thioredoxin can reduce a cysteine residue on the NF-κB protein, which will increase its DNA binding activity, leading to transcriptional gene activation⁷⁸. RACK1 has been shown to interact with prostate tumor-overexpressed gene 1 protein (PTOV1), which promotes mRNA translation for pro-oncogenic effects such as bypassing apoptosis⁷⁹. Lamin proteins play an important role in maintaining the regulation, expression, and stability of genetic material. Overexpression of lamins can occur in certain types of prostate cancer⁸⁰. Interestingly, the unique proteins that are identified in the 2D-SEC-based approach are associated with the nucleus, nuclear transport, or DNA and RNA binding, all of which influence how proteins and nucleic acids are packaged into EVs and then excreted into the extracellular matrix. Additionally, the 2D-SEC method was able to identify 20 proteins attributed to apoptosis, whereas the 1D-SEC only identified six proteins, corresponding to a 233% increase in identifications. Similarly, 1D-SEC was able to identify three proteins associated with androgen-mediated signaling, an important signaling cascade for the prostate gland, and a target pathway for PCa chemotherapy, whereas 2D-SEC identified six proteins. While some gains in the number of protein identifications are modest, others are substantial, with 2D-SEC illustrating enrichment in all pathways of interest that lead to cell survival, proliferation, and apoptosis. The different abundance levels of specific proteins of various origin could be an indication that certain pathways can be overrepresented and others to be underrepresented within EVs, as the biological mechanisms and interactions between EV biogenesis (i.e., the ESCRT pathway or budding) and other molecular pathways of the cell metabolism in health and PCa (i.e., the PAM, MAPK, or NF-κB) remain largely unexplored. However, there is recent evidence that vacuolar protein sorting 4B (VPS4B, an important protein involved at the final stages of the ESCRT pathway) can regulate apoptosis through the MAPK pathway in osteoarthritis⁸¹. Furthermore, in other cancer studies, VPS4B has been shown that overexpression tends to lead to cancer cell proliferation and poor prognosis, without knockouts leading to tumor cell apoptosis⁸²-84 However, the involvement and interactions between the ESCRT pathway and cancer-related pathways still need to be elucidated.

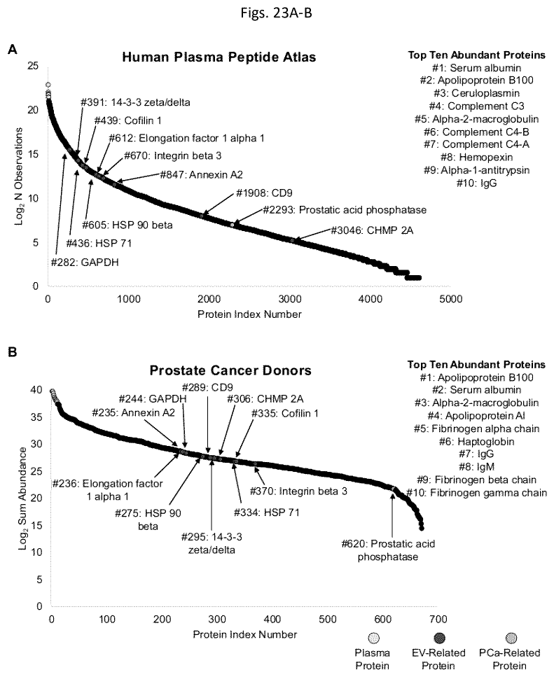

Next, to gain further insight into the enriched proteins by 2D-SEC, protein identification and abundance results were evaluated in EVs generated by 2D-SEC fractions (i.e., detected at higher abundance levels in 2D-SEC or unique to 2D-SEC). Seventy-nine proteins were found that are enriched and unique to 2D-SEC; eight of those proteins were also among the top 10 DEPs (i.e., between PCa and control samples) within 2D-SEC, including 14-3-3 theta (YWHAQ), ADP/ATP translocase 2 (SLC25A5), flap endonuclease 1 (FEN1). Additionally, heterogeneous nuclear ribonucleoprotein A2/B1 (HNRNPA2B1) was also identified as a DEP and found in both 1D-SEC and 2D-SEC. All four proteins have been found to play significant roles in promoting prostate tumor growth and metastasis⁸⁵⁻⁸⁸ (FIG. 20B). Normally, YWHAQ binds to several partners, including 3-phosphoinositide-dependent protein kinase 1 (PDK1), modulating its kinase activity⁸⁹. Moreover, YWHAQ has been found to be increased in PCa and promotes cell proliferation with other isoforms of the 14-3-3 family, also having various effects on other cancer types^{86,90}. For cellular energy metabolism, SLC25A5 mediates the import of ADP and the export of ATP into and out of the mitochondrial matrix for ATP synthesis and can be an indicator of carcinogenesis. It has been reported in PCa and other cancer types that dysregulation of SLC25A5 and its family members affects nucleotide metabolism and, eventually, neoplastic transformation⁸⁷. FEN1 is a nuclease involved in DNA replication and repair. It has been found that FEN1 inhibition promotes DNA damage in PCa cells, which is an adventurous target for cancer treatment⁸⁵. HNRNPA2B1 regulates the packaging of miRNAs into EVs and is an RNA methylation reader, specifically N6-methyladenosine (m6A) 88. It has been found that HNRNPA2B1 can promote the transport of KRAS mRNA out of the nucleus during the activation of the MAPK signaling cascade⁹¹. Next, The Cancer Genome Atlas (TCGA) was utilized through the UALCAN portal^{92,93} under the prostate adenocarcinoma (PRAD) dataset, based on the abundance of the corresponding RNA transcripts, to illustrate the Kaplan-Meyer survival plots of the four selected proteins, that are differentially expressed, found only in 2D-SEC, correlate to poor patient survival, and agree with literature to have effects on PCa tumor growth and metastasis (FIG. 23B). Survival plots under the PRAD dataset illustrate that overexpression of each of the four proteins tends to correlate to poor patient survival, with the most significant proteins FEN1 and HNRNPA2B1 (p<0.05). Interestingly, both FEN1 and HNRNPA2B1 directly interact with DNA or RNA.

Mitochondria are versatile organelles, which have their own genome (mtDMA) and locally translated proteins that are essential for cellular energy production, having the important role of producing ATP through oxidative phosphorylation and are also involved in lipid and nucleic acid metabolism^{94,95}. Mutations and dysregulation of the highly regulated mitochondrial pathways have been shown to be reprogrammed by the PAM and NF-κB pathways during tumorigenesis to support cancer cell survival and metastasis. These pathways are of interest to be targeted in the development of new therapies. The inventors found mitochondrial ATP synthase γ (ATP5F1C) to be upregulated in the 2D-Middle and 2D-Late fractions isolated from PCa donors (Figure S3B). ATP5F1C is a subunit of the ATP synthase complex, working in conjunction with the & subunit and allowing rotation of the proton pump to occur⁹⁶. Overexpression of ATP synthase has been found in prostate, breast, and ovarian cancers, as well as glioblastoma and clear cell renal cell carcinoma, which can lead to a poor prognosis⁹⁷. Additionally, mitochondrial peroxiredoxin-5 (PRDX5) was found to be increased in the 2D-Middle and 1D-SEC fractions as well. Peroxiredoxins are a family of proteins that are part of the thioredoxin redox system (where the inventors identified thioredoxin only in 2D-SEC fractions, above). Peroxiredoxins catalyze the oxidation of TXN and have been found to play pivotal roles in tumor progression^{78,98}. Specifically, PRDX5 has recently been proposed as a target for treating castration-resistant PCa, where inhibition of PRDX5 can suppress tumor growth⁹⁹.

Additionally, histone proteins are of critical importance for DNA structure, organization, maintenance, and regulation. The inventors detected 12 histone proteins in PCa donor EVs, of which histone H1.0 (H1-0) was found to be more abundant in the 2D-Late faction, and it is also among the top 10 DEPs between PCa- and control-derived EVs enriched in this particular fraction. H1-0 is one of the five major histones, sitting outside of the nucleosome, providing structure and stabilization to the nucleosome core, which is comprised of histones H2A, H2B, H3, and H4. Moreover, the inventors identified several methylation sites on histone 3.1, specifically monomethylation on H3K79 and both dimethylation and trimethylation on H3K27. Both sites have been reported as regulators of transcriptional activation 100,101. In the context of EVs, it has been shown that both DNA and RNA species carried by EVs can have profound effects on cancer progression and metastasis 102-104. Recently, it has been shown that low amounts of tumor-derived EV DNA led to a worse prognosis and can serve as a tool for metastatic risk assessment and defining future treatment¹⁰⁵. Therefore, monitoring the presence and abundance levels of DNA- and RNA-binding proteins within EVs can be advantageous to better understand EV biogenesis and explore their diagnostic potential.

EVs and their associated cargo offer a promising role for their use in diagnostic assays, exhibiting traits of an accessible sample type that might be particularly useful in the early diagnosis and monitoring of specific diseases, such as cancer. The TME is an extremely complex area surrounding tumor cells, which is intimately involved in modulating the growth and metastasis of tumors, with various pathways that are regulated to execute cell proliferation and bypass apoptosis, including the PAM, MAPK, and NF-κB pathways. EVs have the distinct feature of carrying biomolecules that act as signals and messengers between cells via secretion (exocytosis or budding) and cellular uptake (endocytosis) that can influence these pathways. Increasing the purity of plasma-derived EV and EV subpopulation isolates has proven to be challenging without losing a substantial fraction of EVs during sample processing. Currently, there are various methods available to researchers to isolate EVs that also deplete other highly abundant plasma constituents, such as UC- and 1D-SEC-based, both of which have considerable drawbacks for enabling high purity, high throughput, and high quality EV isolation workflows. It has been shown that UC-based EV isolation is time-consuming, has low throughput, and tends to cause EV aggregation⁵⁶. While 1D-SEC is a more gentle isolation method that maintains EVs in their native state, it has been shown that this method is not ideal for high purity in vivo isolation due to the size overlap of EVs and lipoprotein particles. To overcome these challenges, the inventors developed a 2D-SEC isolation method utilizing a biphasic column that maintains EVs in their native state. The developed 2D-SEC approach is scalable (i.e., to process either larger or smaller sample volumes), has higher throughput, and can efficiently deplete lipoprotein particles and enrich EV subpopulations.

In the context of this proof-of-concept pilot study, the inventors generated plasma-derived EVs from 10 healthy controls and 10 PCa donors to probe for possible correlations between controls and PCa donors. Further studies will need to be performed that include hundreds or thousands of patient samples to determine, validate, and verify which EV subpopulations and EV-related proteins that could serve as specific and sensitive biomarkers for PCa. Our goal for this study was to explore the applicability and diagnostic potential of the developed 2D-SEC technique for EV isolation and fractionation.

### References Set 2

1 (WHO), W. H. O. Global Cancer Observatory, gco.iarc.fr/en (2024).
2 Shyamala, G., Chou, Y. C., Cardiff, R. D. & Vargis, E. Effect of c-neu/ErbB2 expression levels on estrogen receptor alpha-dependent proliferation in mammary epithelial cells: implications for breast cancer biology. Cancer Res 66, 10391-10398, doi: 10.1158/0008-5472.CAN-06-0321 (2006).
3 Liu, X. et al. Purification, identification and Cryo-EM structure of prostatic acid phosphatase in human semen. Biochem Biophys Res Commun 702, 149652, doi: 10.1016/j.bbrc.2024.149652 (2024).
4 Hong, S. K. Kallikreins as biomarkers for prostate cancer. Biomed Res Int 2014, 526341, doi: 10.1155/2014/526341 (2014).
5 WEI ZHANG, H. T. L. MAPK signal pathways in the regulation of cell proliferation in mammalian cells. Cell Research 12 (2002).
6 Velonas, V. M., Woo, H. H., dos Remedios, C. G. & Assinder, S. J. Current status of biomarkers for prostate cancer. Int J Mol Sci 14, 11034-11060, doi: 10.3390/ijms140611034 (2013).
7 Lu, Y. T., Delijani, K., Mecum, A. & Goldkorn, A. Current Status of Liquid Biopsies for the Detection and Management of Prostate Cancer. Cancer Manag Res 11, 5271-5291, doi: 10.2147/CMAR.S170380 (2019).
8 Weinstein, J. N. et al. The Cancer Genome Atlas Pan-Cancer analysis project. Nat Genet 45, 1113-1120, doi: 10.1038/ng.2764 (2013).
9 Larsson Omberg, K. E., Yuan Yuan, Cyriac Kandoth, Chris Wong, Michael R Kellen, Stephen H Friend, Josh Stuart, Han Liang, and Adam A Margolin. Enabling transparent and collaborative computational analysis of 12 tumor types within The Cancer Genome Atlas. Nat Genet 45, 1121-1126 (2013).
10 Muniyan, S. et al. Human prostatic acid phosphatase: structure, function and regulation. Int J Mol Sci 14, 10438-10464, doi: 10.3390/ijms140510438 (2013).
11 Wang, W., de Nier, C. R., Wuhrer, M. & Lageveen-Kammeijer, G. S. M. In-Depth Glycoproteomic Assay of Urinary Prostatic Acid Phosphatase. ACS Meas Sci Au 4, 117-126, doi: 10.1021/acsmeasuresciau.3c00055 (2023).
12 Anderson, N. M. & Simon, M. C. The tumor microenvironment. Curr Biol 30, R921-R925, doi: 10.1016/j.cub.2020.06.081 (2020).
13 Welsh, J. A. et al. Minimal Information for Studies of Extracellular Vesicles (MISEV2023): From Basic to Advanced Approaches. J Extracell Vesicles 13, 84, doi: 10.1002/jev2.12404 (2024).
14 Clifford Harding, J. H., and Philip Stahl. Receptor-mediated Endocytosis of Transferrin and Recycling of the Transferrin Receptor in Rat Reticulocytes. Journal of Cell Biology 97, 329-339 (1983).
15 Pan, B.-T. J., Rose M. Fate of the Transferrin Receptor during Maturation of Sheep Reticulocytes In Vitro: Selective Externalization of the Receptor. Cell 33, 967-977 (1983).
16 Shah, R., Patel, T. & Freedman, J. E. Circulating Extracellular Vesicles in Human Disease. N Engl J Med 379, 958-966, doi: 10.1056/NEJMra1704286 (2018).
17 Urabe, F. et al. Extracellular Vesicles as Biomarkers and Therapeutic Targets for Cancer. Am J Physiol Cell Physiol 318, C29-C39, doi: 10.1152/ajpcell.00280.2019 (2020).
18 Fais, S. et al. Evidence-Based Clinical Use of Nanoscale Extracellular Vesicles in Nanomedicine. ACS Nano 10, 3886-3899, doi: 10.1021/acsnano.5b08015 (2016).
19 Maas, S. L. N., Breakefield, X. O. & Weaver, A. M. Extracellular Vesicles: Unique Intercellular Delivery Vehicles. Trends Cell Biol 27, 172-188, doi: 10.1016/j.tcb.2016.11.003 (2017).
20 Mathieu, M., Martin-Jaular, L., Lavieu, G. & Thery, C. Specificities of secretion and uptake of exosomes and other extracellular vesicles for cell-to-cell communication. Nat Cell Biol 21, 9-17, doi: 10.1038/s41556-018-0250-9 (2019).
21 Harding C., H. J., Stahl P. Endocytosis and Intracellular Processing of Transferrin and Colloidal Gold-Transferrin in Rat Reticulocytes: Demonstration of a Pathway for Receptor Shedding. European Journal of Cell Biology 35, 256-263 (1984).
22 Zhang, H. et al. Identification of Distinct Nanoparticles and Subsets of Extracellular Vesicles by Asymmetric Flow Field-Flow Fractionation. Nat Cell Biol 20, 332-343, doi: 10.1038/s41556-018-0040-4 (2018).
23 Zhang, Q. et al. Supermeres are Functional Extracellular Nanoparticles Replete with Disease Biomarkers and Therapeutic Targets. Nat Cell Biol 23, 1240-1254, doi: 10.1038/s41556-021-00805-8 (2021).
24 Hoshino, A. et al. Extracellular Vesicle and Particle Biomarkers Define Multiple Human Cancers. Cell 182, 1044-1061 e1018, doi: 10.1016/j.cell.2020.07.009 (2020).
25 Anderson, N. L. & Anderson, N. G. The Human Plasma Proteome: History, Character, and Diagnostic Prospects. Mol Cell Proteomics 1, 845-867, doi: 10.1074/mcp.r200007-mcp200 (2002).
26 Gonzalez-Quintela, A. et al. Serum levels of immunoglobulins (IgG, IgA, IgM) in a general adult population and their relationship with alcohol consumption, smoking and common metabolic abnormalities. Clin Exp Immunol 151, 42-50, doi: 10.1111/j. 1365-2249.2007.03545.x (2008).
27 Gandham, S. et al. Technologies and Standardization in Research on Extracellular Vesicles. Trends in Biotechnology, doi: 10.1016/j.tibtech.2020.05.012 (2020).
28 Kreimer, S. et al. Mass-spectrometry-based molecular characterization of extracellular vesicles: lipidomics and proteomics. J Proteome Res 14, 2367-2384, doi: 10.1021/pr501279t (2015).
29 Sodar, B. W. et al. Low-Density Lipoprotein Mimics Blood Plasma-Derived Exosomes and Microvesicles During Isolation and Detection. Sci Rep 6, 24316, doi: 10.1038/srep24316 (2016).
30 Mahley, R. W., Innerarity, T. L., Rall, S. C. & Weisgraber, K. H. Plasma Lipoproteins: Apolipoprotein Structure and Function. Journal of Lipid Research 25, 1277-1294, doi: 10.1016/s0022-2275 (20) 34443-6 (1984).
31 Elena V. Orlova, M. B. S., Wah Chiu, Hiro Mowri, Louis C. Smith, Antonio M. Gotto Jr. Three-Dimensional Structure of Low Density Lipoproteins by Electron Cryomicroscopy. Biochemistry 96 (1999).
32 Ferreira, P. M. et al. Mode of induction of platelet-derived extracellular vesicles is a critical determinant of their phenotype and function. Sci Rep 10, 18061, doi: 10.1038/s41598-020-73005-3 (2020).
33 Boing, A. N. et al. Single-Step Isolation of Extracellular Vesicles by Size-Exclusion Chromatography. J Extracell Vesicles 3, doi: 10.3402/jev.v3.23430 (2014).
34 Blans, K. et al. Pellet-Free Isolation of Human and Bovine Milk Extracellular Vesicles by Size-Exclusion Chromatography. J Extracell Vesicles 6, 1294340, doi: 10.1080/20013078.2017.1294340 (2017).
35 Clotilde Thery, A. C., Sebastian Amigorena, and Graca Raposo. Isolation and Characterization of Exosomes from Cell Culture Supernatants and Biological Fluids. Protocols in Cell Biology 3.22 (2006).
36 Helwa, I. et al. A Comparative Study of Serum Exosome Isolation Using Differential Ultracentrifugation and Three Commercial Reagents. PLoS One 12, e0170628, doi: 10.1371/journal.pone.0170628 (2017).
37 Watson, D. C. et al. Scalable, cGMP-Compatible Purification of Extracellular Vesicles Carrying Bioactive Human Heterodimeric IL-15/Lactadherin Complexes. J Extracell Vesicles 7, 1442088, doi: 10.1080/20013078.2018.1442088 (2018).
38 Cheruvanky, A. et al. Rapid Isolation of Urinary Exosomal Biomarkers Using a Nanomembrane Ultrafiltration Concentrator. Am J Physiol Renal Physiol 292, F1657-1661, doi: 10.1152/ajprenal.00434.2006 (2007).
39 Wan, Y. et al. Rapid magnetic isolation of extracellular vesicles via lipid-based nanoprobes. Nat Biomed Eng 1, doi: 10.1038/s41551-017-0058 (2017).
40 Balaj, L. et al. Heparin affinity purification of extracellular vesicles. Sci Rep 5, 10266, doi: 10.1038/srep10266 (2015).
41 Petersen, K. E. et al. A Review of Exosome Separation Techniques and Characterization of B16-F10 Mouse Melanoma Exosomes with AF4-UV-MALS-DLS-TEM. Anal Bioanal Chem 406, 7855-7866, doi: 10.1007/s00216-014-8040-0 (2014).
42 Karimi, N. et al. Detailed Analysis of the Plasma Extracellular Vesicle Proteome After Separation from Lipoproteins. Cell Mol Life Sci 75, 2873-2886, doi: 10.1007/s00018-018-2773-4 (2018).
43 Monguio-Tortajada, M., Galvez-Monton, C., Bayes-Genis, A., Roura, S. & Borras, F. E. Extracellular Vesicle Isolation Methods: Rising Impact of Size-Exclusion Chromatography. Cell Mol Life Sci 76, 2369-2382, doi: 10.1007/s00018-019-03071-y (2019).
44 Lars Hagel, M. O., Torvald Andersson. Apparent pore size distributions of chromatography media. Journal of Chromatography A 743, 33-42 (1996).
45 Reynolds, J. A. N. Y. T., Charles. Gel-Exclusion Chromatography on S1000 Sephacryl: Application to Phospholipid Vesicles. Analytical Biochemistry 130, 471-474 (1983).
46 Zawada, Z. H. A Single-Step Method Of Liposome Preparation. Cellular & Molecular Biology Letters 9, 603-615 (2004).
47 Shigeo Fujita, A. E., Jun Okabe, Atsushi Harada, Katsunori Sasaki, Naoko Ogiwara, Yoshifumi Inoue, Toshinori Ito, Hikaru Matsuda, Kazunori Kataoka, Atsushi Kato, Mamoru Hasegawa, and Mahito Nakanishi. Sendai Virus-Mediated Gene Delivery into Hepatocytes via Isolated Hepatic Perfusion. Biol. Pharm, Bull. 29, 1728-1734 (2006).
48 Nice, J. B. et al. Heterogeneity of Size and Toxin Distribution in Aggregatibacter actinomycetemcomitans Outer Membrane Vesicles. Toxins 16, doi: 10.3390/toxins16030138 (2024).
49 Brouwers, J. F. et al. Distinct lipid compositions of two types of human prostasomes. Proteomics 13, 1660-1666, doi: 10.1002/pmic.201200348 (2013).
50 Liangsupree, T., Multia, E. & Riekkola, M. L. Modern isolation and separation techniques for extracellular vesicles. J Chromatogr A 1636, 461773, doi: 10.1016/j.chroma.2020.461773 (2021).
51 Michael P. Washburn, D. W., and John R. Yates III. Large-scale analysis of the yeast proteome by multidimensional protein identification technology. Nature Biotechnology 19, 242-247 (2001).
52 Vanhoenacker, G., Vandenheede, I., David, F., Sandra, P. & Sandra, K. Comprehensive two-dimensional liquid chromatography of therapeutic monoclonal antibody digests. Anal Bioanal Chem 407, 355-366, doi: 10.1007/s00216-014-8299-1 (2015).
53 Francois, I., Sandra, K. & Sandra, P. Comprehensive liquid chromatography: fundamental aspects and practical considerations—a review. Anal Chim Acta 641, 14-31, doi: 10.1016/j.aca.2009.03.041 (2009).
54 Stoll, D. R. & Carr, P. W. Two-Dimensional Liquid Chromatography: A State of the Art Tutorial. Anal Chem 89, 519-531, doi: 10.1021/acs.analchem.6b03506 (2017).
55 Baranyai, T. et al. Isolation of Exosomes from Blood Plasma: Qualitative and Quantitative Comparison of Ultracentrifugation and Size Exclusion Chromatography Methods. PLoS One 10, e0145686, doi: 10.1371/journal.pone.0145686 (2015).
56 Zimmerman, A. J. et al. Multimode chromatography-based techniques for high purity isolation of extracellular vesicles from human blood plasma. Journal of Extracellular Biology 3, e147, doi: 10.1002/jex2.147 (2024).
57 Rembert Pieper, Q. S. & Christine L. Gatlin, S.-T. H., N. Leigh Anderson, Sandra Steiner. Multi-Component Immunoaffinity Subtraction Chromatography: An Innovative Step Towards a Comprehensive Survey of the Human Plasma Proteome. Proteomics 3 (2003).
58 Sari Lusa, M. J., Jari Metso, Pentti Somerharju, And Christian Ehnholm. The mechanism of human plasma phospholipid transfer protein-induced enlargement of high-density lipoprotein particles: evidence for particle fusion. Biochem. J. 313, 275-282 (1996).
59 Toth, E. A. et al. Formation of a protein corona on the surface of extracellular vesicles in blood plasma. J Extracell Vesicles 10, e12140, doi: 10.1002/jev2.12140 (2021).
60 Nieuwland, R. & Siljander, P. R. A beginner's guide to study extracellular vesicles in human blood plasma and serum. J Extracell Vesicles 13, e12400, doi: 10.1002/jev2.12400 (2024).
61 Baniwal, S. K., Chimge, N. O., Jordan, V. C., Tripathy, D. & Frenkel, B. Prolactin-induced protein (PIP) regulates proliferation of luminal A type breast cancer cells in an estrogen-independent manner. PLoS One 8, e62361, doi: 10.1371/journal.pone.0062361 (2014).
62 Danielson, K. M. et al. Diurnal Variations of Circulating Extracellular Vesicles Measured by Nano Flow Cytometry. PLoS One 11, e0144678, doi: 10.1371/journal.pone.0144678 (2016).
63 Kim, O., Hwangbo, C., Tran, P. T. & Lee, J. H. Syntenin-1-mediated small extracellular vesicles promotes cell growth, migration, and angiogenesis by increasing onco-miRNAs secretion in lung cancer cells. Cell Death Dis 13, 122, doi: 10.1038/s41419-022-04594-2 (2022).
64 Getz, G. S. & Reardon, C. A. Apoprotein E as a lipid transport and signaling protein in the blood, liver, and artery wall. J Lipid Res 50 Suppl, S156-161, doi: 10.1194/jlr.R800058-JLR200 (2009).
65 Morton, R. E. & Mihna, D. Apolipoprotein F concentration, activity, and the properties of LDL controlling ApoF activation in hyperlipidemic plasma. J Lipid Res 63, 100166, doi: 10.1016/j.jlr.2021.100166 (2022).
66 Buzas, E. I. Opportunities and challenges in studying the extracellular vesicle corona. Nat Cell Biol 24, 1322-1325, doi: 10.1038/s41556-022-00983-z (2022).
67 Heidarzadeh, M., Zarebkohan, A., Rahbarghazi, R. & Sokullu, E. Protein corona and exosomes: new challenges and prospects. Cell Commun Signal 21, 64, doi: 10.1186/s12964-023-01089-1 (2023).
68 Liam-Or, R. et al. Cellular uptake and in vivo distribution of mesenchymal-stem-cell-derived extracellular vesicles are protein corona dependent. Nat Nanotechnol 19, 846-855, doi: 10.1038/s41565-023-01585-y (2024).
69 Dietz, L. et al. Uptake of extracellular vesicles into immune cells is enhanced by the protein corona. J Extracell Vesicles 12, e12399, doi: 10.1002/jev2.12399 (2023).
70 Glaviano, A. et al. PI3K/AKT/mTOR signaling transduction pathway and targeted therapies in cancer. Mol Cancer 22, 138, doi: 10.1186/s12943-023-01827-6 (2023).
71 Xia, Y., Shen, S. & Verma, I. M. NF-kappaB, an active player in human cancers. Cancer Immunol Res 2, 823-830, doi: 10.1158/2326-6066.CIR-14-0112 (2014).
72 Hao, P. et al. Eukaryotic translation initiation factors as promising targets in cancer therapy. Cell Commun Signal 18, 175, doi: 10.1186/s12964-020-00607-9 (2020).
73 Villarroya-Beltri, C. et al. Sumoylated hnRNPA2B1 controls the sorting of miRNAs into exosomes through binding to specific motifs. Nat Commun 4, 2980, doi: 10.1038/ncomms3980 (2013).
74 Boudhraa, Z., Carmona, E., Provencher, D. & Mes-Masson, A. M. Ran GTPase: A Key Player in Tumor Progression and Metastasis. Front Cell Dev Biol 8, 345, doi: 10.3389/fcell.2020.00345 (2020).
75 Mackmull, M. T. et al. Landscape of nuclear transport receptor cargo specificity. Mol Syst Biol 13, 962, doi: 10.15252/msb.20177608 (2017).
76 Zuo, W. F. et al. Heat shock proteins as hallmarks of cancer: insights from molecular mechanisms to therapeutic strategies. J Hematol Oncol 17, 81, doi: 10.1186/s13045-024-01601-1 (2024).
77 Yu, H., Lin, L., Zhang, Z., Zhang, H. & Hu, H. Targeting NF-kappaB pathway for the therapy of diseases: mechanism and clinical study. Signal Transduct Target Ther 5, 209, doi: 10.1038/s41392-020-00312-6 (2020).
78 Karlenius, T. C. & Tonissen, K. F. Thioredoxin and Cancer: A Role for Thioredoxin in all States of Tumor Oxygenation. Cancers (Basel) 2, 209-232, doi: 10.3390/cancers2020209 (2010).
79 Verónica Cánovas, M. L., Juan Morote, Rosanna Paciucci. The role of prostate tumor overexpressed 1 in cancer progression. Oncotarget 8, 12451-12471 (2017).
80 Dubik, N. & Mai, S. Lamin A/C: Function in Normal and Tumor Cells. Cancers (Basel) 12, doi: 10.3390/cancers12123688 (2020).
81 Xu, L., Zhai, L., Ge, Q., Liu, Z. & Tao, R. Vacuolar Protein Sorting 4B (VPS4B) Regulates Apoptosis of Chondrocytes via p38 Mitogen-Activated Protein Kinases (MAPK) in Osteoarthritis. Inflammation 40, 1924-1932, doi: 10.1007/s10753-017-0633-2 (2017).
82 Huang, L. J., Zhan, S. T., Pan, Y. Q., Bao, W. & Yang, Y. The role of Vps4 in cancer development. Front Oncol 13, 1203359, doi: 10.3389/fonc.2023.1203359 (2023).
83 Liu, Y. et al. Vacuolar protein sorting 4B, an ATPase protein positively regulates the progression of NSCLC via promoting cell division. Mol Cell Biochem 381, 163-171, doi: 10.1007/s11010-013-1699-2 (2013).
84 Liao, Z. et al. An Internal Standard-Assisted Synthesis and Degradation Proteomic Approach Reveals the Potential Linkage between VPS4B Depletion and Activation of Fatty Acid beta-Oxidation in Breast Cancer Cells. Int J Proteomics 2013, 291415, doi: 10.1155/2013/291415 (2013).
85 Wang, Z. et al. Inhibition of FEN1 promotes DNA damage and enhances chemotherapeutic response in prostate cancer cells. Med Oncol 40, 242, doi: 10.1007/s12032-023-02110-w (2023).
86 Aseervatham, J. Interactions between 14-3-3 Proteins and Actin Cytoskeleton and Its Regulation by microRNAs and Long Non-Coding RNAs in Cancer. Endocrines 3, 665-702, doi: 10.3390/endocrines3040057 (2022).
87 Chevrollier, A., Loiseau, D., Reynier, P. & Stepien, G. Adenine nucleotide translocase 2 is a key mitochondrial protein in cancer metabolism. Biochim Biophys Acta 1807, 562-567, doi: 10.1016/j.bbabio.2010.10.008 (2011).
88 Sun, M. et al. Activation of the HNRNPA2B1/miR-93-5p/FRMD6 axis facilitates prostate cancer progression in an m6A-dependent manner. J Cancer 14, 1242-1256, doi: 10.7150/jca.83863 (2023).
89 Sato, S., Fujita, N. & Tsuruo, T. Regulation of kinase activity of 3-phosphoinositide-dependent protein kinase-1 by binding to 14-3-3. J Biol Chem 277, 39360-39367, doi: 10.1074/jbc.M205141200 (2002).
90 Yu Zhang, Z. H., Zhiqiang Zhu, Jianwei Liu, Xin Zheng, and Yuhai Zhang. Network analysis of ChIP-Seq data reveals key genes in prostate cancer. European Journal of Medical Research 19 (2014).
91 Lu, Y. et al. CRNDE mediated hnRNPA2B1 stability facilitates nuclear export and translation of KRAS in colorectal cancer. Cell Death Dis 14, 611, doi: 10.1038/s41419-023-06137-9 (2023).
92 Chandrashekar, D. S. et al. UALCAN: A Portal for Facilitating Tumor Subgroup Gene Expression and Survival Analyses. Neoplasia 19, 649-658, doi: 10.1016/j.neo.2017.05.002 (2017).
93 Chandrashekar, D. S. et al. UALCAN: An update to the integrated cancer data analysis platform. Neoplasia 25, 18-27, doi: 10.1016/j.neo.2022.01.001 (2022).
94 Liu, Y. et al. An Overview: The Diversified Role of Mitochondria in Cancer Metabolism. Int J Biol Sci 19, 897-915, doi: 10.7150/ijbs.81609 (2023).
95 Wallace, D. C. Mitochondria and cancer. Nat Rev Cancer 12, 685-698, doi: 10.1038/nrc3365 (2012).
96 Andrew J. W. Rodgers, M. C. J. W. Structure of the γ-ε complex of ATP synthase. Nature Structural Biology 7, 1051-1054 (2020).
97 Wang, T., Ma, F. & Qian, H. L. Defueling the cancer: ATP synthase as an emerging target in cancer therapy. Mol Ther Oncolytics 23, 82-95, doi: 10.1016/j.omto.2021.08.015 (2021).
98 Ding, C., Fan, X. & Wu, G. Peroxiredoxin 1—an antioxidant enzyme in cancer. J Cell Mol Med 21, 193-202, doi: 10.1111/jcmm. 12955 (2017).
99 Wang, R. et al. Identification of PRDX5 as A Target for The Treatment of Castration-Resistant Prostate Cancer. Adv Sci (Weinh) 11, e2304939, doi: 10.1002/advs.202304939 (2024).
100 Zhao, Z. & Shilatifard, A. Epigenetic modifications of histones in cancer. Genome Biol 20, 245, doi: 10.1186/s13059-019-1870-5 (2019).
101 Yang, Y., Zhang, M. & Wang, Y. The roles of histone modifications in tumorigenesis and associated inhibitors in cancer therapy. J Natl Cancer Cent 2, 277-290, doi: 10.1016/j.jncc.2022.09.002 (2022).
102 Su, Y. et al. Plasma extracellular vesicle long RNA profiles in the diagnosis and prediction of treatment response for breast cancer. NPJ Breast Cancer 7, 154, doi: 10.1038/s41523-021-00356-z (2021).
103 Cocks, A. et al. Diverse roles of EV-RNA in cancer progression. Semin Cancer Biol 75, 127-135, doi: 10.1016/j.semcancer.2020.11.022 (2021).
104 Kalluri, R. & McAndrews, K. M. The role of extracellular vesicles in cancer. Cell 186, 1610-1626, doi: 10.1016/j.cell.2023.03.010 (2023).
105 Wortzel, I. et al. Unique structural configuration of EV-DNA primes Kupffer cell-mediated antitumor immunity to prevent metastatic progression. Nat Cancer, doi: 10.1038/s43018-024-00862-6 (2024).

1. A method of isolation of extracellular vesicles (EVs), the method comprising:
(a) providing a liquid sample comprising EVs;
(b) subjecting the sample to multi-dimensional chromatography, thereby providing isolated EVs;
wherein the multi-dimensional chromatography comprises use of charge-based chromatography and at least one other chromatography method, or
wherein the multi-dimensional chromatography comprises size exclusion chromatography.

2. (canceled)

3. The method of claim 1, wherein the charge-based chromatography comprises anion exchange chromatography.

4. The method of claim 3, wherein the method comprises binding components of said liquid sample, or a sample derived therefrom, to an anion exchange chromatography medium followed by elution of EVs from the anion exchange chromatography medium using a gradient of pH or ionic strength, wherein the gradient of pH or iconic strenth is stepwise or linear.

5. (canceled)

6. The method of claim 4, wherein elution of EVs or EV sub-populations comprises stepwise elution of successive fractions using a series of buffered solutions of decreasing pH.

7. (canceled)

8. The method of claim 1, wherein the size exclusion chromatography comprises use of two or more different size exclusion chromatography media, each having a different pore size range and size fractionation range.

9. The method of claim 8, wherein an EV sub-population eluted from a first size exclusion chromatography medium is further fractionated using a second size exclusion chromatography medium having a smaller pore size range and size fractionation range than the first size exclusion chromatography medium.

10. The method of claim 9, wherein an EV sub-population eluted from the second size exclusion chromatography medium is further fractionated using at least a third size exclusion chromatography medium having a smaller pore size range and size fractionation range than the second size exclusion chromatography medium.

11. The method of claim 1, wherein the multi-dimensional chromatography comprises anion exchange chromatography and size exclusion chromatography.

12. (canceled)

13. The method of claim 1, wherein the multi-dimensional chromatography comprises performing two or more different types of chromatography using a single chromatography column or multiple chromatography columns.

14. The method of claim 1, wherein the multi-dimensional chromatography comprises subjecting the liquid sample, or a sample derived therefrom to chromatography using a chromatography medium comprising porous beads having internal charged groups, wherein said chromatography medium is capable of simultaneously performing sous size-based fractionation and anion exchange.

15. The method of claim 1, wherein the multi-dimensional chromatography comprises immunoaffinity chromatography, and wherein an EV-associated biomarker is used as affinity ligand.

16. (canceled)

17. The method of claim 1, further comprising subjecting the liquid sample to centrifugation and/or filtration, whereby cells, subcellular components, and/or lipids are removed from the liquid sample prior to performing said multi-dimensional chromatography.

18. The method of claim 1, wherein the liquid sample is, or is derived from, a liquid biopsy specimen, a cell culture medium, a cell lysate, a biological sample, a clinical sample, or an environmental sample.

19. The method of claim 1, wherein the isolated EVs are enriched in EVs compared to the liquid sample.

20. The method of claim 1, wherein the isolated EVs are fractionated into two or more fractions enriched with different EV subpopulations.

21. The method of claim 20, wherein the two or more EV subpopulations differ from one another by EV surface charge distribution or mean EV surface charge, or wherein the two or more EV subpopulations differ from one another by EV size distribution or mean EV size.

22. (canceled)

23. The method of claim 20, wherein the two or more EV subpopulations differ from one another by EV protein, lipid, glycan, or nucleic acid composition.

24. The method of claim 20, further comprising
subjecting the two or more EV subpopulations to a molecular analysis and/or morphological analysis, and wherein the molecular analysis and/or morphological analysis comprise one or more proteomic analysis, lipidomic analysis, metabolomic analysis, glycomic analysis, transcriptomic analysis, targeted mass spectrometry, direct charge-base detection mass spectrometry, analysis of post-translational and post-transcriptional modifications, immunoaffinity methods, flow cytometry, biomarker analysis, imaging by electron microscopy, fluorescence microscopy, particle size analysis, or any combination thereof.

25. (canceled)

26. (canceled)

27. The method of claim 20, wherein the two or more EV subpopulations differ from one another in protein composition, and wherein the two or more EV subpopulations differ in presence, absence, or amount of post-translational modifications, one or more oncogenic proteins, tumor suppressor proteins, tetraspanins, lipoproteins, RNA-binding proteins, histones, mitochondrial proteins, plasma proteins, or any combination thereof.

28. (canceled)

29. The method of claim 20, wherein at least one of said two or more EV subpopulations comprises one or more components associated with or diagnostic for a biological state, medical condition, or disease.

30. The method of claim 29, wherein said one or more components are diagnostic for a disease selected from the group consisting of cancers, neurodegenerative diseases, cardiovascular diseases, autoimmune diseases, infectious diseases, and aging related diseases.

31. The method of claim 30, wherein the disease is a cancer selected from the group consisting of melanoma, glioma, prostate cancer, breast cancer, cervical cancer, colorectal cancer, kidney cancer, lung cancer, lymphoma and pancreatic cancer.

32. The method of claim 1, wherein the liquid sample comprises plasma, and the isolated EVs are at least partially separated from one or more plasma proteins and/or from one or more lipoprotein particle types.

33. A method of diagnosis or prognosis of a medical condition or disease, the method comprising:
(a) providing a sample from a subject suspected of having the medical condition or disease;
(b) optionally processing the sample to provide a liquid sample suitable for use as the liquid sample of claim 1;
(c) performing the method of claim 1 using the sample of (a) or the provided liquid sample of (b) as the liquid sample of said method; and
(d) providing a diagnosis or prognosis of the medical condition or disease based on said determination of one or more components associated with or diagnostic for the medical condition or disease.

34. A method for identifying an EV-associated biomarker for a biological state, medical condition, or disease, the method comprising:
(a) performing the method of claim 24, wherein the liquid sample is obtained or derived from a subject having said biological state, medical condition, or disease, and whereby results of said proteomics analysis are obtained;
(b) comparing the results obtained in (a) with results of molecular and/or morphological analysis representing the lack of said biological state, medical condition, or disease, wherein the molecular analysis and/or morphological analysis comprise one or more proteomic analysis, analysis of post-translational modifications and proteoforms, lipidomic analysis, metabolomic analysis, glycomic analysis, transcriptomic analysis, analysis of post-transcriptional modifications, targeted mass spectrometry, direct charge-based detection mass spectrometry, immunoaffinity methods, flow cytometry, imaging by electron microscopy or fluorescence microscopy, or any combination thereof; and
(c) identifying one or more EV-associated biomarkers for said biological state, medical condition, or disease.

35. (canceled)

36. A kit comprising a chromatography device, instructions for carrying out the method of claim 1, and optionally a reagent such as an antibody for detection of an EV-associated biomarker.

37. A system for isolation, purification, and/or analysis of EVs, the system comprising a chromatography device, a liquid chromatography system capable of use with the chromatography device, and a processor and memory comprising instructions for carrying out the method of claim 1.
